(19) United States
(12) Patent Application Publication
(10) Pub. No.: US 2022/0023448 A1
(43) Pub. Date: Jan. 27, 2022

(54) DENDRIMER FOR THERAPY AND IMAGING

(71) Applicant: Starpharma Pty Ltd., Abbotsford (AU)

(72) Inventors: David James OWEN, Abbotsford (AU); Richard HUFTON, Abbotsford (AU); Kristofer James THURECHT, Abbotsford (AU)

(21) Appl. No.: 17/298,436

(22) PCT Filed: Nov. 29, 2019
(86) PCT No.: PCT/AU2019/051312

§ 371 Date: May 28, 2021

(30) Foreign Application Priority Data
Nov. 29, 2018 (AU) 2018904548

(51) Int. Cl. A61K 51/06; C08G 83/00; A61K 47/64; A61K 47/60; A61P 35/00

(52) U.S. Cl. CPC A61K 51/065; C08G 83/004; A61P 35/00; A61K 47/60; A61K 47/641

(57) ABSTRACT

Provided herein is a dendrimer comprising: i) a core unit (C); and ii) building units (BU), wherein the core unit is covalently attached to at least two building units; the dendrimer having from two to six generations of building units; wherein building units of different generations are covalently attached to one another; and the dendrimer further comprising: iii) one or more first terminal groups attached to an outermost building unit, wherein each first terminal group comprises a radionuclide-containing moiety; and iv) one or more second terminal groups attached to an outermost building unit, wherein each second terminal group comprises a pharmacokinetic-modifying moiety; or a salt thereof. Also provided are compositions comprising the dendrimers, and methods of using the dendrimers and compositions in diagnostic and therapeutic applications.

### FIELD

The present disclosure relates to dendrimers comprising a radionuclide-containing moiety. The dendrimers find use in diagnostic, theranostic and therapeutic applications, for example with imaging of tumours. The present disclosure also relates to pharmaceutical compositions comprising the dendrimers, and methods of diagnosis, imaging, determining therapy, and treatment using the dendrimers.

### BACKGROUND

Molecular imaging techniques include both single modality, such as positron emission tomography (PET), single photon emission computed tomography (SPECT), magnetic resonance imaging (MRI), magnetic resonance spectroscopy (MRS), computed tomography (CT), ultrasound, bioluminescence, fluorescence imaging and also multimodalities such as PET/CT, SPECT/CT and PET/MRI. Radionuclide-based imaging methods, especially PET, continue to be an active area of investigation for both diagnostic and therapeutic applications due to their high sensitivity (picomolar level) and limitless tissue penetration.

Radiotherapy is a powerful tool against cancer due to its ability to induce DNA damage and cell cycle arrest. Approximately 50% of cancer patients receive radiotherapy, with around 40% success. Internal radiation, predominantly delivers alpha or beta emitting radionuclides to the tumour. Existing methods of delivering radiotherapy to the desired site, while minimising deleterious off site radiation exposure includes mimetics, such as Xifigo (Ra223, Bayer) radioactive beads such as sirspheres (Y-90Sirtex), and targeted therapies such as Lutathera (AAA/Novartis). However, there is a need for therapies that allow for improved delivery of radiotherapeutics and radio imaging agents to the tumour site. In addition there is a need for radiotheranostics that allow for both imaging and therapy using the same or closely related agents.

### SUMMARY

It has been found that radiolabelling of dendrimers has great potential for enhanced sensitivity for early stage disease detection, accurate diagnosis and personalised therapy of various disease types, especially cancer. Dendrimers have the ability to present various surface functionalities on one surface, such as radionuclide complexes to provide imaging stability and pharmacokinetic modifying agents which can significantly increase solubility and provide stealth.

The invention is predicated in part on the discovery that dendrimers based on lysine or lysine analogue building units which have an outermost nitrogen atom attached to a radionuclide-containing moiety, and which have an outermost nitrogen atom attached to pharmacokinetic-modifying moiety, are unexpectedly effective in tumour imaging applications. Example radionuclide-containing dendrimers have surprisingly been found to accumulate to a high extent in tumours, including brain tumours.

Accordingly, in a first aspect, there is provided a dendrimer comprising:

i) a core unit (C); and

ii) building units (BU),

wherein the core unit is covalently attached to at least two building units;

the dendrimer having from two to six generations of building units; wherein building units of different generations are covalently attached to one another; and

the dendrimer further comprising:

iii) one or more first terminal groups attached to an outermost building unit, wherein each first terminal group comprises a radionuclide-containing moiety; and

iv) one or more second terminal groups attached to an outermost building unit, wherein each second terminal group comprises a pharmacokinetic-modifying moiety;

or a salt thereof.

In some embodiments, the first terminal group comprises a complexation group and a radionuclide. In some embodiments, the complexation group is a DOTA, benzyl-DOTA, NOTA, DTPA, sarcophagine or DFO group. In some embodiments, the complexation group is a DOTA, benzyl-DOTA, NOTA, DTPA or DFO group. In some embodiments, the radionuclide in the radionuclide-containing moiety is a lutetium, gadolinium, gallium, zirconium, actinium, bismuth, astatine, technetium or copper radionuclide. In some embodiments, the radionuclide is a gadolinium, zirconium or lutetium radionuclide. In some embodiments, the radionuclide is a copper, zirconium, lutetium, actinium or astatine radionuclide. In some embodiments, the radionuclide is a copper-64, copper-67, zirconium-89, lutetium-177, actinium-225 or an astatine-211 radionuclide. In some embodiments, the radionuclide is an α-emitter. In some embodiments, the radionuclide is a β-emitter.

In some embodiments, the pharmacokinetic-modifying moiety is a polyethylene glycol (PEG) group or a polyethyloxazoline (PEOX) group. In some embodiments, the pharmacokinetic-modifying moiety is a PEG group having an average molecular weight of at least 500 Daltons. In some embodiments, the pharmacokinetic-modifying moiety is a PEG group having an average molecular weight in the range of from 500 to 3000 Daltons. In some embodiments, the PEG group is a methoxy-terminated PEG.

In some embodiments, the dendrimer comprises a third terminal group attached to an outermost building unit, the third terminal group comprising a residue of a pharmaceutically active agent. In some embodiments, the pharmaceutically active agent is an anti-cancer agent or radiosensitiser. In some embodiments, the anticancer agent is selected from the group consisting of an auristatin, a maytansinoid, a taxane, a topoisomerase inhibitor and a nucleoside analogue. In some embodiments, the anticancer agent is selected from the group consisting of monomethyl auristatin E, monomethyl auristatin F, cabazitaxel, docetaxel, SN-38 and gemcitabine. In some embodiments, the anti-cancer agent is selected from the group consisting of cabazitaxel, docetaxel, and SN-38.

In some embodiments, the residue of a pharmaceutically active agent is covalently attached to an outermost building unit via a linker. In some embodiments, the residue of a pharmaceutically active agent is covalently attached to an outermost building unit via a cleavable linker. In some embodiments, the linker is

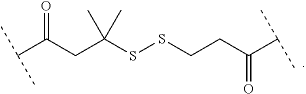

In some embodiments, the core unit does not provide an attachment point for a terminal group other than via the building units.

In some embodiments, the generations of building units are complete generations.

In some embodiments, the core unit is covalently attached to at least two building units via amide linkages, each amide linkage being formed between a nitrogen atom present in the core unit and the carbon atom of an acyl group present in a building unit. In some embodiments, the core unit of the dendrimer is formed from a core unit precursor comprising two amino groups. In some embodiments, the core unit is:

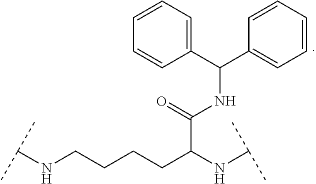

In some embodiments, building units of different generations are covalently attached to one another via amide linkages formed between a nitrogen atom present in one building unit and the carbon atom of an acyl group present in another building unit. In some embodiments, the building units are lysine residues or analogues thereof. In some embodiments, the building units are each:

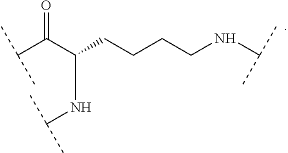

In some embodiments, the first terminal group is attached to the nitrogen atom of an outermost building unit, and the second terminal group is attached to the nitrogen atom of an outermost building unit. In some embodiments, from 1 to 3 of the nitrogen atoms present in the outermost building units are attached to a first terminal group. In some embodiments, at least 40% of the nitrogen atoms present in the outermost building units are attached to a second terminal group.

In some embodiments, the dendrimer comprises a third terminal group attached to the nitrogen atom of an outermost building unit, the third terminal group comprising a residue of a pharmaceutically active agent. In some embodiments, the pharmaceutically active agent comprises a hydroxyl group, wherein the residue of a pharmaceutically active agent is covalently attached via the oxygen atom of the hydroxyl group through a cleavable linker to an outermost building unit, and wherein the cleavable linker is a diacyl linker group. In some embodiments, the diacyl linker group is of formula

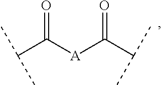

wherein A is a C₂-C₁₀ alkylene group which is optionally interrupted by O, S, S—S, NH, or N(Me), or in which A is a heterocycle selected from the group consisting of tetrahydrofuran, tetrahydrothiophene, pyrrolidine and N-methylpyrrolidine. In some embodiments, the diacyl linker is

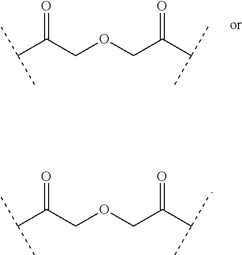

In some embodiments, at least one third of the nitrogen atoms present in the outermost building units are attached to a third terminal group.

In some embodiments, the dendrimer comprises outermost building units which contain —NH₂ groups and/or which contain a nitrogen atom which is capped with an acetyl group. In some embodiments, at least 80% of the nitrogen atoms present in the outermost generation of building units are substituted.

In some embodiments, the dendrimer comprises surface units comprising an outer building unit and a second terminal group of the formula:

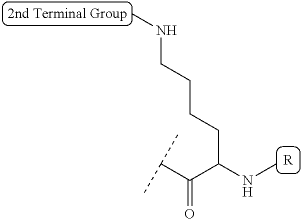

wherein R represents a first terminal group or a third terminal group.

In some embodiments, the dendrimer is any one of the Example dendrimers as described herein.

In another aspect, there is provided a composition comprising a plurality of dendrimers or salts thereof,

wherein at least some of the dendrimers in the composition are as described herein according to any one or more of the aspects, embodiments or examples thereof,

the mean number of first terminal groups per dendrimer in the composition is in the range of from about 0.2 to 8, and

the mean number of second terminal groups per dendrimer in the composition is in the range of from about 10 to 32.

In some embodiments, the mean number of third terminal group per dendrimer in the composition is in the range of from about 10 to 31. In some embodiments, the composition is a pharmaceutical composition comprising a pharmaceutically acceptable excipient.

In another aspect, there is provided a method of determining whether a subject has a cancer, comprising:

administering to a subject a dendrimer as described herein according to any one of the aspects, embodiments or examples thereof or a pharmaceutical composition as described herein according to any one or more of the aspects, embodiments or examples thereof;

carrying out imaging on the subject's body or a part thereof, and

determining whether the subject has a cancer based on the imaging results.

In another aspect, there is provided a method of imaging a cancer in a subject, comprising:

administering to a subject having a cancer a dendrimer as described herein according to any one or more of the aspects, embodiments or examples thereof or a pharmaceutical composition as described herein according to any one or more of the aspects, embodiments or examples thereof; carrying out imaging on the subject's body or a part thereof.

In another aspect, three is provided a method of determining the progression of a cancer in a subject, comprising:

administering to a subject having a cancer a first amount of a dendrimer as described herein according to any one or more of the aspects, embodiments or examples thereof or a pharmaceutical composition as described herein according to any one or more of the aspects, embodiments or examples thereof;

carrying out a first imaging step on the subject's body or a part thereof; subsequently administering to the subject a second amount of a dendrimer as described herein according to any one or more of the aspects, embodiments or examples thereof or a pharmaceutical composition as described herein according to any one or more of the aspects, embodiments or examples thereof;

carrying out a second imaging step on the subject's body or a part thereof; and

determining whether the cancer has progressed based on the first and second imaging results.

In another aspect, there is provided a method of determining an appropriate therapy for a subject having a cancer, comprising:

administering to the subject a dendrimer as described herein according to any one or more of the aspects, embodiments or examples thereof or a pharmaceutical composition as described herein according to any one or more of the aspects, embodiments or examples thereof;

carrying out imaging on the subject's body or a part thereof; and

determining if the imaging results indicate susceptibility of the cancer to treatment with a therapy, administering the therapy to the subject.

In another aspect, there is provided a method of determining the effectiveness of a cancer therapy administered to a subject having a cancer, comprising:

administering to the subject a first amount of a dendrimer as described herein according to any one or more of the aspects, embodiments or examples thereof or a pharmaceutical composition as described herein according to any one or more of the aspects, embodiments or examples thereof;

carrying out a first imaging step on the subject's body or a part thereof;

administering to the subject a cancer therapy;

subsequently administering to the subject a second amount of a dendrimer as described herein according to any one or more of the aspects, embodiments or examples thereof or a pharmaceutical composition as described herein according to any one or more of the aspects, embodiments or examples thereof;

carrying out a second imaging step on the subject's body or a part thereof; and

determining the effectiveness of the cancer therapy based on the first and second imaging results.

In some embodiments of any of the above methods where a therapy is administered, the therapy is a dendrimer as described herein according to any one or more of the aspects, embodiments or examples thereof or a pharmaceutical composition as described herein according to any one or more of the aspects, embodiments or examples thereof.

In another aspect, there is provided a method of treating cancer comprising administering to a subject in need thereof a therapeutically effective amount of a dendrimer as described herein according to any one or more of the aspects, embodiments or examples thereof or a pharmaceutical composition as described herein according to any one or more of the aspects, embodiments or examples thereof.

In another aspect, there is provided a dendrimer as described herein according to any one or more of the aspects, embodiments or examples thereof, or a pharmaceutical composition as described herein according to any one or more of the aspects, embodiments or examples thereof, for use in the diagnosis of cancer in a subject, for use in determining an appropriate therapy for a subject having a cancer, for use in determining the progression of a cancer, or for use in determining the effectiveness of a cancer therapy.

In another aspect, there is provided a dendrimer as described herein according to any one or more of the aspects, embodiments or examples thereof, or a pharmaceutical composition as described herein according to any one or more of the aspects, embodiments or examples thereof, for use in the treatment of cancer.

In another aspect, there is provided use of a dendrimer as described herein according to any one or more of the aspects, embodiments or examples thereof, or use of a pharmaceutical composition as described herein according to any one or more of the aspects, embodiments or examples thereof, in the manufacture of a medicament for the diagnosis of cancer, or for determining an appropriate therapy for a subject having a cancer, or for determining the progression of a cancer, or for determining the effectiveness of a cancer therapy.

In another aspect, there is provided use of a dendrimer as described herein according to any one or more of the aspects, embodiments or examples thereof, or of a pharmaceutical composition as described herein according to any one or more of the aspects, embodiments or examples thereof, in the manufacture of a medicament for the treatment of cancer.

In some embodiments, the cancer is prostate cancer, pancreatic cancer, gastrointestinal cancer, stomach cancer, lung cancer, uterine cancer, breast cancer, brain cancer or ovarian cancer. In some embodiments, the cancer is prostate cancer, pancreatic cancer, breast cancer or brain cancer. In some embodiments, the cancer is a brain cancer of a glioblastoma, meningioma, pituitary, nerve sheath, astrocytoma, oligodendroglioma, ependymoma, medulloblastoma, or craniopharyngioma.

In some embodiments, the dendrimer is administered in combination with a further anti-cancer drug.

In another aspect, there is provided an intermediate for producing a radionuclide-containing dendrimer which comprises:

i) a core unit (C); and

ii) building units (BU);

wherein the core unit is covalently attached to at least two building units;

the dendrimer having from two to six generations of building units; wherein building units of different generations are covalently attached to one another; and

the dendrimer further comprising:

iii) one or more first terminal groups attached to an outermost building unit, wherein each first terminal group comprises a complexation group for complexing a radionuclide; and

iv) one or more second terminal groups attached to an outermost building unit, wherein each second terminal group comprises a pharmacokinetic-modifying moiety;

or a salt thereof.

In another aspect, there is provided a kit for producing a dendrimer as described herein according to any one or more of the aspects, embodiments or examples thereof, comprising:

a) an intermediate for producing a radionuclide-containing dendrimer as described herein according to any one or more of the embodiments or examples thereof; and

b) a radionuclide.

In another aspect, there is provided a process for producing a dendrimer as described herein according to any one or more of the aspects, embodiments or examples thereof, comprising:

contacting an intermediate as defined herein with a radionuclide, thereby producing the radionuclide-containing dendrimer.

It will be appreciated that further aspects, embodiments, and examples, are described herein, which may include any one or more of the aspects, embodiments or examples as described above.

### BRIEF DESCRIPTION OF THE DRAWINGS

FIG. 1 shows a radio-TLC image for dendrimer compounds 1b and 3 labelled with ⁸⁹Zr.

FIG. 2 shows representative in vivo images showing biodistribution of dendrimer compound 1b labelled with ⁸⁹Zr in mice (n=4) bearing DU-145 xenograft at 9 days (216 hours) post-injection. The tumour is marked with a white arrow in the images.

FIG. 3 shows representative in vivo images showing biodistribution of dendrimer compound 1b labelled with ⁸⁹Zr in mice (n=4) bearing PC3 xenograft at 9 days (216 hours) post-injection. The tumour is marked with a white arrow in the images.

FIG. 4 shows representative in vivo images showing biodistribution of dendrimer compound 3 labelled with ⁸⁹Zr in mice (n=4) bearing DU-145 xenograft at 9 days (216 hours) post-injection. The tumour is marked with a white arrow in the images.

FIG. 5 shows representative in vivo images showing biodistribution of dendrimer compound 3 labelled with ⁸⁹Zr in mice (n=4) bearing PC3 xenograft at 9 days (216 hours) post-injection. The tumour is marked with a white arrow in the images.

FIG. 6 shows a chart showing in vivo biodistribution for cohorts of mice administered dendrimer compound 1b or 3 labelled with ⁸⁹Zr, in DU145 and PC3 prostate cancer xenografts, at 8 hours post-injection.

FIG. 7 shows a chart showing in vivo biodistribution for cohorts of mice administered dendrimer compound 1b or 3 labelled with ⁸⁹Zr, in DU145 and PC3 prostate cancer xenografts, at 9 days post-injection.

FIG. 8 shows a chart showing a plot of relative accumulation as a function of time, for cohorts of mice administered dendrimer compound 1b or 3 labelled with ⁸⁹Zr, in DU145 and PC3 prostate cancer xenografts.

FIG. 9 shows a radio-TLC image for dendrimer compounds 1b and 3 labelled with ⁸⁹Zr.

FIG. 10 shows representative in vivo images showing biodistribution of dendrimer compound 1b labelled with ⁸⁹Zr in mice (n=4) bearing MDA-MB-468 xenograft at 9 days (216 hours) post-injection. The tumour is marked with a white arrow in the images.

FIG. 11 shows representative in vivo images showing biodistribution of dendrimer compound 3 labelled with ⁸⁹Zr in mice (n=4) bearing MDA-MB-468 xenograft at 9 days (216 hours) post-injection. The tumour is marked with a white arrow in the images.

FIG. 12 shows representative in vivo images showing biodistribution of dendrimer compound 1b labelled with ⁸⁹Zr in mice (n=4) bearing PANC-1 xenograft at 9 days (216 hours) post-injection. The tumour is marked with a white arrow in the images.

FIG. 13 shows representative in vivo images showing biodistribution of dendrimer compound 3 labelled with ⁸⁹Zr in mice (n=4) bearing PANC-1 xenograft at 9 days (216 hours) post-injection. The tumour is marked with a white arrow in the images.

FIG. 14 shows a chart showing in vivo biodistribution for cohorts of mice administered dendrimer compound 1b or 3 labelled with ⁸⁹Zr, in MDA-MB-468 and PANC-1 breast and pancreatic cancer xenografts, at 8 hours post-injection.

FIG. 15 shows a chart showing in vivo biodistribution for cohorts of mice administered dendrimer compound 1b or 3 labelled with ⁸⁹Zr, in MDA-MB-468 and PANC-1 breast and pancreatic cancer xenografts, at 9 days post-injection.

FIG. 16 shows a chart showing a plot of relative accumulation as a function of time, for cohorts of mice administered dendrimer compound 1b or 3 labelled with ⁸⁹Zr, in MDA-MB-468 and PANC-1 breast and pancreatic cancer xenografts.

FIG. 17 shows PET-MR images of glioma-bearing mouse 40 hours post-injection of dendrimer compound 1b labelled with ⁸⁹Zr. The region of the tumour is shown with white arrows.

FIG. 18 shows PET-MR images of glioma-bearing mouse 5 days post-injection of dendrimer compound 1b labelled with ⁸⁹Zr. The region of the tumour is shown with white arrows.

FIG. 19 shows a chart showing ex vivo biodistribution for cohorts of mice administered dendrimer compound 1b or 3 labelled with ⁸⁹Zr, in DU145, PC3, MDA-MB-468 and PANC-1 breast and pancreatic cancer xenografts, at 9 days post-injection.

FIG. 20 shows a chart showing percentage change in tumour volume over time for cohorts of mice administered dendrimer compound 4b, 5 and/or a Cabazitaxel containing dendrimer.

### DESCRIPTION

### General Definitions

Unless specifically defined otherwise, all technical and scientific terms used herein shall be taken to have the same meaning as commonly understood by one of ordinary skill in the art (e.g., chemistry, biochemistry, medicinal chemistry, polymer chemistry, and the like).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−20%, more preferably +/−10%, of the designated value.

As used herein, the terms “a”, “an” and “the” include both singular and plural aspects, unless the context clearly indicates otherwise.

Unless otherwise indicated, terms such as “first,” “second,” etc. are used herein merely as labels, and are not intended to impose ordinal, positional, or hierarchical requirements on the items to which these terms refer. Moreover, reference to a “second” item does not require or preclude the existence of lower-numbered item (e.g., a “first” item) and/or a higher-numbered item (e.g., a “third” item).

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example and without limitation, two of item A, one of item B, and ten of item C; four of item B and seven of item C; or some other suitable combination.

As used herein, the term “subject” refers to any organism that is susceptible to a disease or condition. For example, the subject can be an animal, a mammal, a primate, a livestock animal (e.g., sheep, cow, horse, pig), a companion animal (e.g., dog, cat), or a laboratory animal (e.g., mouse, rabbit, rat, guinea pig, hamster). In one example, the subject is a mammal. In one embodiment, the subject is human. In one embodiment, the subject is a non-human animal.

As used herein, the term “treating” includes alleviation of symptoms associated with a specific disorder or condition. For example, as used herein, the term “treating cancer” includes alleviating symptoms associated with cancer. In one embodiment, the term “treating cancer” refers to a reduction in cancerous tumour size. In one embodiment, the term “treating cancer” refers to an increase in progression-free survival. As used herein, the term “progression-free survival” refers to the length of time during and after the treatment of cancer that a patient lives with the disease, i.e., cancer, but does not have a recurrence or increase in symptoms of the disease.

As used herein, the term “prevention” includes prophylaxis of the specific disorder or condition. For example, as used herein, the term “preventing cancer” refers to preventing the onset or duration of the symptoms associated with cancer. In one embodiment, the term “preventing cancer” refers to slowing or halting the progression of the cancer. In one embodiment, the term “preventing cancer” refers to slowing or preventing metastasis.

The term “therapeutically effective amount”, as used herein, refers to a dendrimer being administered in an amount sufficient to alleviate or prevent to some extent one or more of the symptoms of the disorder or condition being treated. The result can be the reduction and/or alleviation of the signs, symptoms, or causes of a disease or condition, or any other desired alteration of a biological system. In one embodiment, the term “therapeutically effective amount” refers to a dendrimer being administered in an amount sufficient to result in a reduction in cancerous tumour size. In one embodiment, the term “therapeutically effective amount” refers to a dendrimer being administered in an amount sufficient to result in an increase in progression-free survival. The term, an “effective amount”, as used herein, refers to an amount of a dendrimer effective to achieve a desired pharmacologic effect or therapeutic improvement without undue adverse side effects or to achieve a desired pharmacologic effect or therapeutic improvement with a reduced side effect profile. Therapeutically effective amounts may for example be determined by routine experimentation, including but not limited to a dose escalation clinical trial. The term “therapeutically effective amount” includes, for example, a prophylactically effective amount. In one embodiment, a prophylactically effective amount is an amount sufficient to prevent metastasis. It is understood that “an effective amount” or “a therapeutically effective amount” can vary from subject to subject, due to variation in metabolism of the compound and any of age, weight, general condition of the subject, the condition being treated, the severity of the condition being treated, and the judgment of the prescribing physician. An appropriate “effective amount” in any individual case may be determined by one of ordinary skill in the art using routine experimentation.

As used herein, the term “alkyl” refers to a monovalent straight-chain (i.e. linear) or branched saturated hydrocarbon group. In one example, an alkyl group contains from 1 to 10 carbon atoms ((i.e. C₁₋₁₀alkyl). In one example, an alkyl group contains from 1 to 6 carbon atoms (i.e. C₁₋₆ alkyl). Examples of alkyl groups include methyl, ethyl, propyl (e.g. n-propyl, iso-propyl), butyl (e.g. n-butyl, sec-butyl, tert-butyl), pentyl and hexyl groups.

As used herein, the term “alkylene” refers to a divalent straight-chain (i.e. linear) or branched saturated hydrocarbon group. In one example, an alkylene group contains from 2 to 10 carbon atoms ((i.e. C₂₋₁₀ alkylene). In one example, an alkylene group contains from 2 to 6 carbon atoms (i.e. C₂₋₆ alkylene). Examples of alkylene groups include, for example, —CH₂CH₂—, —CH₂CH₂CH₂—, —CH₂CH(CH₃)—, —CH₂CH₂CH₂CH₂—, —CH₂CH(CH₃)CH₂—, and the like.

Suitable salts of the dendrimers include those formed with organic or inorganic acids or bases. As used herein, the phrase “pharmaceutically acceptable salt” refers to pharmaceutically acceptable organic or inorganic salts. Exemplary acid addition salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. Exemplary base addition salts include, but are not limited to, ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion. It will also be appreciated that non-pharmaceutically acceptable salts also fall within the scope of the present disclosure since these may be useful as intermediates in the preparation of pharmaceutically acceptable salts or may be useful during storage or transport.

Those skilled in the art of organic and/or medicinal chemistry will appreciate that many organic compounds can form complexes with solvents in which they are reacted or from which they are precipitated or crystallized. These complexes are known as “solvates”. For example, a complex with water is known as a “hydrate”. As used herein, the phrase “pharmaceutically acceptable solvate” or “solvate” refer to an association of one or more solvent molecules and a compound of the present disclosure. Examples of solvents that form pharmaceutically acceptable solvates include, but are not limited to, water, isopropanol, ethanol, methanol, DMSO, ethyl acetate, acetic acid, and ethanolamine.

As used herein, the term “dendrimer” refers to a molecule containing a core and dendrons attached to the core. Each dendron is made up of generations of branched building units resulting in a branched structure with increasing number of branches with each generation of building units. A dendrimer may include pharmaceutically acceptable salts or solvates as defined supra.

As used herein, the term “building unit” refers to a branched molecule comprising functional groups, at least one functional group for attachment to the core or a previous generation of building units and at least two functional groups for attachment to the next generation of building units or forming the surface of the dendrimer molecule.

As used herein, the term “attached” refers to a connection between chemical components by way of covalent bonding. The term “covalent bonding” is used interchangeably with the term “covalent attachment”.

### Dendrimers

In a first aspect there is provided a dendrimer comprising:

i) a core unit (C); and

ii) building units (BU),

wherein the core unit is covalently attached to at least two building units;

the dendrimer having from two to six generations of building units; wherein building units of different generations are covalently attached to one another; and

the dendrimer further comprising:

iii) one or more first terminal groups attached to an outermost building unit, wherein each first terminal group comprises a radionuclide-containing moiety; and

iv) one or more second terminal groups attached to an outermost building unit, wherein each second terminal group comprises a pharmacokinetic-modifying moiety;

or a salt thereof.

The dendrimers of the present disclosure, containing a dendrimeric scaffold incorporating pharmacokinetic modifying groups and radionuclide-containing moieties, have been found to be excellent imaging agents which accumulate in tumours and provide excellent imaging properties, such as with PET imaging. Moreover, the dendrimers are effective at accumulating in brain tumours such as glioblastoma and have been observed to cross the blood-brain barrier, which further supports that they have useful imaging, diagnostic and therapeutic properties.

### Core Unit

The core unit (C) of the dendrimer provides an attachment point for dendrons formed of building units. Any suitable core unit which contains functional groups that can form covalent linkages with functional groups present on building units may be utilised.

In some embodiments, the core unit is covalently attached to at least two building units via amide linkages. In some embodiments, each amide linkage is formed between a nitrogen atom present in the core unit and the carbon atom of an acyl group present in a building unit. In other embodiments, each amide linkage is formed between the carbon atom of an acyl group present in the core unit and a nitrogen atom present in a building unit.

In some embodiments, the core unit is covalently attached to 2, 3 or 4 building units. In one particular embodiment, the core unit is covalently attached to 2 building units. The core unit may for example be formed from a core unit precursor comprising amino groups. As another example, the core unit may be formed from a core unit precursor comprising carboxylic acid groups. In the case of a core unit which is attached to 2 building units, the core unit of the dendrimer may for example be formed from a core unit precursor comprising two amino groups. In some embodiments, the core unit is:

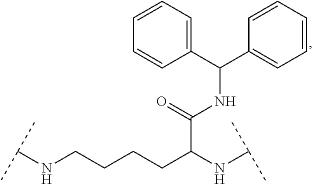

i.e. whereby the core unit comprises a lysine residue in which the acid moiety has been capped with a benzyhydrylamine (BHA-Lys) to form the corresponding amide, and may, for example, be formed from a core unit precursor:

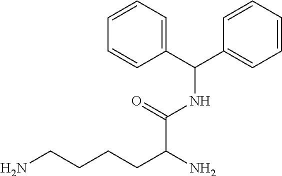 [SMILES: NCCCCC(N)C(=O)NC(c1ccccc1)c1ccccc1]

having two reactive (amino) nitrogens.

The present dendrimers allow for multiple terminal groups, to be presented on the surface of the dendrimers in a controlled manner. In particular, for lysine building units, the placement on alpha or epsilon nitrogen atoms of the building units can be predetermined as described below. In some preferred embodiments, all of the complexation groups (radionuclide-containing moieties, and complexation groups containing stable isotopes (cold material)), pharmacokinetic modifying groups and, where present, residues of pharmaceutically active agents) are provided on the surface of the dendrimer via attachment through the building units. In other words, in those embodiments, the core unit does not provide an attachment point for a terminal group other than via the building units. It will be understood that, in such embodiments, any functional groups present in the core unit which are not used for covalent attachment to a building unit will either be unreacted, or will have been capped with a suitable capping group to prevent further reaction. An example of such a core unit is the BHA-Lys group discussed above.

### Building Units

Any suitable building unit (BU) may be used to produce the dendrimers, as long as it contains a first functional group which is capable of forming a linkage with a functional group present on another building unit or a core unit, and contains at least two further functional groups which (e.g. following deprotection) are capable of forming a linkage with a functional group present on another building unit. In some preferred embodiments, building units of different generations are covalently attached to one another via amide linkages formed between a nitrogen atom present in one building unit and the carbon atom of an acyl group present in another building unit. For example, in some embodiments, the building units are lysine residues or analogues thereof, and may be formed from suitable building unit precursors, e.g. lysine or lysine analogues containing appropriate protecting groups. Lysine analogues have two amino nitrogen atoms for bonding to a subsequent generation of building units and an acyl group for bonding to a previous generation of building units or a core. Examples of suitable building units include:

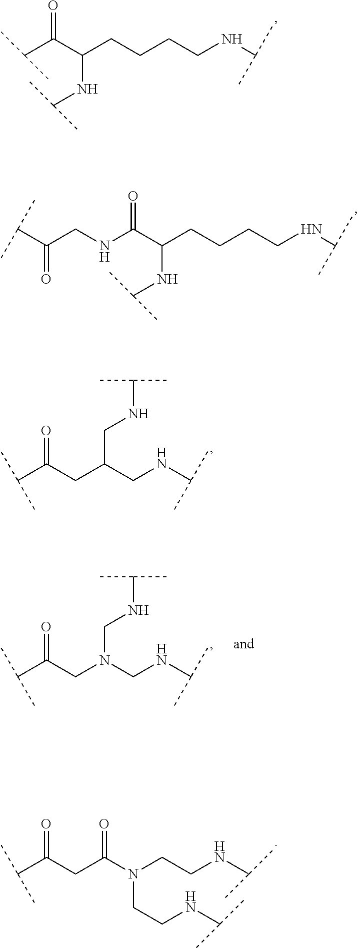

wherein the acyl group of each building unit provides a covalent attachment point for attachment to the core or to a previous generation building unit; and wherein each nitrogen atom provides a covalent attachment point which may be used for covalent attachment to a subsequent generation building unit, or to a terminal group.

In some preferred embodiments, the building units are each:

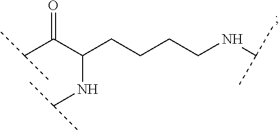

wherein the acyl group of each building unit provides a covalent attachment point for attachment to the core or to a previous generation building unit; and wherein each nitrogen atom provides a covalent attachment point which may be used for covalent attachment to a subsequent generation building unit, or to a terminal group.

In some preferred embodiments, the building units are each:

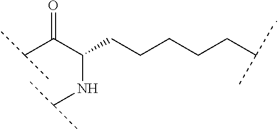

In other embodiments, the building units are aspartic acid residues, glutamic acid residues or analogues thereof, i.e. formed from suitable precursors e.g. aspartic acid, glutamic acid or analogues thereof, containing suitable protecting groups. In such embodiments, the core unit may be formed from a core unit precursor comprising carboxylic acid groups (i.e. which can react with amino groups present in the aspartic acid/glutamic acid/analogues.

The outermost generation of building units (BUₒᵤₜₑᵣ) may be formed by building units as used in the other generations of building units (BU) as described above, for example lysine or lysine analogue building units. The outermost generation of building units (BUₒᵤₜₑᵣ) is the generation of building units that is outermost from the core of the dendrimer, i.e., no further generations of building units are attached to the outermost generation of building units (BUₒᵤₜₑᵣ).

It will be appreciated that the dendrons of the dendrimer may for example be synthesised to the required number of generations through the attachment of building units (BU) accordingly. In some embodiments each generation of building units (BU) may be formed of the same building unit, for example all of the generations of building units may be lysine building units. In some other embodiments, one or more generations of building units may be formed of different building units to other generations of building units.

The dendrimer has from two to six generations of building units, i.e. 2, 3, 4, 5 or 6 generations of building units.

In some embodiments, the dendrimer has three generations of building units. A three generation building unit dendrimer is a dendrimer having a structure which includes three building units that are covalently linked to each other, for example in the case where the building units are lysines, it may comprise the substructure:

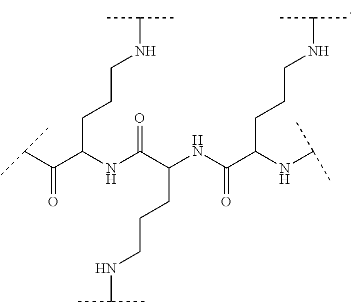

In some embodiments, the dendrimer has five generations of building units. A five generation building unit dendrimer is a dendrimer having a structure which includes five building units which are covalently linked to each another, for example in the case where the building units are lysines, it may comprise the substructure:

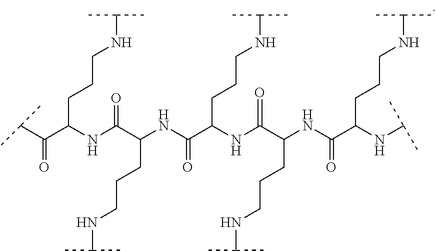

In some embodiments, the generations of building units are complete generations. For example, where the dendrimer has three generations of building units, in some embodiments the dendrimer has three complete generations of building units. With a core having two reactive amine groups, such a dendrimer will comprise 14 building units (i.e. core unit+2 BU+4 BU+8 BU).

Similarly, for example, where the dendrimer has five generations of building units, in some embodiments the dendrimer has five complete generations of building units. With a core having two reactive amine groups, such a dendrimer will comprise 62 building units (i.e. core unit+2 BU+4 BU+8 BU+16 BU+32 BU).

However, it will be appreciated that, due to the nature of the synthetic process for producing the dendrimers, one or more reactions carried out to produce the dendrimers may not go fully to completion. Accordingly, in some embodiments, the dendrimer may comprise incomplete generations of building units. For example, a population of dendrimers may be obtained, in which the dendrimers have a distribution of numbers of building units per dendrimer.

In some embodiments, where the dendrimer has three generations of building units, a population of dendrimers is obtained which has a mean number of building units per dendrimer of at least 8, or at least 9, or at least 10, or at least 11, or at least 12, or at least 13. In some embodiments, a population of dendrimers is obtained in which at least 60%, at least 70%, at least 80%, at least 90% or at least 95% of the dendrimers have 10 or more building units. In some embodiments, a population of dendrimers is obtained in which at least 60%, at least 70%, at least 80%, at least 90% or at least 95% of the dendrimers have 12 or more building units.

In some embodiments, where the dendrimer has five generations of building units, a population of dendrimers is obtained which has a mean number of building units per dendrimer of at least 55, or at least 56, or at least 57, or at least 58, or at least 59, or at least 60. In some embodiments, a population of dendrimers is obtained in which at least 60%, at least 70%, at least 80%, at least 90% or at least 95% of the dendrimers have 55 or more building units. In some embodiments, a population of dendrimers is obtained in which at least 60%, at least 70%, at least 80%, at least 90% or at least 95% of the dendrimers have 60 or more building units.

In some embodiments, each reactive (amino) group of the core unit precursor represents a conjugation site for a dendron comprising building units.

In some embodiments, each generation of building units in each dendron (X) may be represented by the formula [BU]₂₁^{(b-1)}, wherein b is the generation number. A dendron (X) having three complete generations of building units is represented as

[BU]₁-[BU]₂-[BU]₄.

A dendron (X) having five complete generations of building units is represented as

[BU]₁-[BU]₂-[BU]₄-[BU]₈-[BU]₁₆.

### First Terminal Group

The first terminal group (T1) comprises a radionuclide-containing moiety. Typically, the radionuclide-containing moiety comprises a radionuclide and a complexation group.

### Radionuclide

Any suitable radionuclide may be utilised in the present dendrimers. A radionuclide, also known as a radioactive isotope, is an un unstable form of a chemical element that radioactively decays, resulting in the emission of nuclear radiation.

Radionuclides are used in the fields of medical diagnosis and therapy. Techniques such as single photon emission, positron emission tomography (PET) imaging, and positron emission tomography—magnetic resonance imaging (PET-MRI) can be used to detect a radionuclide within a subject administered a suitable radionuclide-containing substance, and produce images which inform as to the existence and/or progression of diseases such as tumours. Radionuclides also have application in treatment of diseases, such as cancers. In such cases, administration of a radionuclide-containing substance to a patient results in delivery of radionuclide to the tumour and, following radioactive decay and emission of radiation, killing of tumour cells.

Preferably, the radionuclide is a metal radionuclide, e.g. a metal ion. In some embodiments the radionuclide is an alpha emitter (α-emitter). In some embodiments the radionuclide is an beta emitter (β-emitter). In some embodiments the radionuclide is an beta and gamma emitter.

In some embodiments the radionuclide is an actinium (e.g. Ac²²¹), astatine (e.g. As²¹¹), bismuth (e.g. Bi²¹², Bi²¹³), lead (e.g. Pb²¹²), technetium (e.g. Tc⁹⁹m), thorium (e.g. Th²²⁷), radium (e.g. Ra²²³), lutetium (e.g. Lu¹⁷⁷), yttrium (e.g. Y⁹⁰), indium (e.g. In¹¹¹, In¹¹⁴), gadolinium (e.g. Gd¹⁵³) gallium (e.g. Ga⁶⁸), zirconium (e.g. Zr⁸⁹), or copper radionuclide. In some embodiments, the radionuclide is a lutetium (e.g. Lu¹⁷), gadolinium, gallium (e.g. Ga⁶⁸), zirconium (e.g. Zr⁸⁹), actinium (e.g. Ac²²⁵), bismuth (e.g. Bi²¹², Bi²¹³), astatine (e.g. As²¹¹), technetium (e.g. Tc^{99m}), or copper (e.g. Cu⁶⁰, Cu⁶¹, Cu⁶², Cu⁶⁴, Cu⁶⁷) radionuclide. In some embodiments, the radionuclide is a lutetium (e.g. Lu¹⁷), gadolinium, gallium (e.g. Ga⁶⁸), zirconium (e.g. Zr⁸⁹), or copper (e.g. Cu⁶⁰, Cu⁶¹, Cu⁶², Cu⁶⁴, Cu⁶⁷) radionuclide. In some embodiments, the radionuclide is a gallium (e.g. Ga⁶⁸), zirconium (e.g. Zr⁸⁹) or lutetium (e.g. Lu¹⁷⁷) radionuclide. In some embodiments, the radionuclide is a copper (e.g. Cu⁶⁴, Cu⁶⁷), zirconium (e.g. Zr⁸⁹), lutetium (e.g. Lu¹⁷), actinium (e.g. Ac²²⁵) or astatine (e.g. As²¹¹) radionuclide.

In some embodiments, the radionuclide is for diagnosis or imaging of a condition (e.g. a cancer). Examples of such radionuclides include gallium (e.g. Ga⁶⁸), technetium (e.g. Tc^{99m}) zirconium (e.g. Zr⁸⁹) and, copper (e.g. Cu⁶⁰, Cu⁶¹, Cu⁶², Cu⁶⁴)

In some embodiments, the radionuclide is for treatment of a condition (e.g. a cancer). Examples of such radionuclides include actinium (e.g. Ac²²⁵), astatine (e.g. As²¹¹), bismuth (e.g. Bi²¹², Bi²¹³), lead (e.g. Pb²¹²), thorium (e.g. Th²²⁷), radium (e.g. Ra²²³1), lutetium (e.g. Lu¹⁷⁷), yttrium (e.g. Y⁹⁰), gadolinium (e.g. Gd¹⁵³), and copper (e.g. Cu⁶⁰, Cu⁶¹, Cu⁶², Cu⁶⁴)

Ideally, the emission characteristics of a therapeutic radionuclide should take into consideration the lesion size to focus energy within the tumour, and have a suitable half life to align with the extended delivery of the dendrimer. In some embodiments the radionuclide is an alpha emitter with a half life of less than 20 days or less than 12 days. In some embodiments the radionuclide is a beta emitter with a half life of 2 to 20 days or 5 to 10 days. 177Lu is a medium-energy β-emitter (490 keV) with a maximum energy of 0.5 MeV and a maximal tissue penetration of <2 mm. 177Lu also emits low-energy 7-rays at 208 and 113 keV, which allows for ex vivo imaging and consequently the collection of information pertaining to tumour localisation and dosimetry.

As would be understood by the person skilled in the art, radioactivity is measured in becquerel (Bq). One becquerel is defined as the activity of a quantity of radioactive material in which one nucleus decays per second.

In some embodiments injected doses of therapeutic radionuclide are from 1 to 50 GBq per single injection. In other embodiments injected doses are from 2 to 20 GBq per single injection/infusion. In other embodiments injected doses are from 2 to 10 GBq per single injection. Dose calculations for individual patients may be determined from a combination of disease burden, patient weight and renal function. Image-based dosimetry at each cycle of treatment is recommended, e.g. with SPECT-CT.

In some embodiments, the dendrimer is provided in a composition as a unit dosage form, e.g. having a desired level of radioactivity.

In some embodiments, the radionuclide is formulated in a unit dosage composition, such that each unit dosage contains an amount of radionuclide which has a radioactivity in the range of from 0.1 to 10 MBq, from 0.1 to 5 MBq, from 0.1 to 2 MBq, from 0.1 to 1 MBq, from 0.5 to 10 MBq, from 1 to 10 MBq, from 1 to 5 MBq, from 5 to 10 MBq, or about 1, about 2, about 3, about 4, about 5, about 6, about 7, about 8, about 9 or about 10 MBq.

For example, where the unit dosage is in the form of an injection/infusion, the injection/infusion will be formulated such that the desired amount of radiation is delivered to the target site (e.g., tumour). In some embodiments, the radionuclide is provided in a unit dosage composition for injection, such that each unit dosage contains an amount of radionuclide which has a radioactivity in the range of from 0.5 to 10 MBq, or from 1 to 10 MBq, or from 1 to 5 MBq, or from 5 to 10 MBq, or about 1, about 2, about 3, about 4, about 5, about 6, about 7, about 8, about 9 or about 10 MBq. In some embodiments, the radioactivity is measured at the timepoint immediately prior to administration of the dendrimer, i.e. immediately prior to use.

### Radionuclide Complexation Group

The radionuclide-containing moiety typically contains a radionuclide complexation group. Any suitable complexation group may be used. The complexation group provides functional moieties which can complex a radionuclide. Examples of such functional moieties include carboxylic acids, amines, amides, hydroxyl groups, thiol groups, ureas, thioureas, —N—OH groups, phosphate, and phosphinate groups. In some embodiments a complexation group which forms a chelate with the radionuclide is used. Examples of suitable complexation groups are provided in the table below:

| Ligand | Structure | Chemical Name |
|---|---|---|
| DOTA | 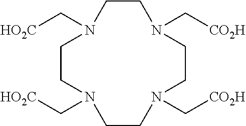 [SMILES: O=C(O)CN1CCN(CC(=O)O)CCN(CC(=O)O)CCN(CC(=O)O)CC1] | (1,4,7,10-Tetraazacyclodecane- N,N′,N″,N′′′-tetraacetic acid) |
| NOTA | 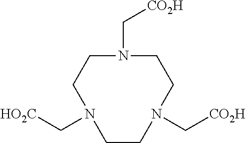 [SMILES: O=C(O)CN1CCN(CC(=O)O)CCN(CC(=O)O)CC1] | (1,4,7-Triazacyclononane-N,N′,N″- triacetic acid) |
| DTPA | 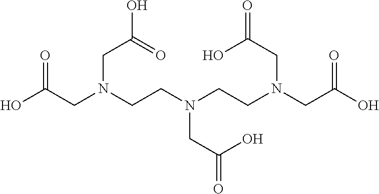 [SMILES: O=C(O)CN(CCN(CC(=O)O)CC(=O)O)CCN(CC(=O)O)CC(=O)O] | (Diethylenetriaminepentaacetic anhydride) |
| CHX-A DTPA | 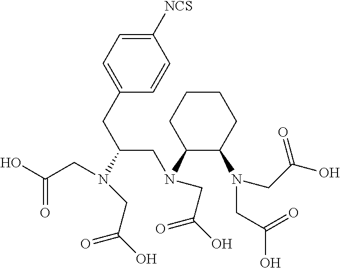 | [(R)-2-Amino-3-(4- isothiocyanatophenyl)propyl]-trans-(S,S)- cyclohexane-1,2-diamine-pentaacetic acid |
| Deferoxamine | 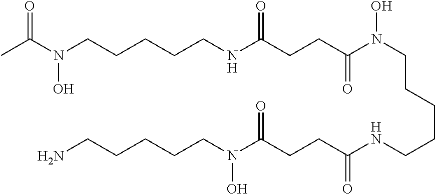 [SMILES: CC(=O)N(O)CCCCCNC(=O)CCC(=O)N(O)CCCCCNC(=O)CCC(=O)N(O)CCCCCN] | (N′-[5-[[4-[[5-(Acetylhydroxyamino) phenyl]amino]-1,4-dioxobutyl] hydroxyamino]pentyl]-N-(5- aminopentyl)-N-hydroxy-butanediamide) |
| TETA | 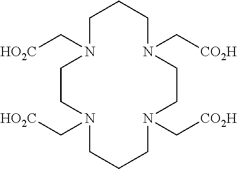 [SMILES: O=C(O)CN1CCCN(CC(=O)O)CCN(CC(=O)O)CCCN(CC(=O)O)CC1] | (1,4,8,11-tetraazacyclotetradecane-1,4,8,11- tetraacetic acid) |
| cb-TE2A | 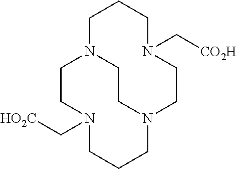 [SMILES: O=C(O)CN1CCCN2CCN(CCCN(CC(=O)O)CC2)CC1] | 1,4,8,11- Tetraazabicyclo[6.6.2]hexadecane-4,11- diacetic acid |
| AAZTA | 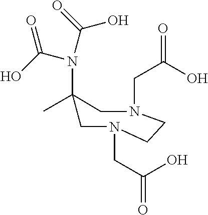 [SMILES: CC1(N(C(=O)O)C(=O)O)CN(CC(=O)O)CCN(CC(=O)O)C1] | 1,4-Bis(carboxymethyl)-6- [bis(carboxymethyl)]amino-6- methylperhydro-1,4-diazepine) |
| TRAP | 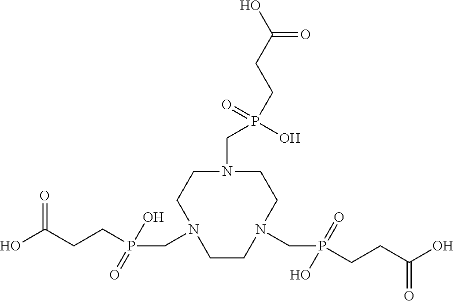 [SMILES: O=C(O)CCP(=O)(O)CN1CCN(CP(=O)(O)CCC(=O)O)CCN(CP(=O)(O)CCC(=O)O)CC1] | (1,4,7-Triazacyclononane phosphinic acid) |
| NOPO | 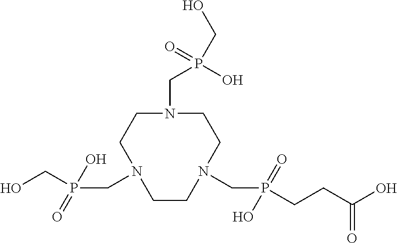 [SMILES: O=C(O)CCP(=O)(O)CN1CCN(CP(=O)(O)CO)CCN(CP(=O)(O)CO)CC1] | 1,4,7-Triazacyclononane-1,4- bis[methylene(hydroxymethyl)phosphinic acid]-7-[methylene(2- carboxyethyl)phosphinic acid] |
| HOPO | 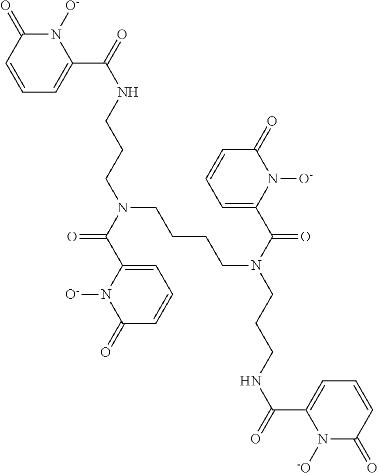 | 6-[(3-{[(1-Oxido-6-oxo-1,6-dihydro-2-pyri- dinyl)carbonyl]amino}propyl)(4-{[(1-oxido- 6-oxo-1,6-dihydro-2-pyridinyl)carbonyl](3- {[(1-oxido-6-oxo-1,6-dihydro-2-pyridinyl) carbonyl]amino}propyl)amino}butyl)car- bamoyl]-2-oxo-1(2H)-pyridinolate |
| NOGADA | 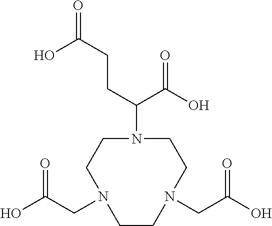 [SMILES: O=C(O)CCC(C(=O)O)N1CCN(CC(=O)O)CCN(CC(=O)O)CC1] | 2-[4,7-Bis(carboxymethyl)-1,4,7-triazonan- 1-yl]pentanedioic acid |
| HYNIC | 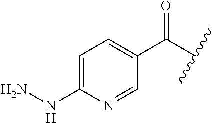 [SMILES: *C(=O)c1ccc(NN)nc1] | 6-Hydrazinonicotinic acid |
| MAG3 | 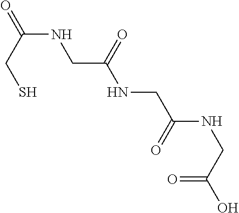 [SMILES: O=C(O)CNC(=O)CNC(=O)CNC(=O)CS] | Mertiatide |
| OPTT | 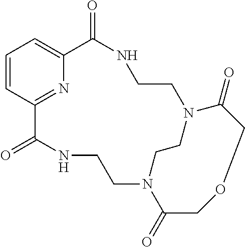 [SMILES: O=C1NCCN2CCN(CCNC(=O)c3cccc1n3)C(=O)COCC2=O] | 9-Oxa-3,6,12,15,21- pentaazatricyclo[15,3,2,1]trieicos- 1(21),17,19-triene-2,7,11,16-tetradione |
| TBPD | 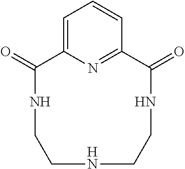 [SMILES: O=C1NCCNCCNC(=O)c2cccc1n2] | 3,6,9,15-Tetraazabicyclo[9.3.1]pentadeca- 1(15),11,13-triene-2,10-dione |
| TAME-Hex | 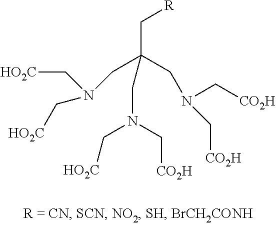 | 1,1,1-Tris(aminomethyl)ethane (basic skeleton) |
| DO3A | 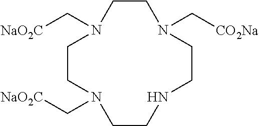 | 1,4,7,10-Tetraazacyclododecane-1,4,7- triacetic acid trisodium salt |
| TRAP | 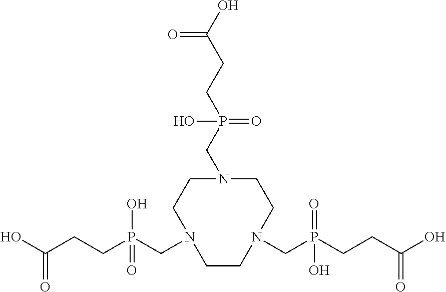 [SMILES: O=C(O)CCP(=O)(O)CN1CCN(CP(=O)(O)CCC(=O)O)CCN(CP(=O)(O)CCC(=O)O)CC1] | 1,4,7-Triazacyclononane-1,4,7-tris[(2- carboxyethyl)methylenephosphinic acid] |
| DATA | 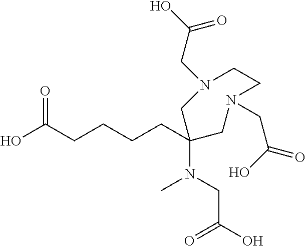 [SMILES: CN(CC(=O)O)C1(CCCCC(=O)O)CN(CC(=O)O)CCN(CC(=O)O)C1] | (2,2′-(6-((Carboxymethyl) amino)-1,4-diazepane-1,4-diyl)diacetc acid)) |
| NODAGA | 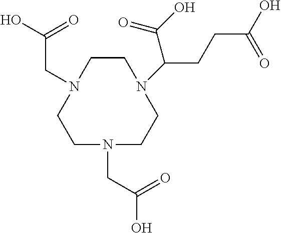 [SMILES: O=C(O)CCC(C(=O)O)N1CCN(CC(=O)O)CCN(CC(=O)O)CC1] | 2-[4,7-Bis(carboxymethyl)-1,4,7-triazonan- 1-yl]pentanedioic acid |
| DOTAGA | 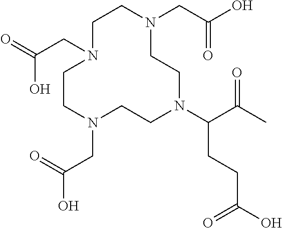 [SMILES: CC(=O)C(CCC(=O)O)N1CCN(CC(=O)O)CCN(CC(=O)O)CCN(CC(=O)O)CC1] | 1,4,7,10-Tetraazacyclododececane,1- (glutaric acid)-4,7,10-triacetic acid |
| PhenA | 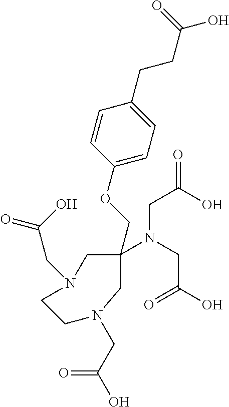 [SMILES: O=C(O)CCc1ccc(OCC2(N(CC(=O)O)CC(=O)O)CN(CC(=O)O)CCN(CC(=O)O)C2)cc1] | (2,2′-(6-(Bis(carboxymethyl)amino)-6-((4- (2-carboxyethyl)phenoxy)methyl)-1,4- diazepane-1,4-diyl)diacetic acid) |
| PCB- TE1A1P | 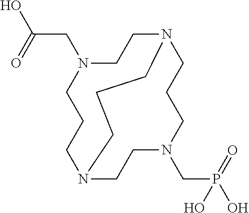 [SMILES: O=C(O)CN1CCCN2CCCN(CCCN(CP(=O)(O)O)CC2)CC1] | 11-Carboxymethyl-1,4,8,11- tetraazabicyclo[6.6.2]hexadecane-4- methanephosphonic acid |
| H3THP | 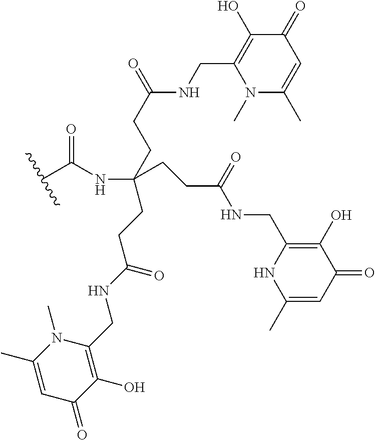 [SMILES: *C(=O)NC(CCC(=O)NCc1[nH]c(C)cc(=O)c1O)(CCC(=O)NCc1c(O)c(=O)cc(C)n1C)CCC(=O)NCc1c(O)c(=O)cc(C)n1C] | Tris(6-hydroxypyridin-2-ylmethyl)amine |
| DOTA- 4AMP | 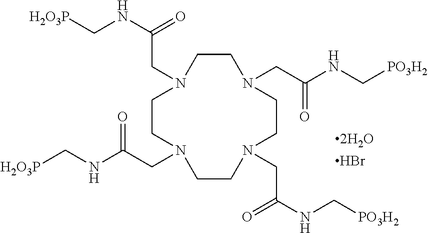 | 1,4,7,10-Tetraazacyclododecane-1,4,7,10- tetrakis(acetamido-methylenephosphonic acid) |
| NOTP | 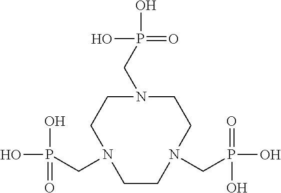 [SMILES: O=P(O)(O)CN1CCN(CP(=O)(O)O)CCN(CP(=O)(O)O)CC1] | 1,4,7-Triazacyclononane-1,4,7- tri(methylene phosphonic acid) |
| CB-Cyclam | 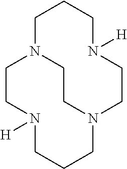 [SMILES: C1CNCCN2CCCNCCN(C1)CC2] | 1,4,8,11-Tetraazabicyclo[6.6.2]hexadecan |
| DiAmSar | 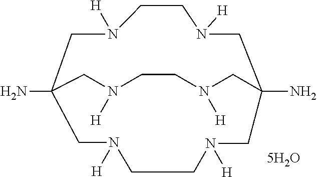 [SMILES: NC12CNCCNCC(N)(CNCCNC1)CNCCNC2] | 1,8-Diamino-3,6,10,13,16,19- hexaazabicyclo[6.6.6]-eicosane |
| DOTMA | 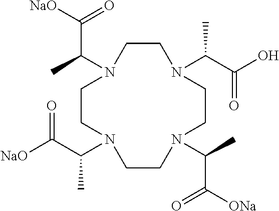 | (1R,4R,7R,10R)-α,α′,α″,α′′′-Tetramethyl- 1,4,7,10-tetraazacyclododecane-1,4,7,10- tetraacetic acid tetrasodium salt |
| DOTP | 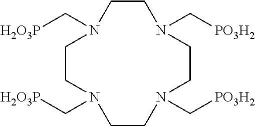 | 1,4,7,10-Tetraazacyclododecane-1,4,7,10- tetra(methylene phosphonic acid) |
| HBED | 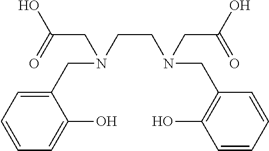 [SMILES: O=C(O)CN(CCN(CC(=O)O)Cc1ccccc1O)Cc1ccccc1O] | 2,2′-{1,2-Ethanediylbis[(2- hydroxybenzyl)imino]}diacetic acid |
| 6SS | 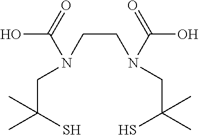 [SMILES: CC(C)(S)CN(CCN(CC(C)(C)S)C(=O)O)C(=O)O] | N,N′-Bis(2,2-dimethyl-2-mercaptoethyl) ethylenediamine-N,N′-diacetic acid |
| SarAr | 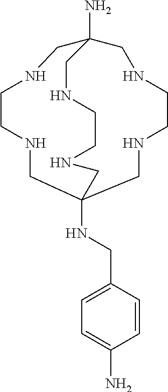 [SMILES: Nc1ccc(CNC23CNCCNCC(N)(CNCCNC2)CNCCNC3)cc1] | (1-N-(4-Aminobenzyl)-3,6,10,13,16,19- hexaazabicyclo[6.6.6]-eicosane-1,8- diamine) |
| MeCOSar | 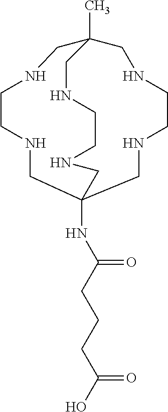 [SMILES: CC12CNCCNCC(NC(=O)CCCC(=O)O)(CNCCNC1)CNCCNC2] | 5-(8-Methyl-3,6,10,13,16,19-hexaaza- bicyclo[6.6.6]icosan-1-ylamino)-5- oxopentanoic acid |
| Sar (sarcophagine) | 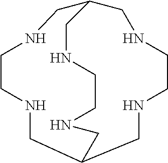 [SMILES: C1CNCC2CNCCNCC(CN1)CNCCNC2] | 3,6,10,13,16,19- hexaazabicyclo(6,6,6)icosane |
| H₂KTSM | 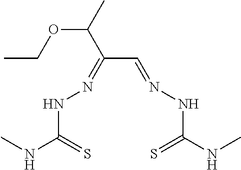 | 3-Ethoxy-2-oxobutyraldehyde-bis(N⁴- methylthiosemicarbazone) |
| H₂ATSM | 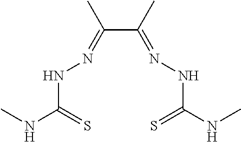 | Diacetyl-2-(N⁴-methyl-3- thiosemicarbazone)-3-(N⁴-amino-3- thiosemicarbazone) |
| TCMC | 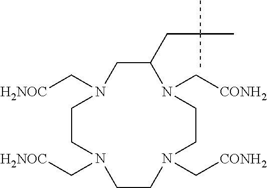 | 1,4,7,10-Tetraaza-1,4,7,10-tetra(2- carbamoylmethyl)cyclododecane |

In some embodiments, the complexation group is DOTA, NOTA, DTPA, sarcophagine or DFO. In some embodiments, the complexation group is DOTA, NOTA, DTPA or DFO.

In some embodiments, the complexation group is a DOTA-containing group having the structure

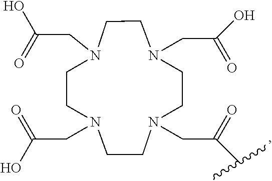 [SMILES: *C(=O)CN1CCN(CC(=O)O)CCN(CC(=O)O)CCN(CC(=O)O)CC1]

and wherein the DOTA-containing group is attached to the conjugate.

In some embodiments, the complexation group is a NOTA-containing group having the structure

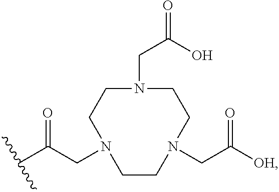 [SMILES: *C(=O)CN1CCN(CC(=O)O)CCN(CC(=O)O)CC1]

and wherein the NOTA-containing group is attached to the conjugate.

In some embodiments, the complexation group is a DTPA-containing group having the structure

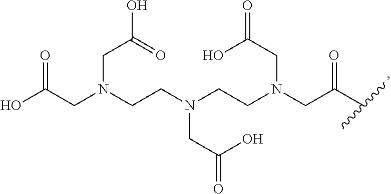 [SMILES: *C(=O)CN(CCN(CCN(CC(=O)O)CC(=O)O)CC(=O)O)CC(=O)O]

wherein the DTPA-containing group is attached to the conjugate.

In some embodiments, the complexation group is a DFO-containing group having the structure

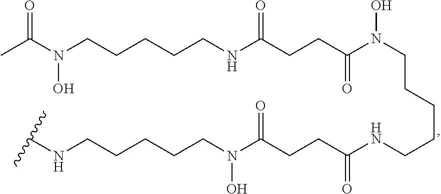 [SMILES: *NCCCCCN(O)C(=O)CCC(=O)NCCCCCN(O)C(=O)CCC(=O)NCCCCCN(O)C(C)=O]

wherein the DFO-containing group is attached to the conjugate.

In some embodiments, the complexation group is a sarcophagine-containing group having the structure

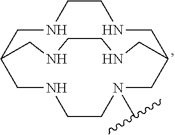 [SMILES: *N1CCNCC2CNCCNCC(CNCCNC2)C1]

wherein the sarcophagine-containing group is attached to the conjugate.

The first terminal group is attached to an outermost building unit, e.g. via a nitrogen atom of an outermost building unit where the building units are lysine residues or analogues thereof. In some embodiments, where a complexation group comprises a group which is suitable for direct reaction with an outermost building unit, the complexation group may be reacted directly with the building unit. In other embodiments, a loading group may be utilised to load the complexation group on to the dendrimer, i.e. a group which at a first end is covalently attached to the complexation group, and which at a second end has a functional group suitable for reaction with a functional group present on an outermost building unit (e.g. where the first terminal group is attached via a nitrogen atom of an outermost building unit. For example, the loading group may have a functional group which is suitable for reaction with an amino group.

To form the attachment between the outermost building unit and the first terminal group, a reaction may be carried out between a suitable complexation precursor groups and a dendrimeric intermediate having functional groups (e.g. amine groups) available for reaction. In some embodiments, the complexation precursor is a DOTA-containing, NOTA-containing, DTPA-containing, sarcophagine-containing or DFO-containing group. Examples of suitable complexation precursor groups include the following:

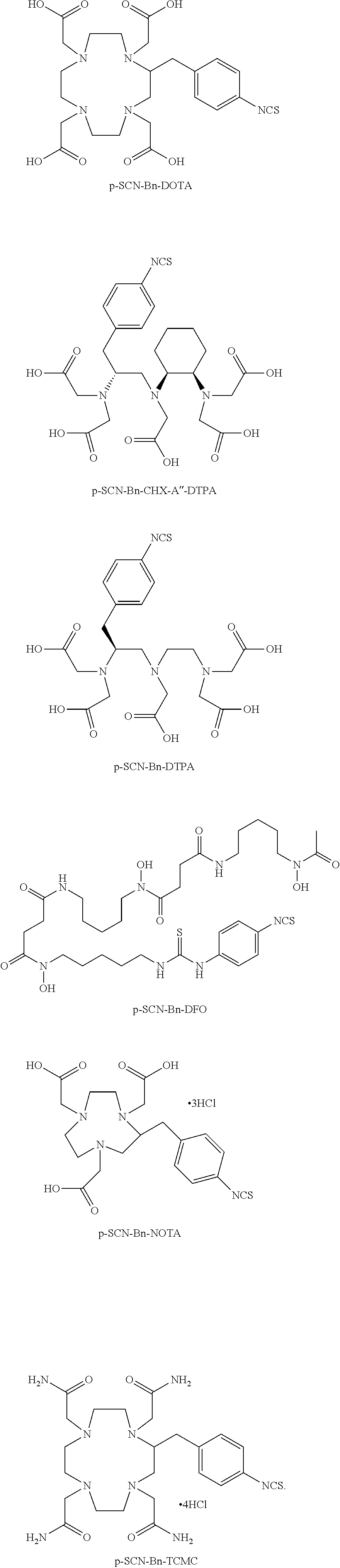

The above such groups can react with an amine group present on an outermost building unit to form a thiourea-linked first terminal group.

### Second Terminal Group

The dendrimer comprises a plurality of second terminal groups (T2) each comprising a pharmacokinetic-modifying moiety, i.e. a moiety that can modify or modulate the pharmacokinetic profile of the dendrimer. The pharmacokinetic modifying moiety may modulate the absorption, distribution, metabolism, excretion and/or toxicity of the dendrimer. The pharmacokinetic modifying moiety (T2) may change the solubility profile of the dendrimer, either increasing or decreasing the solubility of the dendrimer in a pharmaceutically acceptable carrier. The pharmacokinetic modifying moiety (T2) may for example reduce clearance of the dendrimer.

Where the dendrimer comprises a third terminal group comprising a pharmaceutically active agent, the pharmacokinetic modifying moiety (T2) may influence the rate of release of the pharmaceutically active agent, either by slowing or increasing the rate in which the active agent is released from the dendrimer by either chemical (e.g., hydrolysis) or enzymatic degradation pathways. The pharmacokinetic modifying moiety (T2) may assist the dendrimer in delivering the pharmaceutically active agent to specific tissues (e.g. tumours).

In some embodiments, the pharmacokinetic-modifying moiety is a polyethylene glycol (PEG) group or a polyethyloxazoline (PEOX) group.

In some embodiments the second terminal group comprises a PEG group. A PEG group is a polyethylene glycol group, i.e. a group comprising repeat units of the formula —CH₂CH₂O—. PEG materials used to produce the dendrimer of the present disclosure typically contain a mixture of PEGs having some variance in molecular weight (i.e., ±10%), and therefore, where a molecular weight is specified, it is typically an approximation of the average molecular weight of the PEG composition. For example, the term “PEG_{˜2100}” refers to polyethylene glycol having an average molecular weight of approximately 2100 Daltons, i.e. ± approximately 10% (PEG₁₈₉₀ to PEG₂₃₁₀). The term “PEG_{˜2300}” refers to polyethylene glycol having an average molecular weight of approximately 2300 Daltons, i.e. ± approximately 10% (PEG₂₀₇₀ to PEG₂₅₃₀). Three methods are commonly used to calculate MW averages: number average, weight average, and z-average molecular weights. As used herein, the phrase “molecular weight” is intended to refer to the weight-average molecular weight which can be measured using techniques well-known in the art including, but not limited to, NMR, mass spectrometry, matrix-assisted laser desorption ionization time of flight (MALDI-TOF), gel permeation chromatography or other liquid chromatography techniques, light scattering techniques, ultracentrifugation and viscometry.

In some embodiments, the second terminal groups comprise PEG groups having an average molecular weight of between about 200 and 5000 Daltons. In some embodiments, the second terminal groups comprise PEG groups having an average molecular weight of at least 500 Daltons, or at least 750 Daltons. In some embodiments, the second terminal groups comprise PEG groups having an average molecular weight in the range of from 200 to 4000 Daltons, or from 500 to 3000 Daltons, or from 500 to 2500 Daltons, or from 1500 to 2500 Daltons. In some embodiments, the second terminal groups comprise PEG groups having an average molecular weight in the range of from 220 to 2500 Da, or from 570 to 2500 Daltons, or from 220 to 1100 Daltons, or from 570 to 1100 Daltons, or from 1000 to 5500 Daltons, or from 1000 to 2500 Daltons, or from 1000 to 2300 Daltons. In some embodiments, the second terminal groups comprise PEG groups having an average molecular weight in the range of from 1900 to 2300 Daltons. In some embodiments, the second terminal groups comprise PEG groups having an average molecular weight in the range of from 2100 to 2500 Daltons. In some embodiments, the second terminal groups comprise PEG groups having an average molecular weight in the range of from 2400 to 2800 Daltons. In some embodiments, the second terminal groups comprise PEG groups having an average molecular weight of about 1900, about 2000, about 2100, about 2200, about 2300, about 2400, about 2500, about 2600, about 2700 or about 2800 Daltons.

In some embodiments, the PEG group has a polydispersity index (PDI) of between about 1.00 and about 1.50, between about 1.00 and about 1.25, or between about 1.00 and about 1.10. In some embodiments, the PEG group has a polydispersity index (PDI) of about 1.05. The term “polydispersity index” refers to a measure of the distribution of molecular mass in a given polymer sample. The polydispersity index (PDI) is equal to the weight average molecular weight (Mw) divided by the number average molecular weight (M) and indicates the distribution of individual molecular masses in a batch of polymers. The polydispersity index (PDI) has a value equal to or greater than one, but as the polymer approaches uniform change length and average molecular weight, the polydispersity index (PDI) will be closer to one.

Where the second terminal groups comprise a PEG group, the PEG groups may be linear or branched. If desired, an end-capped PEG group may be used. In some embodiments, the PEG group is a methoxy-terminated PEG.

In some embodiments the second terminal group comprises a PEOX group. A PEOX group is a polyethyloxazoline group, i.e. a group comprising repeat units of the formula

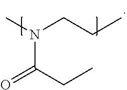

PEOX groups are so named since they can be produced by polymerisation of ethyloxazoline. PEOX materials used to produce the dendrimer of the present disclosure typically contain a mixture of PEOXs having some variance in molecular weight (i.e., +10%), and therefore, where a molecular weight is specified, it is typically an approximation of the average molecular weight of the PEOX composition. In some embodiments, the second terminal groups comprise PEOX groups having an average molecular weight of at least 750 Daltons, at least 1000 Daltons, or at least 1500 Daltons. In some embodiments, the second terminal groups comprise PEOX groups having an average molecular weight in the range of from 750 Daltons to 2500 Daltons, or from 1000 Daltons to 2000 Daltons. If desired, an end-capped PEOX group may be used. In some embodiments, the PEOX group is a methoxy-terminated PEOX.

The second terminal group may be attached to the outermost building unit via any suitable means. In some embodiments, where the second terminal group comprises a PEG group or PEOX group, a linking group is used to attach the PEG group or PEOX group to the outer building unit.

The second terminal groups are typically attached via use of a second terminal group precursor which contains a reactive group that is reactive with an amine group, such as a reactive acyl group (which can form an amide bond), or an aldehyde (which can form an amine group under reductive amination conditions).

In some embodiments, the second terminal groups each comprise a PEG group covalently attached to a PEG linking group (L1) via an ether linkage formed between a carbon atom present in the PEG group and an oxygen atom present in the PEG linking group, and each second terminal group is covalently attached to a building unit via an amide linkage formed between a nitrogen atom present in a building unit and the carbon atom of an acyl group present in the PEG linking group. In some embodiments, the second terminal groups are each

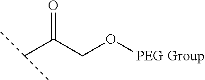

and wherein the PEG group is a methoxy-terminated PEG having an average molecular weight in tie range of from about 500 to 3000 Daltons, or from 2000 to 2700 Daltons.

In some embodiments, the second terminal groups each comprise a PEOX group covalently attached to a PEOX linking group (L1′) via a linkage formed between a nitrogen atom present in the PEOX group and a carbon atom present in the PEOX linking group, and each second terminal group is covalently attached to a building unit via an amide linkage formed between a nitrogen atom present in a building unit and the carbon atom of an acyl group present in the PEOX linking group. In some embodiments, the second terminal groups are each

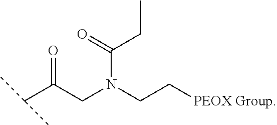

### Third Terminal Group

In some embodiments, the dendrimer comprises one or more third terminal groups (T3) attached to an outermost building unit, the third terminal group comprising a residue of a pharmaceutically active agent. Where the building units are lysine residues or analogues thereof, the third terminal group may for example be attached to the nitrogen atom of an outermost building unit. Incorporation of a pharmaceutically active agent into the dendrimer can provide improved therapeutic properties, and can lead to the same dendrimeric agent being capable of utilisation for both diagnostic/theranostic imaging, and for therapy of disease. For example, in the case of a subject who is suspected of having or who has been diagnosed as having a cancer, the dendrimer of the present disclosure may initially be administered and imaging of the relevant part(s) of the subject's body carried out, in order to diagnose the patient's condition by imaging and/or, where cancer is present, to determine the likely susceptibility of the cancer to a course of therapy with the dendrimer. In the case where the tumour is likely susceptible to treatment with dendrimer, a further course of the same dendrimer, or another dendrimer of the present disclosure, e.g. containing a different radionuclide, may for example then be administered to the subject.

### Pharmaceutically Active Agents

Any suitable pharmaceutically active agent may be conjugated to the dendrimer as the third terminal group, for example via a linking group. In some embodiments, the pharmaceutically active agent is an anti-cancer agent. In some embodiments, the anti-cancer agent is an anti-neoplastic drug that releases from the dendrimer to exert biological activity. In some embodiments, the anti-cancer agent is an ultratoxic agent. In some embodiments, the anti-cancer agent is an auristatin. In some embodiments, the anti-cancer agent is a maytansinoid. In some embodiments the anticancer agent is an alkylating agent, an anti-metabolite, vinca alkaloid, antibiotic, taxane, or topoisomerase inhibitor. In some embodiments, where the dendrimer comprises a pharmaceutically active agent, the anticancer agent is selected from the group consisting of a platinum contain moiety, an auristatin, a maytansinoid, a taxane, a topoisomerase inhibitor and a nucleoside analogue. In some embodiments, where the dendrimer comprises a pharmaceutically active agent, the pharmaceutically active agent is an anti-cancer agent, for example, an anti-cancer agent selected from the group consisting of cisplatin, carboplatin, oxaliplatin, temozolomide, docetaxel, cabazitaxel, paclitaxel, irinotecan, SN-38, camptothecin, topotecan, gemcitabine, barasertib, doxorubicin, cyclophosphamide, bleomycin, cisplatin, 5-fluorouracil, capecitabine, vincristine, dacarbazine, mitoxanthrone, teniposide, etoposide, aclarubicin, palbociclib, abiraterone acetate, lenalidomide, everolimus, and nilotinib. In some embodiments, where the dendrimer comprises a pharmaceutically active agent which is an anticancer agent, the anticancer agent is selected from the group consisting of cabazitaxel, docetaxel, SN-38 and gemcitabine.

In some embodiments, where the dendrimer comprises a pharmaceutically active agent which is an anticancer agent, the anticancer agent is a topoisomerase inhibitor. Topoisomerase inhibitors include, but are not limited to, camptothecin actives.

Camptothecin is a topoisomerase inhibitor having the structure:

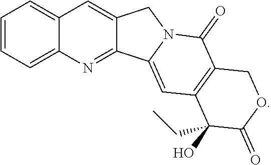 [SMILES: CC[C@@]1(O)C(=O)OCc2c1cc1n(c2=O)Cc2cc3ccccc3nc2-1]

A family of structurally-related compounds also having topoisomerase inhibitory activity have also been identified. In one embodiment, a camptothecin active is a compound having the substructure:

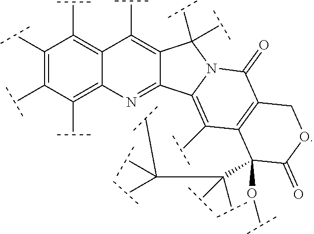

Examples of camptothecin actives (the residue of which may form part of the third terminal group) include SN-38, irinotecan (CPT-11), topotecan, silatecan, cositecan, exatecan, lurtotecan, gimatecan, belotecan and rubitecan. In some embodiments, the residue of a camptothecin active is attached to the diacyl linker through the C-10 or C-20 position. In some embodiments, the residue of a camptothecin active has the substructure:

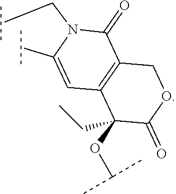

In some embodiments, the residue of a camptothecin active has the substructure:

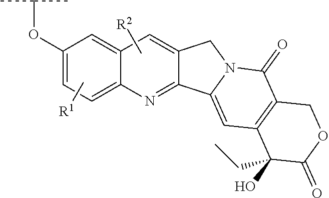

in which R¹ is selected from the group consisting of hydrogen, C₁₋₆ alkyl, —OR³, and —C₁₋₆ alkyl-N(R³)₂; R² is selected from the group consisting of hydrogen, C₁₋₆ alkyl, —OR³, and —C₁₋₆ alkyl-N(R³)₂; each R³ is independently selected from hydrogen and C₁₋₆ alkyl. In some embodiments, the third terminal group comprises a residue of a camptothecin active which is a residue of SN-38. SN-38 has the structure:

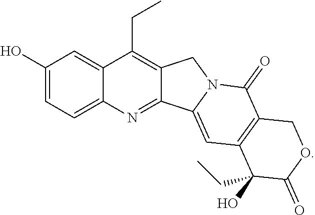 [SMILES: CCc1c2c(nc3ccc(O)cc13)-c1cc3c(c(=O)n1C2)COC(=O)[C@]3(O)CC]

In some embodiments, the residue of a camptothecin active is a residue of SN-38 which is attached to the diacyl linker through the C-10 or C-20 position. In some preferred embodiments the residue of SN-38 is

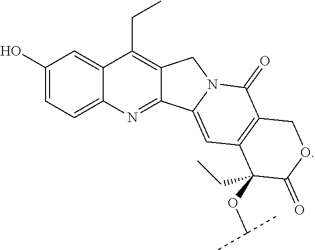

In other embodiments the residue of SN-38 is

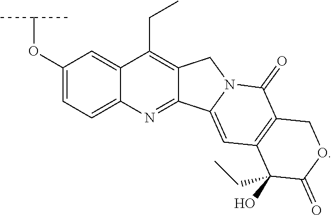

Upon in vivo administration, typically the dendrimer releases camptothecin active (e.g. SN-38).

In some embodiments, the pharmaceutically active agent is irinotecan.

In some embodiments, where the dendrimer comprises a pharmaceutically active agent which is an anticancer agent, the anticancer agent is a taxane. Taxane actives include paclitaxel, cabazitaxel and docetaxel. In some embodiments, the pharmaceutically active agent is paclitaxel. In some embodiments, the pharmaceutically active agent is cabazitaxel. In some embodiments, the pharmaceutically active agent is docetaxel. In some embodiments, the residue of a taxane active has the substructure:

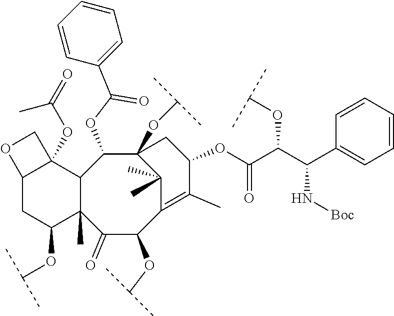

In some embodiments, the residue of a taxane active is a residue of cabazitaxel which is:

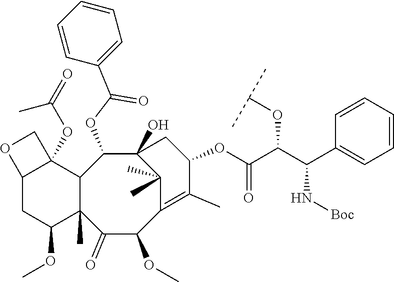

In some embodiments, the residue of a taxane active is a residue of docetaxel which is:

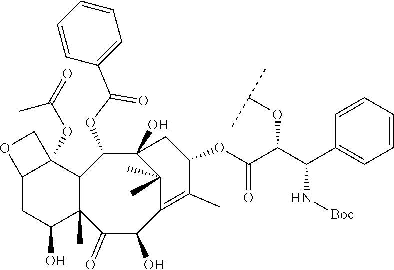

In some embodiments, the anti-cancer agent is selected from the group consisting of camptothecin actives and taxane actives.

In some embodiments, the anti-cancer agent is selected from the group consisting of cabazitaxel, docetaxel, and SN-38.

As used herein, the term “ultratoxic agent” refers to agents that exhibit highly potent chemotherapeutic properties, yet themselves are too toxic to administer alone as an anti-cancer agent. That is, an ultratoxic agent, although demonstrating chemotherapeutic properties, generally cannot be safely administered to a subject as the detrimental, toxic side-effects outweigh the chemotherapeutic benefit. In some embodiments, the ultratoxic has an in vitro IC₅₀ against a cancer cell line (e.g. SKBR3 and/or HEK293 cells and/or MCF7 cells) which is less than 100 nM, or less than 10 nM, or less than 5 nM, or less than 3 nM, or less than 2 nM, or less than 1 nM, or less than 0.5 nM. Ultratoxic agents include, for example, the dolastatins (e.g., dolastatin-10, dolastatin-15), auristatins (e.g., monomethyl auristatin-E, monomethyl auristatin-F), maytansinoids (e.g., maytansine, mertansine/emtansine (DM1, ravtansine (DM4)), calicheamicins (e.g., calicheamicin yl), esperamicins (e.g., esperamicin A1), and pyrrolobenzodiazepines (PDB) amongst others.

In some embodiments, the pharmaceutically active agent is an auristatin. In some embodiments, the pharmaceutically active agent is a monomethyl auristatin. In one embodiment, the pharmaceutically active agent is monomethyl auristatin E (MMAE). In one embodiment, the pharmaceutically active agent is monomethyl auristatin F (MMAF). Both MMAE and MMAF are understood to inhibit cell division by blocking the polymerisation of tubulin.

In some embodiments, the ultratoxic agent is a maytansinoid. In one embodiment, the ultratoxic agent is maytansine. In one embodiment, the ultratoxic agent is ansamitocin. In one embodiment, the ultratoxic agent is emtansine/mertansine (DM1). In one embodiment, the ultratoxic agent is ravtansine (DM4). The maytansinoids are understood to inhibit the assembly of microtubules by binding to tubulin.

In some embodiments, the pharmaceutically active agent is not an ultratoxic.

In some embodiments, the pharmaceutically active agent is a radio sensitiser.

In some embodiments the pharmaceutically active agent reduces DNA repair. In some embodiments the pharmaceutically active agent is selected from the group consisting of an agent targeting DNA-dependent protein kinase, checkpoint kinase 1, poly(ADP-ribose) polymerase such as olaparib, ataxia telangiectasia and/or Rad3-related protein such as AZD6738.

In some embodiments the pharmaceutically active agent is an immunotherapy agent. In some embodiments the immunotherapy agent selected from the group consisting of agents which block co-inhibitory molecules, CTLA-4, cytotoxic T-lymphocyte-associated protein 4, PD-1, programmed cell death protein 1, and/or which are checkpoint inhibitors.

In some embodiments the pharmaceutically active agent is a survival signalling inhibitor (proapoptotic). In some embodiments the agent is selected from the group consisting of an agent targeting: mTOR, mechanistic target of rapamycin; PI3K, phosphoinositide 3-kinase; and NF-κB, nuclear factor-kappa-B; In some embodiments the pharmaceutically active agent is an antihypoxic. In some embodiments the agent is selected from the group consisting of an agent targeting: CA9, carbonic anhydrase 9, HIF-1-α, hypoxia-inducible factor 1-alpha, and UPR, unfolded protein response. In some embodiments the agent is tirapazamine.

### Linkers

In some embodiments, where the dendrimer comprises a third terminal group (T3) comprising a residue of a pharmaceutically active agent, the residue of a pharmaceutically active agent is attached to an outermost building unit via a linker, for example a cleavable linker. Linker groups can be used for example to provide suitable groups for attaching a pharmaceutically active agent to the dendrimer, for example where available functionality in the pharmaceutically active agent is not suitable for direct attachment to a building unit. Linker groups can also or instead by used to facilitate controlled release of the pharmaceutically active agent from the dendrimeric scaffold, providing a therapeutically effective concentration and desirable pharmacokinetic profile of the pharmaceutically active agent for a suitable (e.g. prolonged) period of time.

A person skilled in the art will appreciate that any one of a variety of suitable linkers may be used. The linker should provide sufficient stability during systemic circulation, though allow for the rapid and efficient release of the pharmaceutically active agent (e.g. cytotoxic drug) in an active form at its site of action.

In some embodiments, the linker is a cleavable linker which, either itself or in conjunction with its linkage to the pharmaceutically active agent, comprises one or more of the following cleavable moieties: an ester group, a hydrazone group, an oxime group, an imine group or a disulphide group. In some embodiments, the linker is tumour environment cleavable, acid labile, reductive environment labile, hydrolytically labile or protease sensitive.

Chemically labile linkers include, but are not limited to, acid-labile linkers (i.e., hydrazones) and disulphide linkers. Enzymatically cleavable linkers include, but are not limited to, peptide linkers (e.g. those containing Val-Cit, or Phe-Lys groups), and β-glucuronide linkers. Peptide linkers, and their peptide bonds, are advantageously expected to have good serum stability, as lysosomal proteolytic enzymes have very low activities in blood. Both Val-Cit and Phe-Lys linkers are rapidly hydrolysed by Cathepsin B.

In some embodiments, the linker is an enzymatically-cleavable linker. For example, in some embodiments, the linker comprises amino acid residues which are capable of recognition and cleavage by an enzyme.

In some embodiments, the linker comprises a peptide group. In some embodiments, the linker comprises a valine-citrulline-paraaminobenzyl alcohol-containing group (Val-Cit-PAB), e.g. having the structure:

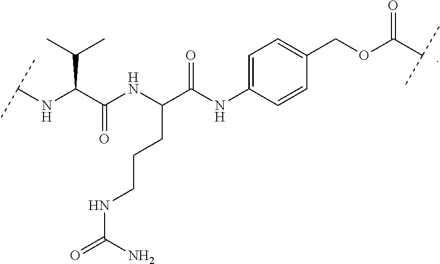

For example, the PAB group may be covalently attached to an amine group present on a therapeutic agent moiety via the carbonyl group, forming a carbamate linkage, and may be attached to an amine group present on an outer building unit via a diacyl linker which forms amide bonds with the valine amino group and the amine group present on the outer building unit.

In some embodiments, the linker comprises or consists of a glutaric acid-valine-citrulline-paraaminobenzyl alcohol group, e.g. having the structure:

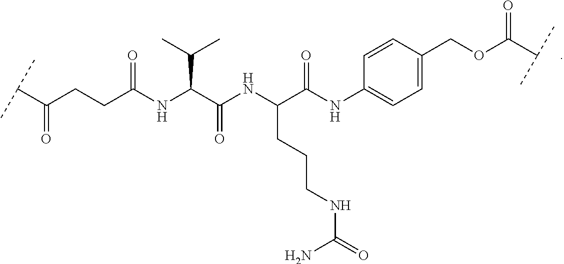

In some embodiments, the pharmaceutically active agent comprises a hydroxyl group, and the residue of the pharmaceutically active agent is attached to a linker via the oxygen atom of the hydroxyl group. This approach allows attachment to the linker via an ester group, and such ester groups have been found to be cleavable in vivo to release pharmaceutically active agent at a desirable rate.

In some embodiments, the core unit is formed from a core unit precursor comprising amino groups, the building units are lysine residues or analogues thereof, the pharmaceutically active agent comprises a hydroxyl group, the residue of the pharmaceutically active agent is attached via the oxygen atom of the hydroxyl group, and the cleavable linker is a diacyl linker, such that there is an ester linkage between the residue of the pharmaceutically active agent and the linker, and an amide linkage between the linker and a nitrogen atom present on an outermost building unit. In some embodiments, the pharmaceutically active agent comprises a hydroxyl group, the residue of the pharmaceutically active agent is attached via the oxygen atom of the hydroxyl group, and the cleavable linker is a diacyl linker group of formula

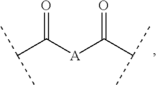

wherein A is a C₂-C₁₀ alkylene group which is optionally interrupted by O, S, S—S, NH, or N(Me), or in which A is a heterocycle selected from the group consisting of tetrahydrofuran, tetrahydrothiophene, pyrrolidine and N-methylpyrrolidine.

In some embodiments, the pharmaceutically active agent comprises a hydroxyl group, the residue of the pharmaceutically active agent is attached via the oxygen atom of the hydroxyl group, and the cleavable linker is a diacyl linker group of formula

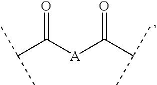

wherein A is a C₂-C₁₀ alkylene group which is interrupted by O, S, NH, or N(Me).

In some embodiments, the pharmaceutically active agent comprises a hydroxyl group, the residue of the pharmaceutically active agent is attached via the oxygen atom of the hydroxyl group, and the diacyl linker is

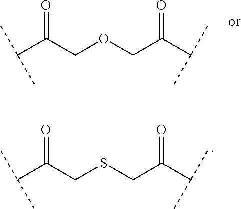

A specific type of cleavable linker is one which contains a disulphide moiety. Such linkers are susceptible to cleavage by glutathione. For example, a linker of this type may comprise two acyl groups linked via an alkyl chain interrupted by a disulphide moiety.

In some embodiments, the linker comprises an alkyl chain interrupted by a disulphide moiety, in which one or both of the carbon atoms which are next to the disulphide group are substituted by one or more methyl groups. For example, one of the carbon atoms next to the disulphide moiety may be substituted by a gem-dimethyl group, e.g. the linker may comprise the group:

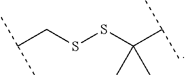

In some embodiments, the linker is

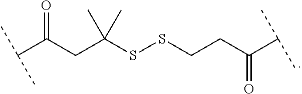

In some embodiments, each third terminal group (T3) is

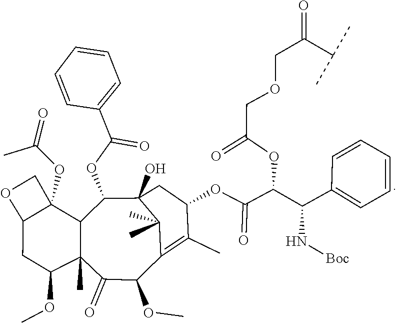

In some embodiments, each third terminal group (T3) is

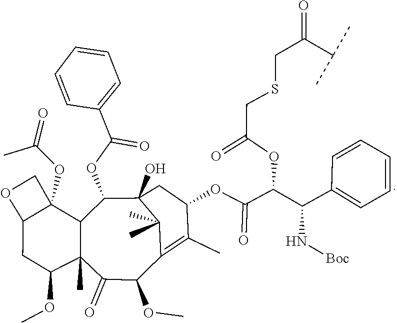

In some embodiments, each third terminal group (T3) is

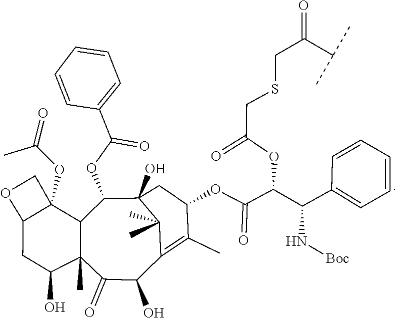

In some embodiments, each third terminal group (T3) is:

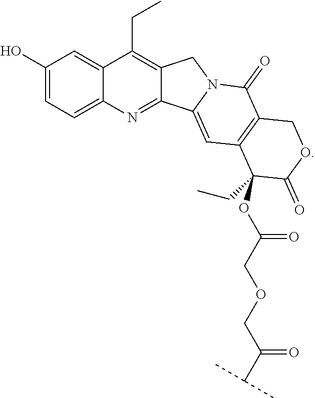

In some embodiments, the dendrimer comprises surface units comprising an outer building unit and a second terminal group of the formula:

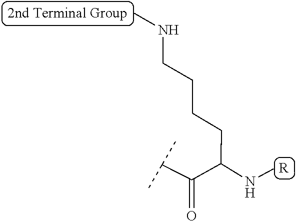

wherein R represents a first terminal group or a third terminal group.

In some embodiments, the dendrimers of the present disclosure have one or more first terminal groups attached to an outermost building unit, wherein each first terminal group comprises a radionuclide-containing moiety or a complexation group containing stable isotope (cold material); and one or more second terminal groups attached to a nitrogen atom of an outermost building unit, wherein each second terminal group comprises a pharmacokinetic-modifying moiety.

In some embodiments, the first terminal group is attached to the nitrogen atom of an outermost building unit, and the second terminal group is attached to the nitrogen atom of an outermost building unit. In some embodiments, where the dendrimer comprises a third terminal group comprising a residue of a pharmaceutically active agent, the third terminal group is attached to the nitrogen atom of an outermost building unit.

The dendrimers can thus be considered to have controlled stoichiometry and/or topology. For example, the dendrimers are typically produced using synthetic processes that allow for a high degree of control over the number and arrangement of first and second (and third) terminal groups present on the dendrimers. The dendrimers may be synthesised using orthogonal protecting groups to allow for conjugation of the terminal groups to the outer building unit in a predefined or controlled manner.

Advantageously, the dendrimers of the present disclosure can provide effective imaging and diagnostic properties despite containing relatively low loadings of radionuclide moiety. This is desirable both from a synthesis perspective, and since it provides for additional sites on the dendrimer building units to be available for conjugation to other useful moieties in the constructs, such as pharmaceutically active agents.

Accordingly, in some embodiments where the core unit is formed from a core unit precursor comprising amino groups and the building units are lysine residues or analogues thereof, less than 20%, less than 15%, less than 10%, less than 5%, or less than 1%, of the nitrogen atoms present in the outermost building units are attached to a first terminal group (i.e. a group comprising a radionuclide-containing moiety). In some embodiments, for example where the dendrimer has five generations of building units, from 1 to 5 (i.e. 1, 2, 3, 4 or 5) of the nitrogen atoms present in the outermost building units are attached to a first terminal group. In an embodiment of a composition of dendrimers, the average first terminal groups may be less than 1. In some embodiments, from 1 to 3 of the nitrogen atoms present in the outermost building units are attached to a first terminal group.

In some embodiments where the core unit is formed from a core unit precursor comprising amino groups and the building units are lysine residues or analogues thereof, at least 40% of the nitrogen atoms present in the outermost building units are each covalently attached to a second terminal group. In some embodiments, at least 45% of the nitrogen atoms present in the outer building units are each covalently attached to a second terminal group. In some embodiments, about 50% of the nitrogen atoms present in the outer building units are each covalently attached to a second terminal group. In some embodiments, for example where the dendrimer has five generations of building units, at least 25, 26, 27, 27, 29, 30, 31 or 32 of the nitrogen atoms present in the outermost building units are each covalently attached to a second terminal group.

As discussed above, the ability to achieve good therapeutic properties despite relatively low loading of radionuclide, provides for additional sites on the dendrimer outer building units to be available for conjugation to other useful moieties in the constructs, such as pharmaceutically active agents. Accordingly, in some embodiments where the core unit is formed from a core unit precursor comprising amino groups and the building units are lysine residues or analogues thereof, at least 25%, at least 30%, at least one third, at least 35%, or at least 45% of the nitrogen atoms present in the outer building units are each covalently attached to a third terminal group. In some embodiments, for example where the dendrimer has five generations of building units, at least 15, 16, 17, 18, 19, 20, 21, 22, 23, 24, 25, 26, 27, 28, 29, 30 or 31 of the nitrogen atoms present in the outermost building units are each covalently attached to a third terminal group.

In some embodiments where the core unit is formed from a core unit precursor comprising amino groups and the building units are lysine residues or analogues thereof, no more than one quarter of the nitrogen atoms present in the outermost generation of building units are unsubstituted. In some embodiments, the number of nitrogen atoms present in the outermost generation of building units that are substituted may be at least 70%, 75%, 80%, 85%, 90%, or 95%. In one embodiment, at least 80% of the nitrogen atoms present in the outermost generation of building units are substituted.

In some embodiments, the dendrimer comprises outermost building units which contain —NH₂ groups, for example where not all nitrogen atoms present on the outermost building units are attached to a first or second (or third) terminal group.

In some embodiments where the core unit is formed from a core unit precursor comprising amino groups and the building units are lysine residues or analogues thereof, for example where the dendrimer has five generations of building units, no more than 20 nitrogen atoms present in the outermost generation of building units are unsubstituted. In some embodiments, no more than 10 nitrogen atoms present in the outermost generation of building units are unsubstituted. In some embodiments, no more than 5 nitrogen atoms present in the outermost generation of building units are unsubstituted. In some embodiments, no more than 3 nitrogen atoms present in the outermost generation of building units are unsubstituted. In some embodiments, no more than 2 nitrogen atoms present in the outermost generation of building units are unsubstituted. In some embodiments, no more than 1 nitrogen atom present in the outermost generation of building units is unsubstituted. In some embodiments, substantially all of the nitrogen atoms present in the outermost generation of building units are substituted.

The number of first, second and, where present, third terminal groups which form part of the dendrimer can be varied, so as to tailor the properties of the dendrimer as desired. For example, the molar ratio of first terminal groups comprising a radionuclide-complexing moiety to third terminal groups comprising a pharmaceutically active agent can be varied. In some embodiments, the dendrimer has a molar ratio of complexation group to pharmaceutically active agent in the range of from 1:1 to 1:100, or from 1:1 to 1:50, or from 1:1 to 1:40, or from 1:1 to 1:30, or from 1:1 to 1:20, or from 1:1 to 1:10, or from 1:2 to 1:100, or from 1:2 to 1:50, or from 1:2 to 1:40, or from 1:2 to 1:30, or from 1:2 to 1:20, or from 1:2 to 1:10, or from 1:5 to 1:100, or from 1:5 to 1:50, or from 1:5 to 1:40, or from 1:5 to 1:40, or from 1:5 to 1:30, or from 1:5 to 1:20, or from 1:5 to 1:10, or from 1:10 to 1:100, or from 1:10 to 1:50, or from 1:10 to 1:40, or from 1:10 to 1:30, or from 1:10 to 1:20.

It will be appreciated that, in addition to the first, second and third terminal groups, further moieties may be attached to the dendrimer. For example, if desired, some nitrogen atoms present in the outermost generation of building units may be capped with a suitable capping group, e.g. which is substantially inert to further reaction under typical conditions utilised. An example of a suitable capping group is an acetyl group.

In some embodiments, an alpha-nitrogen atom of an outermost building unit is attached to a first terminal group (i.e. comprising a radionuclide-containing moiety).

In some embodiments, epsilon-nitrogen atoms of outermost building units are attached to second terminal groups (i.e. comprising a pharmacokinetic-modifying moiety).

In some embodiments, alpha-nitrogen atoms of outermost building units are attached to third terminal groups (i.e. comprising a residue of a pharmaceutically active agent).

In some embodiments an alpha-nitrogen atom of an outermost building unit is attached to a first terminal group, alpha-nitrogen atoms of outermost building units are attached to third terminal groups, and epsilon-nitrogen atoms of outermost building units are attached to second terminal groups.

It will be appreciated that when the first terminal group comprises complexation group and a radionuclide-containing moiety, other In some embodiments, the dendrimer is any of the Example dendrimers as described herein.

### Dendrimer Compositions

In some embodiments, the dendrimer is presented as a composition, preferably a pharmaceutical composition. Accordingly, there is also provided a composition comprising a plurality of conjugates as described herein. In some embodiments, the composition is a pharmaceutical composition (i.e. a composition suitable for administration to a subject for therapeutic or diagnostic purposes) comprising the dendrimer and a pharmaceutically acceptable excipient.

It will be appreciated that there may be some variation in the molecular composition between the dendrimers present in a given composition, as a result of the nature of the synthetic process for producing the dendrimers. For example, as discussed above one or more synthetic steps used to produce a dendrimer may not proceed fully to completion, which may result in the presence of dendrimers which do not all comprise the same number of first terminal groups or second terminal groups, or which contain incomplete generations of building units.

Accordingly, in one embodiment there is provided a composition comprising a plurality of dendrimers or salts thereof, wherein at least some of the dendrimers are as defined herein, and wherein the mean number of first terminal groups per dendrimer in the composition is in the range of from 0.2 to 8, and the mean number of second terminal groups per dendrimer in the composition is in the range of from 10 to 32.

For example, the degree of labelling required to achieve good imaging or therapeutic efficacy may be relatively low, potentially even requiring less than one radiolabelled group per dendrimer in some instances. However, in some embodiments, the mean number of first terminal groups per dendrimer in the composition is in the range of from 1 to 5, and the mean number of second terminal groups per dendrimer in the composition is in the range of from 10 to 32.

In some embodiments, the composition comprises dendrimers having a third terminal group comprising a residue of a pharmaceutically active agent, and the mean number of third terminal group per dendrimer in the composition is in the range of from 10 to 31.

In some embodiments, the composition is a pharmaceutical composition, and the composition comprises a pharmaceutically acceptable excipient.

In some embodiments, at least 50%, at least 60%, at least 70%, at least 80%, at least 90%, or at least 95% of the dendrimers contain a first terminal group.

In some embodiments, at least 50%, at least 60%, at least 70%, at least 80%, at least 90%, or at least 95% of the dendrimers contain a second terminal group.

In some embodiments, at least 50%, at least 60%, at least 70%, at least 80%, at least 90%, or at least 95% of the dendrimers contain a third terminal group.

In some embodiments, at least 50% of the dendrimers contain at least one first terminal group.

In some embodiments, at least 75% of the dendrimers contain at least 26, at least 28, or at least 30 second terminal groups.

In some embodiments, at least 75% of the dendrimers contain at least 20, at least 22, at least 24, at least 26 or at least 28 third terminal groups comprising a residue of a pharmaceutically active agent.

As discussed above, the present disclosure provides pharmaceutical formulations or compositions, both for veterinary and for human medical use, which comprise the dendrimers of the present disclosure or a pharmaceutically acceptable salt thereof, with one or more pharmaceutically acceptable carriers, and optionally any other therapeutic ingredients, stabilisers, or the like. The carrier(s) must be pharmaceutically acceptable in the sense of being compatible with the other ingredients of the formulation and not unduly deleterious to the recipient thereof.

The compositions of the present disclosure may also include polymeric excipients/additives or carriers, e.g., polyvinylpyrrolidones, derivatised celluloses such as hydroxymethylcellulose, hydroxyethylcellulose, and hydroxypropylmethylcellulose, ficolls (a polymeric sugar), hydroxyethylstarch (HES), dextrates (e.g., cyclodextrins, such as 2-hydroxypropyl-β-cyclodextrin and sulfobutylether-β-cyclodextrin), polyethylene glycols, and pectin. The compositions may further include diluents, buffers, citrate, trehalose, binders, disintegrants, thickeners, lubricants, preservatives (including antioxidants), inorganic salts (e.g., sodium chloride), antimicrobial agents (e.g., benzalkonium chloride), sweeteners, antistatic agents, sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions according to the present disclosure are listed in “Remington: The Science & Practice of Pharmacy”, 19.sup.th ed., Williams & Williams, (1995), and in the “Physician's Desk Reference”, 52.sup.nd ed., Medical Economics, Montvale, N.J. (1998), and in “Handbook of Pharmaceutical Excipients”, Third Ed., Ed. A. H. Kibbe, Pharmaceutical Press, 2000.

The conjugates of the present disclosure may be formulated in compositions including those suitable for administration by any suitable route, including for example by parenteral (including intraperitoneal, intravenous, subcutaneous, or intramuscular injection) administration.

administration. The dendrimers of the present disclosure may be formulated in a composition suitable for administration for diagnostic and/or theranostic purposes.

The compositions may conveniently be presented in unit dosage form and may be prepared by any of the methods well known in the art of pharmacy. All methods include the step of bringing the dendrimer into association with a carrier that constitutes one or more accessory ingredients. In general, the compositions are prepared by bringing the dendrimer into association with a liquid carrier to form a solution or a suspension, or alternatively, bring the dendrimer into association with formulation components suitable for forming a solid, optionally a particulate product, and then, if warranted, shaping the product into a desired delivery form. Solid formulations of the present disclosure, when particulate, will typically comprise particles with sizes ranging from about 1 nanometer to about 500 microns. In general, for solid formulations intended for intravenous administration, particles will typically range from about 1 nm to about 10 microns in diameter. The composition may contain dendrimer of the present disclosure that are nanoparticulate having a particulate diameter of below 1000 nm, for example, between 5 and 1000 nm, especially 5 and 500 nm, more especially 5 to 400 nm, such as 5 to 50 nm and especially between 5 and 20 nm. In one example, the composition contains dendrimers with a mean size of between 5 and 20 nm. In some embodiments, the dendrimer is polydispersed in the composition, with PDI of between 1.01 and 1.8, especially between 1.01 and 1.5, and more especially between 1.01 and 1.2. In one example, the dendrimer is monodispersed in the composition.

In some preferred embodiments, the composition is formulated for parenteral delivery. For example, in one embodiment, the formulation may be a sterile, lyophilized composition that is suitable for reconstitution in an aqueous vehicle prior to injection.

In one embodiment, a formulation suitable for parenteral administration conveniently comprises a sterile aqueous preparation of the dendrimer, which may for example be formulated to be isotonic with the blood of the recipient.

In some embodiments, the composition is formulated for intertumoural delivery. Other suitable means of delivery may also be used. For example, in some embodiments delivery may be by lavage or aerosol. In one embodiment the composition is formulated for intraperitoneal delivery, and is for treatment of cancers in the peritoneal cavity, which include malignant epithelial tumors (e.g., ovarian cancer), and peritoneal carcinomatosis (e.g. gastrointestinal especially colorectal, gastric, gynaecologic cancers, and primary peritoneal neoplasms).

Pharmaceutical formulations are also provided which are suitable for administration as an aerosol, by inhalation. These formulations comprise a solution or suspension of the desired dendrimer or a salt thereof. The desired formulation may be placed in a small chamber and nebulized. Nebulization may be accomplished by compressed air or by ultrasonic energy to form a plurality of liquid droplets or solid particles comprising the dendrimers or salts thereof.

As discussed below, the dendrimers of the present disclosure may for example be administered in combination with one or more additional pharmaceutically active agents. In some embodiments, the dendrimer is provided in combination with a further active. In some embodiments, a composition is provided which comprises a dendrimer as defined herein or a pharmaceutically acceptable salt thereof, one or more pharmaceutically acceptable carriers, and one or more additional pharmaceutically active agents, e.g. an additional anti-cancer/oncology agent, such as a small molecule cytotoxic, a checkpoint inhibitor, or an antibody therapy. Not only can the dendrimers of the present disclosure be administered with other chemotherapy drugs but may also be administered in combination with other medications such as corticosteroids, anti-histamines, analgesics and drugs that aid in recovery or protect from hematotoxicity, for example, cytokines.

In some embodiments, the composition is formulated for parenteral infusion as part of a chemotherapy regimen.

### Diagnostic and Therapeutic Applications of Dendrimers

The dendrimers as described herein according to any aspects, embodiments or examples thereof, can be used in various diagnostic and therapeutic applications. The dendrimers as described herein can be used as sole diagnostic agent, such as an imaging agent, or as a dual diagnostic and therapeutic agent. Examples of the diagnostic and/or therapeutic applications include imaging, theranostics, companion diagnostic-therapeutic, monitoring disease progression, evaluating efficacy of therapy, determining patient group outcomes, and developing treatment regimes for specific patients or patient groups.

In one embodiment, there is provided a method of determining whether a subject has a cancer. A first step of the method may comprise administering to a subject a dendrimer or a pharmaceutical composition as described herein according to any aspects, embodiments or examples thereof. A second step of the method may comprise carrying out imaging on the subject's body or a part thereof. A third step of the method may comprise determining whether the subject has a cancer based on the imaging results.

In another embodiment, there is provided a method of imaging a cancer in a subject. A first step of the method may comprise administering to a subject having a cancer a dendrimer or a pharmaceutical composition as described herein according to any aspects, embodiments or examples thereof. A second step of the method may comprise carrying out imaging on the subject's body or a part thereof.

In another embodiment, there is provided a method of determining the progression of a cancer in a subject. A first step may comprise administering to a subject having a cancer a first amount of a dendrimer or a pharmaceutical composition as described herein according to any aspects, embodiments or examples thereof. A second step of the method may comprise carrying out an imaging step on the subject's body or a part thereof. A third step of the method may comprise subsequently administering to the subject a second amount of a dendrimer or a pharmaceutical composition as described herein according to any aspects, embodiments or examples thereof. A fourth step of the method may comprise carrying out a second imaging step on the subject's body or a part thereof. A fifth step of the method may comprise determining whether the cancer has progressed based on the first and second imaging results.

In another embodiment, there is provided a method of determining an appropriate therapy for a subject having a cancer. A first step of the method may comprise administering to the subject a dendrimer or a pharmaceutical composition as described herein according to any aspects, embodiments or examples thereof. A second step of the method may comprise carrying out imaging on the subject's body or a part thereof. A third step of the method may comprise determining if the imaging results indicate susceptibility of the cancer to treatment with a therapy, and subsequently as a further step administering the therapy to the subject.

In another embodiment, there is provided a method of determining the effectiveness of a cancer therapy administered to a subject having a cancer. A first step of the method may comprise administering to the subject a first amount of a dendrimer or a pharmaceutical composition as described herein according to any aspects, embodiments or examples thereof. A second step of the method may comprise carrying out a first imaging step on the subject's body or a part thereof. A third step may comprise administering to the subject a cancer therapy. A fourth step may comprise subsequently administering to the subject a second amount of a dendrimer or a pharmaceutical composition as described herein according to any aspects, embodiments or examples thereof. A fifth step may comprise carrying out a second imaging step on the subject's body or a part thereof. A sixth step may comprise determining the effectiveness of the cancer therapy based on the first and second imaging results.

The imaging as described herein, including for any of the above embodiments, may be PET imaging. In another embodiment, the imaging is, at least one of PET-MRI, SPECT, SPECT-CT, CT, scintography and PET-CT imaging.

The therapy may involve a dendrimer or a composition as described herein according to any aspects, embodiments or examples thereof.

As well as having use as diagnostic and theranostic imaging agents, the dendrimers of the present disclosure may be useful in the treatment of conditions such as cancers. Accordingly, there is also provided a dendrimer or pharmaceutical composition as described herein for use in therapy, and more specifically for use in therapy of cancer. In some embodiments, the dendrimer is used in a method of treating or preventing cancer, for example for suppressing the growth of a tumour. In some embodiments the dendrimer is for use in the treatment of cancer. There is also provided a method of treating cancer comprising administering to a subject in need thereof a therapeutically effective amount of the dendrimer. There is also provided use of a dendrimer as defined herein, or of a composition as defined herein, in the manufacture of a medicament for the treatment of cancer.

In some embodiments, the cancer is a solid tumour. The cancer may be a primary or metastatic tumour. In some embodiments the cancer is a primary tumour. In some embodiments the cancer is a metastatic tumour.

In some embodiments, the cancer is selected from the group consisting of colorectal cancer, pancreatic, cancer, breast cancer, ovarian cancer, prostate cancer, lung cancer and cervical cancer. In some embodiments, the cancer is prostate cancer, pancreatic cancer, gastrointestinal cancer, stomach cancer, lung cancer, uterine cancer, breast cancer, brain cancer or ovarian cancer. In some embodiments the cancer is prostate cancer, pancreatic cancer, breast cancer or brain cancer. In some embodiments, the cancer is selected from the group consisting of prostate cancer, brain cancers, breast cancers, testicular cancers, ovarian cancers, stomach cancers, adenocarcinomas of the lung, gastric cancers, pancreatic cancers, salivary duct carcinomas, oesophageal cancers, and uterine cancers (e.g., uterine serious endometrial carcinoma).

In some embodiments, the cancer is selected from the group consisting of colorectal cancer, stomach cancer, pancreas cancer, prostate cancer and breast cancer.

In some embodiments, the cancer is brain cancer. Brain cancers include, but are not limited to, glioblastoma, meningioma, pituitary, nerve sheath, astrocytoma, oligodendroglioma, ependymoma, medulloblastoma, or craniopharyngioma. The brain cancer may be a glioblastoma, meningioma, pituitary, nerve sheath, astrocytoma, oligodendroglioma, ependymoma, medulloblastoma, or craniopharyngioma. In one particular embodiment, the brain cancer is a glioblastoma. In some embodiments, the brain cancer is meningioma. In some embodiments, the brain cancer is pituitary. In some embodiments, the brain cancer is nerve sheath. In some embodiments, the brain cancer is astrocytoma. In some embodiments, the brain cancer is oligodendroglioma. In some embodiments, the brain cancer is ependymoma. In some embodiments, the brain cancer is medulloblastoma. In some embodiments, the brain cancer is craniopharyngioma. In some embodiments, the cancer is prostate cancer. In some embodiments, the cancer is breast cancer. In some embodiments, the cancer is testicular cancer. In some embodiments, the cancer is ovarian cancer. In some embodiments, the cancer is stomach cancer. In some embodiments, the cancer is adenocarcinoma of the lung. In some embodiments, the cancer is gastric cancer. In some embodiments, the cancer is pancreatic cancer. In some embodiments, the cancer is salivary duct carcinoma. In some embodiments, the cancer is oesophageal cancer. In some embodiments, the cancer is uterine cancer.

The dendrimer may be administered by any suitable route, including for example, intravenously. In some embodiments, the dendrimer is delivered as an IV bolus. In some embodiments the dendrimer is administered IV over a time a period in the range of from 0.5 to 60 minutes, or in the range of from 0.5 to 30 minutes, or in the range of from 0.5 to 15 minutes, or in the range of from 0.5 to 5 minutes. In another example, the dendrimer may be administered intraperitoneally. The route of administration may for example be targeted to the disease or disorder which the subject has. For example, in some embodiments the disease or disorder may be an intra-abdominal malignancy such as a gynecological or gastrointestinal cancer, and the conjugate may be administered intraperitoneally. In some embodiments the dendrimer may be for treatment of a cancer of the peritoneal cavity, such as a malignant epithelial tumors (e.g., ovarian cancer) or peritoneal carcinomatosis (e.g. gastrointestinal especially colorectal, gastric, gynecologic cancers, and primary peritoneal neoplasms), and the dendrimer is administered intraperitoneally.

Where the dendrimer comprises a third terminal group which is a further pharmaceutically active agent, in some embodiments, the amount of dendrimer administered is sufficient to deliver between 2 and 100 mg of active agent/m², between 2 and 50 mg of active agent/m², between 2 and 40 mg of active agent/m², between 2 and 30 mg of active agent/m², between 2 and 25 mg of active agent/m², between 2 and 20 mg of active agent/m², between 5 and 50 mg of active agent/m², between 10 to 40 mg of active agent/m² between 15 and 35 mg of active agent/m², between 10 and 20 mg/m², between 20 and 30 mg/m², or between 25 and 35 mg of active agent/m². A dose of active agent of 10 mg/kg in a mouse should be approximately equivalent to a human dose of 30 mg/m² (FDA guidance 2005). (To convert human mg/kg dose to mg/m², the figure may be multiplied by 37, FDA guidance 2005).

In some embodiments, a therapeutically effective amount of the dendrimer is administered to a subject in need thereof at a predetermined frequency. In some embodiments, the dendrimer is administered to a subject in need thereof according to a dosage regimen in which the dendrimer is administered once per one to four weeks. In some embodiments, the dendrimer is administered to a subject in need thereof according to a dosage regimen in which the dendrimer is administered once per three to four weeks.

As discussed above, a therapeutically effective amount of the dendrimer is administered. For example, in some embodiments when administered, a dose of dendrimer may be administered which provides an amount of radioactivity in the range of up to 50 GBq, from 1 to 20 GBq, or from 1 to 10 GBq. In some embodiments, when administered, a dose of dendrimer is administered which provides an amount of radioactivity in the range of from 0.1 to 10 MBq, from 0.1 to 5 MBq, from 0.1 to 2 MBq, from 0.1 to 1 MBq, from 0.5 to 10 MBq, from 1 to 10 MBq, from 1 to 5 MBq, from 5 to 10 MBq, or about 1, about 2, about 3, about 4, about 5, about 6, about 7, about 8, about 9 or about 10 MBq. In some embodiments, the radioactivity is measured at the timepoint immediately prior to use of the dendrimer.

### Combinations

Drugs are often administered in combination with other drugs, especially during chemotherapy. Accordingly, in some embodiments the dendrimer is administered in combination with one or more further pharmaceutically active agents, for example one or more further anti-cancer agents/drugs. The dendrimer and the one or more further pharmaceutically active agents may be administered simultaneously, subsequently or separately. For example, they may be administered as part of the same composition, or by administration of separate compositions.

The one or more further pharmaceutically active agents may for example be anti-cancer agents for therapy of prostate cancers, brain cancers, breast cancers, testicular cancers, ovarian cancers, stomach cancers, adenocarcinomas of the lung, gastric cancers, pancreatic cancers, salivary duct carcinomas, oesophageal cancers, or uterine cancers (e.g., uterine serious endometrial carcinoma).

The one or more further pharmaceutically active agents may for example be anti-cancer agents for therapy of colorectal cancer, stomach cancer, pancreas cancer, prostate cancer or breast cancer.

Examples of further pharmaceutically active agents include chemotherapeutic and cytotoxic agents, small molecule cytotoxics, tyrosine kinase inhibitors, checkpoint inhibitors, EGFR inhibitors, antibody therapies, taxanes (e.g. paclitaxel, docetaxel, cabazitaxel, nab-paclitaxel), topoisomerase inhibitors (e.g. SN-38, irinotecan (CPT-11), topotecan, silatecan, cositecan, exatecan, lurtotecan, gimatecan, belotecan, or rubitecan), nucleoside analogues, and aromatase inhibitors.

Still further examples of pharmaceutically active agents which may be used in combination with the dendrimer include radiosensitisers, pharmaceutically active agents which reduce DNA repair, immunotherapy agents, survival signalling inhibitors and antihypoxics.

In some embodiments the pharmaceutically active agent is a radio sensitiser. In some embodiments the pharmaceutically active agent reduces DNA repair. In some embodiments the pharmaceutically active agent is selected from the group consisting of an agent targeting; DNA-dependent protein kinase; checkpoint kinase 1; poly(ADP-ribose) polymerase such as olaparib; ataxia telangiectasia and/or Rad3-related protein such as AZD6738. In some embodiments the pharmaceutically active agent is an immunotherapy agent. In some embodiments the immunotherapy agent is selected from the group consisting of agents which block co-inhibitory molecules; CTLA-4, cytotoxic T-lymphocyte-associated protein 4; PD-1, programmed cell death protein 1; checkpoint inhibitors. In some embodiments the pharmaceutically active agent is a survival signalling inhibitor (proapoptotic). In some embodiments the agent is selected from the group consisting of an agent targeting: mTOR, mechanistic target of rapamycin; PI3K, phosphoinositide 3-kinase; and NF-κB, nuclear factor-kappa-B; In some embodiments the pharmaceutically active agent is an antihypoxic. In some embodiments the agent is selected from the group consisting of an agent targeting: CA9, carbonic anhydrase 9, HIF-1-α, hypoxia-inducible factor 1-alpha, and UPR, unfolded protein response. In some embodiments the agent is tirapazamine.

### Dendrimer Preparation

Radioactive materials are hazardous substances, and handling steps using such materials are ideally minimised. It is desirable to introduce the radionuclide component into the dendrimers only at a late stage, ideally at a time just prior to use of the conjugates.

The dendrimers comprising a radionuclide as described herein may be prepared from an intermediate and a radionuclide. The intermediate dendrimer may contain at least some terminal groups that comprise a complexing group for complexing a radionuclide.

Accordingly, there is provided an intermediate for producing a radionuclide-containing dendrimer which comprises:

i) a core unit (C); and

ii) building units (BU);

wherein the core unit is covalently attached to at least two building units;

the dendrimer having from two to six generations of building units; wherein building units of different generations are covalently attached to one another; and

the dendrimer further comprising:

iii) one or more first terminal groups attached to an outermost building unit, wherein each first terminal group comprising a complexation group for complexing a radionuclide; and

iv) one or more second terminal groups attached to an outermost building unit, wherein each second terminal group comprising a pharmacokinetic-modifying moiety.

It will be appreciated that any one or more various embodiments or examples as described herein for the core unit (C), building unit (BU), terminal groups, or dendrimer, may also be provided for the intermediate dendrimer.

In another embodiment, there is provided a kit for producing a dendrimer according to any aspects, embodiments or examples thereof as described herein, the kit comprising an intermediate dendrimer and a radionuclide, each independently provided according to any aspects, embodiments or examples thereof as described herein.

A process for producing a dendrimer according to at least some embodiments or examples as described herein may comprise contacting the intermediate dendrimer with the radionuclide to produce the dendrimer. Any suitable means of producing the dendrimer may be used. For example, intermediate and a radionuclide salt may be admixed in an aqueous solvent containing an appropriate buffer so that complexation of the radionuclide occurs.

The above described kit and processes can be used to provide an effective in-clinic preparation of pharmaceutical compositions by radiolabelling the dendrimers in the clinic before administration.

The intermediate dendrimer may itself be produced, for example, from a precursor dendrimer provided with a functional group, either as part of an outermost building unit or as part of a first terminal group attached to an outermost building unit, for reaction with and introduction of a complexation group. Alternatively, the precursor dendrimer may be in protected form, having a protecting group that can be deprotected and then reacted to introduce a complexation group and thus prepare an intermediate dendrimer.

For example, a complexing group may be reacted with the precursor dendrimer to form an intermediate dendrimer comprising at least some terminal groups comprising a complexation group for complexing a radionuclide.

A precursor dendrimer may for example comprise:

i) a core unit (C); and

ii) building units (BU);

wherein the core unit is covalently attached to at least two building units;

the dendrimer having from two to six generations of building units; wherein building units of different generations are covalently attached to one another; and

the dendrimer further comprising:

iii) one or more first terminal groups attached to an outermost building unit, the first terminal group comprising a functional group available for reaction to introduce a complexation group, or comprising a protected version of such a functional group; and

iv) one or more second terminal groups attached to an outermost building unit, wherein each second terminal group comprises a pharmacokinetic-modifying moiety.

Alternatively, a precursor dendrimer may comprise:

i) a core unit (C); and

ii) building units (BU);

wherein the core unit is covalently attached to at least two building units;

the dendrimer having from two to six generations of building units; wherein building units of different generations are covalently attached to one another; and

the dendrimer further comprising:

iii) outermost building units comprising a functional group available for reaction to introduce a complexation group, or comprising a protected version of such a functional group; and

iv) one or more second terminal groups attached to an outermost building unit, wherein each second terminal group comprises a pharmacokinetic-modifying moiety.

Examples of suitable functional groups available for reaction to introduce a complexation group include amine functional groups present on an outermost lysine building unit. Suitable protecting groups may include, for example, Boc or Cbz protecting groups.

A process for producing a dendrimer according to at least some embodiments or examples as described herein may comprise optionally deprotecting any protecting groups if present on the precursor dendrimer, contacting the precursor dendrimer with a complexation group to produce an intermediate dendrimer, and contacting the intermediate dendrimer with the radionuclide to produce the dendrimer.

Third terminal groups may be provided on the intermediate dendrimer by further reaction with a residue of a pharmaceutically active agent. It will be appreciated that the complexation group, radionuclide, third terminal groups, residue of a pharmaceutically active agent, and pharmaceutically active agent, may be each independently provided according to any embodiments or examples thereof as described herein.

It may also be desirable to introduce the pharmaceutically active agent at a late stage of the process, for example given that that component is often a valuable component of the dendrimer.

Accordingly, in some embodiments, a precursor dendrimer comprising:

i) a core unit (C); and

ii) building units (BU);

wherein the core unit is covalently attached to at least two building units;

the dendrimer having from two to six generations of building units; wherein building units of different generations are covalently attached to one another; and

the dendrimer further comprising:

iii) outermost building units comprising functional groups available for reaction (e.g. amino groups); and

iv) one or more second terminal groups attached to an outermost building unit, wherein each second terminal group comprises a pharmacokinetic-modifying moiety;

may be reacted with a moiety comprising a complexation group, such that some of the available sites on the outermost building units contain a complexation group. Subsequently, other available functional groups on the outermost building units may for example be reacted with a linker-pharmaceutically active agent group, such that other available sites contain a pharmaceutically active agent, thereby producing an intermediate dendrimer. The intermediate dendrimer may then be reacted with a radionuclide (e.g. radionuclide salt) such that the radionuclide is complexed, producing the final dendrimer.

By way of example the reactions of functional groups with a moiety containing a complexation groups, and with linker-pharmaceutically active agent groups, may involve amide formation reactions, e.g. between amino groups present on the outermost building unit, and carboxylic acid or activated carboxyl groups (e.g. active esters) present on the other partner.

In such a process, the proportion of sites on the surface of the final dendrimer which contain a first terminal group versus a third terminal group may be controlled by, for example controlling the stoichiometry of the reagents used in the reactions.

As discussed above, the number of first, second and, where present, third terminal groups which form part of the dendrimer can be varied so as to tailor the properties of the dendrimer as desired. In some embodiments, the intermediate dendrimer (i.e. the dendrimeric material prior to complexation of radionuclide) has a molar ratio of complexation group to pharmaceutically active agent in the range of from 1:1 to 1:100, or from 1:1 to 1:50, or from 1:1 to 1:40, or from 1:1 to 1:30, or from 1:1 to 1:20, or from 1:1 to 1:10, or from 1:2 to 1:100, or from 1:2 to 1:50, or from 1:2 to 1:40, or from 1:2 to 1:30, or from 1:2 to 1:20, or from 1:2 to 1:10, or from 1:5 to 1:100, or from 1:5 to 1:50, or from 1:5 to 1:40, or from 1:5 to 1:40, or from 1:5 to 1:30, or from 1:5 to 1:20, or from 1:5 to 1:10, or from 1:10 to 1:100, or from 1:10 to 1:50, or from 1:10 to 1:40, or from 1:10 to 1:30, or from 1:10 to 1:20.

Precursor dendrimers comprising a core, building units (e.g. lysine building units) and second terminal groups comprising pharmacokinetic modifying groups such as PEG groups, are described in, for example WO2007/082331 and WO2012/167309.

The above processes may comprise various embodiments or examples of the precursor dendrimer, intermediate dendrimer, and dendrimer, as described herein.

There is also provided a kit for producing a dendrimer according to any aspects, embodiments or examples thereof as described herein, the kit comprising a precursor dendrimer, a complexation group, and a radionuclide, each independently provided according to any aspects, embodiments or examples thereof as described herein.

The kit may provide a sufficient amount of radionuclide to administer a suitable dose of radioactivity to the subject, and will typically also contain a suitable quantity of precursor dendrimer to complex that amount of radionuclide. In some embodiments, the kit comprises radionuclide which provides an amount of radioactivity in the range of up to 50 GBq, from 1 to 20 GBq, or from 1 to 10 GBq. In some embodiments, the kit comprises radionuclide which provides an amount of radioactivity in the range of from 0.1 to 10 MBq, from 0.1 to 5 MBq, from 0.1 to 2 MBq, from 0.1 to 1 MBq, from 0.5 to 10 MBq, from 1 to 10 MBq, from 1 to 5 MBq, from 5 to 10 MBq, or about 1, about 2, about 3, about 4, about 5, about 6, about 7, about 8, about 9 or about 10 MBq. In some embodiments, the radioactivity is measured at the timepoint immediately prior to complexation of the radionuclide by the dendrimer, i.e. immediately prior to use.

### EXAMPLES

### Core Unit and Building Unit Synthesis

BHALys[Lys]₃₂[α-NH₂TFA]₃₂[ε-PEGx]₃₂, in which X refers to the approximate molecular weight of the PEG groups, was produced by synthetic methods analogous to those described in WO2007/082331.

The terminology BHALys[Lys]₃₂ refers to a dendrimer having a BHALys core unit, and five generations of lysine building units such that it contains 32 lysine building units at the outermost layer i.e.: BHALys [Lys]₂ [Lys]₄ [Lys]₈ [Lys]₁₆ [Lys]₃₂.

### Example 1

(a) BHALys[Lys]₃₂[(α-NH₂)₃₀(α-DFO)₂(ε-PEG₂₀₀₀)₃₂]
(b) BHALys[Lys]₃₂[(α-TDA-DTX)₃₀(α-DFO)₂(ε-PEG₂₀₀₀)₃₂]

To a stirred solution of BHALys[Lys]₃₂[(α-NH₂.TFA)(ε-PEG₂₀₀₀)₃₂] (151 mg, 1.98 μmol) (prepared in an analogous manner to that described in Example 1) in DMF (4.0 mL) was added p-SCN-deferoxamine (p-SCN-DFO) (4.83 mg, 6.41 μmol, 3.24 eq) followed by addition of NMM (56 μL, 514 μmol). The resulting reaction mixture was stirred at ambient temperature for 3.5 h, half (2.0 mL) of the reaction mixture was removed and stirred in a separate vial (Reaction A). To the remaining solution (Reaction B) was added a solution of TDA-DTX (thiodiacetic acid-docetaxel) (60 mg, 58.9 μmol) and PyBOP (35 mg, 67.8 μmol) in DMF (1.5 mL), followed by further addition of NN (56 μL, 514 μmol). Both reaction mixtures were then left to stir at ambient temperature overnight.

### Reaction A (Control):

After 19 h, the reaction mixture was concentrated in vacuo to dryness then dissolved in MeOH (1.0 mL) and purified by SEC. The product-containing fractions were combined and concentrated in vacuo, and the resulting residue dissolved in MQ water, filtered (0.45 μm acrodisc filter) and lyophilised to give compound 1a as a white flocculent solid (65.8 mg).

HPLC (hydrophilic, ammonium formate) Rt=8.98 min. ¹H NMR (300 MHz, CD₃OD-d₄) δ (ppm): 1.29-2.06 (m, 468H), 2.43-2.53 (m, 13H), 2.71-2.82 (m, 13H), 3.06-3.28 (m, 121H), 3.36 (s, 96H), 3.39-3.42 (m, 39H), 3.51-4.06 (m, 5781H), 4.25-4.45 (m, 36H), 6.17 (broad s, 1H), 7.24-7.58 (m, 19H), 8.09 (s, 1H). ¹H NMR analysis suggests approx. 2.3 DFO/dendrimer; % (w/w) of DFO=2.3%.

### Reaction B (TDA-DTX):

After 24 h, the reaction mixture was concentrated in vacuo to dryness then dissolved in MeOH (1.0 mL) and purified by SEC. The product-containing fractions were combined and concentrated in vacuo, and the resulting residue dissolved in MQ water, filtered (0.45 μm acrodisc filter) and lyophilised to give compound 1b as a white flocculent solid (96.8 mg). HPLC (hydrophilic, ammonium formate) Rt=8.51 min. ¹H NMR (300 MHz, CD₃OD-d₄) δ (ppm): 0.80-2.66 (m, 1183H), 3.36 (s, 96H), 3.38-3.41 (m, 47H), 3.50-3.77 (m, 5100H), 3.85-3.90 (m, 62H), 3.98 (broad s, 67H), 4.12-4.48 (m, 129H), 4.96-5.07 (m, 41H), 5.19-5.49 (m, 80H), 5.54-5.75 (m, 31H), 6.00-6.26 (m, 26H), 7.16-7.97 (m, 255H), 8.05-8.22 (m, 62H). ¹H NMR analysis suggests approx. 30 DTX/dendrimer and 2.3 DFO/dendrimer; % (w/w) of DFO=1.7%.

### Example 2

BHALys[Lys]₃₂[(α-TDA)₃₁(α-DFO)₁(ε-PEG₂₀₀₀)₃₂]

To a stirred solution of BHALys[Lys]₃₂[(α-NH₂.TFA)(ε-PEG₂₀₀₀)₃₂] (100 mg, 1.32 mol) and p-SCN-deferoxamine (1.0 mg, 1.32 μmol, 1.0 equiv.) in DMF (2.5 mL) was added NMM (10 μL, 91 μmol). The reaction mixture was stirred at ambient temperature for 5 h after which time TDA (11 mg, 84.4 μmol) was added and the contents stirred overnight. The reaction mixture was concentrated in vacuo then dissolved in MeOH (1.0 mL) and purified by SEC. The product-containing fractions were combined and concentrated in vacuo, and the resulting residue dissolved in MQ water, filtered (0.45 μm acrodisc filter) and lyophilised to give the title product as a fluffy white powder (89.4 mg). HPLC (hydrophilic, ammonium formate) Rt=8.43 min. ¹H NMR (300 MHz, CD₃OD-d₄) δ (ppm): 1.29-1.99 (m, 371H), 3.19-3.26 (m, 77H), 3.36 (s, 96H), 3.38-3.49 (m, 149H), 3.50-3.77 (m, 5131), 3.84-3.90 (m, 35H), 4.01 (broad s, 59H), 4.27-4.43 (m, 74H), 6.19 (broad s, 1H), 7.26-7.36 (m, 10H), 8.09 (s, 1H). ¹H NMR analysis suggests approx. 1.0 DFO/dendrimer; % (w/w) of DFO=1.0%.

### Example 3

BHALys[Lys]₃₂[(α-DGA-CTX)₃₁(α-DFO)₁(ε-PEG₂₀₀₀)₃₂]

To a stirred solution of BHALys[Lys]₃₂[(α-NH₂.TFA)(ε-PEG₂₀₀₀)₃₂] (71.2 mg, 0.93 μmol) in DMF (1.0 mL) was added p-SCN-deferoxamine (1.0 mg, 1.33 μmol, 1.42 equiv.), followed by addition of NMM (20 μL, 182 μmol). The resulting cloudy reaction mixture was stirred at ambient temperature for 3 h, after which time a solution of DGA-CTX (diglycolic acid-cabazitaxel) (56.1 mg, 58.9 μmol) and PyBOP (29.8 mg, 57.3 μmol) in DMF (2.0 mL) was added followed by further addition of NMM (20 μL, 182 μmol). After 19 h, the reaction mixture was concentrated in vacuo then dissolved in MeOH (1.0 mL) and purified by SEC. The product-containing fractions were combined and concentrated in vacuo, and the resulting residue dissolved in MQ water, filtered (0.45 μm acrodisc filter) and lyophilised to give the title product as a white flocculent solid (84.6 mg). HPLC (hydrophilic, ammonium formate) Rt=9.19 min. ¹H NMR (300 MHz, CD₃OD-d₄) δ (ppm): 0.88-2.51 (m, 1283H), 2.65-2.80 (m, 55H), 3.36 (s, 96H), 3.37-3.41 (m, 103H), 3.50-4.57 (m, 5045H), 4.97-5.07 (m, 33H), 5.30-5.46 (m, 52H), 5.54-5.69 (m, 29H), 6.08-6.24 (m, 30H), 7.23-7.73 (m, 248H), 8.05-8.17 (m, 59H). ¹H NMR analysis suggests approx. 31 CTX/dendrimer and 1.0 DFO/dendrimer; % (w/w) of DFO=0.74%.

### Example 4

(a) BHALys[Lys]₃₂[(α-NH₂)₃₀(α-DOTA)₂(ε-PEG₂₀₀₀)₃₂]
(b) BHALys[Lys]₃₂[(α-DGA-CTX)₂₇(α-DOTA)₂(ε-PEG₂₀₀₀)₃₂]

To a stirred solution of BHALys[Lys]₃₂[(α-NH₂.TFA)(ε-PEG₂₀₀₀)₃₂] (301 mg, 3.97 μmol) in DMF (6.0 mL) was added p-SCN-Bn-DOTA (8.13 mg, 11.8 μmol, 2.98 eq), followed by addition of NMM (114 μL, 1.03 mmol). The resulting reaction mixture was stirred at ambient temperature for 4.5 h, then a portion (2.0 mL) of the solution was removed to a separate vial (Reaction A). To the remaining solution (Reaction B), was added a solution of TDA-CTX (105 mg, 110.4 μmol) and PyBOP (57.0 mg, 109.5 μmol) in DMF (2 mL). After 45 min NN (56 μL, 514 μmol) was added and both reaction mixtures were then left to stir at ambient temperature overnight.

### Reaction A (Control):

After 24 h, the reaction mixture was concentrated in vacuo to dryness, then dissolved in MeOH (1.0 mL) and purified by SEC. The product-containing fractions were combined and concentrated in vacuo, and the resulting residue dissolved in MQ water, filtered (0.45 μm acrodisc filter) and lyophilised to give compound 4a as a white solid (82.5 mg). ¹H NMR (300 MHz, CD₃OD-d₄) δ (ppm): 1.17-2.29 (m, 401H), 3.36 (s, 96H), 3.39-3.43 (m, 43H), 3.50-4.08 (m, 5564H), 4.21-4.67 (m, 84H), 6.17 (broad s, 1H), 7.18-7.64 (m, 18H), 8.09 (s, 1H). ¹H NMR analysis suggests approx. 2.1 DOTA/dendrimer; % (w/w) of DOTA=2.0%.

### Reaction B (TDA-CTX):

After 19 h, the reaction mixture was concentrated in vacuo to dryness, dissolved in MeOH (1.0 mL) and purified by SEC. The product-containing fractions were combined and concentrated in vacuo, and the resulting residue dissolved in MQ water, filtered (0.45 μm acrodisc filter) and lyophilised to give compound 4b as a white solid (238 mg). LCMS (hydrophilic, TFA) Rt=8.83 min. ¹H NMR (300 MHz, CD₃OD-d4) δ (ppm): 0.95-2.76 (m, 1020H), 3.36 (s, 96H), 3.38-3.41 (m, 83H), 3.52-4.56 (m, 5081H), 4.99-5.11 (m, 34H), 5.38-5.61 (m, 74H), 6.16 (broad s, 26H), 7.29-8.17 (m, 300H). ¹H NMR analysis suggests approx. 26.5 CTX/dendrimer and 2.1 DOTA/dendrimer; % (w/w) of DOTA=1.5%.

### Example 5

BHALys[Lys]₃₂[(α-NHAc)₃₀(α-DOTA)₂(ε-PEG₂₀₀₀)₃₂]

To a stirred solution of BHALys[Lys]32[(α-DOTA)2(α-NH2.TFA)30(ε-PEG2000)32](63 mg, 860 μmol) in DMF (0.6 mL) was added TEA (25 μL, 228 μmol), followed by acetic anhydride (41 μL, 430 μmol). The ensuing reaction mixture was stirred at ambient temperature overnight. The reaction mixture was concentrated in vacuo, dissolved in MeOH (1.0 mL) and purified by SEC. The product-containing fractions were combined and concentrated in vacuo, and the resulting residue dissolved in MQ water, filtered (0.45 μm acrodisc filter) and lyophilised to give compound 4c as a white solid (52.2 mg). The solid was dissolved in MQ water (50 mL) and purified by ultrafiltration (minimate) in water. After collection of 11 DV of permeate, the retentate was concentrated, filtered (0.22 μm acrodisc filter) then lyophilised to give the title compound (52.2 mg). HPLC (hydrophilic, ammonium formate) Rt=8.56 min. ¹H NMR (300 MHz, D2O) δ (ppm): 1.16-1.90 (m, 359H), 2.02 (broad s, 101H), 3.01-3.31 (m, 133H), 3.38 (s, 96H), 3.45-3.48 (m, 43H), 3.52-4.40 (m, 5267H), 6.09 (broad s, 1H), 7.13-7.54 (m, 17H). ¹H NMR analysis suggests approx. 1.8 DOTA/dendrimer; % (w/w) of DOTA=1.7%.

### Example 6

BHALys[Lys]₃₂[α-DGA-C20-SN38]₂₈[α-DFO]₂[ε-PEG₂₀₀₀]₃₂

To a stirred solution of BHALys[Lys]₃₂[α-NH₂.TFA]₃₂[F-PEG₂₀₀₀]₃₂ (455 mg, 6.00 mol) and NMM (169 μL, 1.54 mmol) in DMF (3 mL) was added p-SCN-Bn-Deferoxamine (13.7 mg, 18.2 μmol). The suspension was left to stir at ambient temperature under a nitrogen atmosphere for 4 h 40 min. After this time, a portion of the hazy reaction mixture (1.75 mL) was added to a stirred solution of DGA-C20-SN-38 (82.4 mg, 162 μmol) and PyBOP (85.2 mg, 164 μmol) in DMF (0.5 mL). The resulting mixture was diluted with DMF (1.0 mL) and stirred overnight under a nitrogen atmosphere. After 16 hours the volatiles were removed in vacuo, the residue dissolved in MeOH (1.0 mL) and filtered (0.7 μm acrodisc filter, followed by 0.45 μm and 0.2 m acrodisc filters) before purification by SEC. The product-containing fractions were combined and concentrated in vacuo, and the resulting residue dissolved in MQ water, filtered (0.45 μm acrodisc filter) and lyophilised to give the title compound as a yellow solid (243 mg). HPLC (hydrophilic, ammonium formate) Rt=8.61 min. ¹H NMR (300 MHz, CD₃OD-d4) δ (ppm): 0.32-2.53 (m, 622H), 2.53-3.26 (m, 182H), 3.36 (s, 97H), 3.37-4.04 (m, 5,530H), 4.04-4.73 (m, 145H), 4.92-6.42 (m, 68H), 6.81-8.19 (m, 123H). ¹H NMR analysis suggests approx. 27.5 SN-38/dendrimer and 1.7 DFO/dendrimer; % (w/w) of DFO=1.5%.

### Example 7

(a) BHALys[Lys]₃₂[α-NH_{2]30}[α-DOTA]₂[ε-PEG₂₀₀₀]₃₂
(b) BHALys[Lys]₃₂[α-DGA-C20-SN38]₃₀[α-DOTA]₂[ε-PEG₂₀₀₀]₃₂

To a stirred solution of BHALys[Lys]₃₂[α-NH₂.TFA]₃₂[F-PEG₂₀₀₀]₃₂ (456 mg, 6.00 mol) and NMM (169 μL, 1.54 mmol) in DMF (9 mL) was added p-SCN-Bn-DOTA (12.6 mg, 18.3 μmol). The mixture was left to stir at ambient temperature under a nitrogen atmosphere for 3.5 h, then a portion (3.75 mL) of the reaction mixture was removed to a separate vial (Reaction A). The remaining solution was added to a stirred solution of DGA-C20-SN-38 (82.7 mg, 163 μmol) and PyBOP (85.3 mg, 164 μmol) in DMF (1.75 mL) (Reaction B). Both reaction mixtures were stirred overnight.

### Reaction A:

After 16 hours the reaction mixture was concentrated in vacuo to dryness, then dissolved in MeOH (1.0 mL) and purified by SEC. The product-containing fractions were combined and concentrated in vacuo, and the resulting residue dissolved in MQ water, filtered (0.45 μm acrodisc filter) and lyophilised to give compound 7a as an off-white solid (175 mg). HPLC (hydrophilic, ammonium formate) Rt=8.58 min. ¹H NMR (300 MHz, CD₃OD-d4) δ (ppm): 0.78-2.41 (m, 388H), 2.64-3.29 (m, 122H), 3.36 (s, 95H), 3.38-4.19 (m, 5,546H), 4.19-4.59 (m, 37H), 6.98-7.82 (m, 18H). ¹H NMR analysis suggests approx. 2.4 DOTA/dendrimer; % (w/w) of DOTA=2.2%.

### Reaction B:

After 16 hours the reaction mixture was concentrated in vacuo to dryness, then dissolved in MeOH (1.0 mL) and purified by SEC. The product-containing fractions were combined and concentrated in vacuo, and the resulting residue dissolved in MQ water, filtered (0.45 μm acrodisc filter) and lyophilised to give compound 7b as a yellow solid (269 mg). HPLC (hydrophilic, ammonium formate) Rt=8.59 min. ¹H NMR (300 MHz, CD₃OD-d4) δ (ppm): 0.28-2.51 (m, 580H), 2.53-3.25 (m, 178H), 3.36 (s, 98H), 3.37-4.06 (m, 5,546H), 4.07-4.69 (m, 128H), 4.91-6.10 (m, 66H), 6.71-8.26 (m, 167H). ¹H NMR analysis suggests approx. 35.3 SN-38/dendrimer and 2.4 DOTA/dendrimer; % (w/w) of DOTA=1.8%.

### Example 8

(a) BHALys[Lys]₃₂[(α-NOTA)₂(α-NH₂)₃₀(ε-PEG₁₁₀₀)₃₂]
(b) BHALys[Lys]₃₂[(α-NOTA)₂(α-NHAc)₃₀(ε-PEG₁₁₀₀)₃₂]

To a stirred solution of BHALys[Lys]₃₂[(α-NH₂.TFA)(ε-PEG₂₀₀₀)₃₂] (60 mg, 807 nmol) and p-SCN-Bn-NOTA (1.0 mg, 1.61 μmol, 2.0 eq) in DMF (0.5 mL) was added NMM (10 μL, 91.0 μmol). The resulting reaction mixture was stirred at ambient temperature for 5 h, then half (0.25 mL) of the reaction mixture was removed and concentrated in vacuo (Reaction A). The remaining solution (Reaction B) was treated with acetic anhydride (24 μL, 258 μmol) and left to stir overnight.

### Reaction A:

The crude material was taken up in MQ water (5.0 mL) then divided evenly across two PD-10 desalting columns. The collected filtrate was combined and lyophilised to give compound 8a as a fluffy white powder (28.1 mg). HPLC (hydrophilic, TFA) Rt=8.18 min. ¹H NMR (300 MHz, CD₃OD-d₄) δ (ppm): 1.17-2.04 (m, 392H), 3.12-3.28 (m, 97H), 3.36 (s, 96H), 3.39-3.42 (m, 39H), 3.51-3.80 (m, 5584H), 3.86-3.89 (m, 35H), 3.97-4.06 (m, 60H), 4.22-4.47 (m, 34H), 6.18 (broad s, 1H), 7.20-7.60 (m, 20H), 8.08 (s, 1H). ¹H NMR analysis suggests approx. 2.5 NOTA/dendrimer; % (w/w) of NOTA=1.9%.

### Reaction B:

After 17 h, the reaction mixture was concentrated in vacuo then taken up in MQ water (5.0 mL) and divided evenly across two PD-10 desalting columns. The collected filtrate was combined and lyophilised to give compound 8b as a fluffy white powder (32.5 mg). HPLC (hydrophilic, TFA) Rt=8.32 min. ¹H NMR (300 MHz, CD₃OD-d₄) δ (ppm): 1.17-1.89 (m, 372H), 2.00 (broad s, 97H), 3.18-3.29 (m, 86H), 3.36 (s, 96H), 3.38-3.42 (m, 38H), 3.51-3.77 (m, 5535H), 3.84-3.90 (m, 37H), 3.97-4.07 (m, 62H), 4.20-4.49 (m, 62H), 6.17 (broad s, 1H), 7.16-7.61 (m, 18H), 8.07 (broad s, 1H). ¹H NMR analysis suggests approx. 2.0 NOTA/dendrimer; % (w/w) of NOTA=1.5%.

### Example 9

BHALys[Lys]₃₂[(α-NOTA)₃(α-TDA-CTX)₂₈(ε-PEG₂₀₀₀)₃₂]

To a stirred solution of BHALys[Lys]₃₂[(α-NH₂.TFA)(ε-PEG₂₀₀₀)₃₂] (51 mg, 686 nmol) and p-SCN-Bn-NOTA (1.3 mg, 2.32 μmol, 3.4 eq) in DMF (0.5 mL) was added NMM (14 μL, 132 μmol). The resulting reaction mixture was stirred at ambient temperature for 4 h, after which time a solution of TDA-CTX (43 mg, 43.9 μmol) and PyBOP (23 mg, 43.9 μmol) in DMF (1.0 mL) was added. The ensuing reaction mixture was left to stir overnight then concentrated in vacuo. The contents were then dissolved in MeOH (1.0 mL) and purified by SEC. The product-containing fractions were combined and concentrated in vacuo, and the resulting residue dissolved in MQ water, filtered (0.45 μm acrodisc filter) and lyophilised to give the title compound as a fluffy white powder (61.0 mg). HPLC (hydrophilic, TFA) Rt=8.87 min. ¹H NMR (300 MHz, CD₃OD-d₄) δ (ppm): 0.90-2.43 (m, 877H), 2.64-3.19 (m, 151H), 3.36 (s, 96H), 3.38-3.41 (m, 84H), 3.50-4.59 (m, 4808H), 4.96-5.13 (m, 29H), 5.31-5.61 (m, 64H), 6.16 (broad s, 24H), 7.29-8.13 (m, 296H). ¹H NMR analysis suggests approx. 28 CTX/dendrimer and 3.0 NOTA/dendrimer; % (w/w) of NOTA=1.7%.

### Example 10

(a) BHALys[Lys]₃₂[(α-NOTA)₂(α-NH₂)₃₀(ε-PEG₅₇₀)₃₂]
(b) BHALys[Lys]₃₂[(α-NOTA)₂(α-NHAc)₃₀(ε-PEG₅₇₀)₃₂]

To a stirred solution of BHALys[Lys(α-NH₂.TFA)(ε-PEG₅70)]₃₂ (60 mg, 1.99 μmol) and p-SCN-Bn-NOTA (2.2 mg, 3.98 μmol, 2.0 eq) in DMF (0.5 mL) was added NMM (10 μL, 91.0 μmol). The resulting reaction mixture was stirred at ambient temperature overnight. After this time, half (0.25 mL) of the reaction mixture was removed and concentrated in vacuo (Reaction A). The remaining solution (Reaction B) was treated with acetic anhydride (60 μL, 636 μmol) and left to stir overnight.

### Reaction A:

The crude material was taken up in MQ water (5.0 mL) then divided evenly across two PD-10 desalting columns. The collected filtrate was combined and lyophilised to give compound 10a as a pale yellow sticky solid (22.4 mg). HPLC (hydrophilic, TFA) Rt=7.51 min. ¹H NMR (300 MHz, CD₃OD-d₄) δ (ppm): 1.29-2.04 (m, 431H), 2.41-2.52 (m, 89H), 3.13-3.26 (m, 119H), 3.36 (s, 96H), 3.39-3.44 (m, 24H), 3.52-4.50 (m, 1651H), 6.18 (broad s, 1H), 7.18-7.63 (m, 19H). ¹H NMR analysis suggests approx. 2.3 NOTA/dendrimer; % (w/w) of NOTA=4.6%.

### Reaction B:

The reaction mixture was concentrated in vacuo then taken up in MQ water (5.0 mL) and divided evenly across two PD-10 desalting columns. The collected filtrate was combined and lyophilised to give compound 10b as a pale yellow sticky solid (26.9 mg). HPLC (hydrophilic, TFA) Rt=8.10 min. ¹H NMR (300 MHz, CD₃OD-d₄) δ (ppm): 1.29-2.05 (m, 546H), 2.40-2.52 (m, 85H), 3.12-3.26 (m, 137H), 3.36 (s, 96H), 3.39-3.44 (m, 22H), 3.53-4.00 (m, 1621H), 4.16-4.47 (m, 103H), 6.18 (broad s, 1H), 7.22-7.56 (m, 21H), 7.82-8.14 (m, 27H). ¹H NMR analysis suggests approx. 2.3 NOTA/dendrimer; % (w/w) of NOTA=4.4%.

### Example 11

BHALys[Lys]₃₂[(α-CHX-A-DTPA)₁₀(ε-PEG₂₀₀₀)₃₂]

A mixture of BHALys[Lys(α-NH₂.TFA)(ε-PEG₂₀₀₀)]₃₂ (25 mg, 332 nmol) and CHX-A-DTPA (9.7 mg, 13.8 μmol, 41.5 eq) in ammonium formate buffer (100 mM, pH 9, 1.0 mL) was stirred overnight at ambient temperature. The reaction mixture was then diluted with MQ water (1.5 mL) and passed through a PD-10 desalting column. The collected filtrate was combined and lyophilised to give the title compound as a white solid (32.5 mg). HPLC (hydrophilic, ammonium formate) Rt=8.53 min. ¹H NMR (300 MHz, D₂O) δ (ppm): 0.92-2.50 (m, 420H), 3.01-3.34 (m, 136H), 3.40 (s, 96H), 3.54-4.45 (m, 4906H), 6.10 (broad s, 1H), 7.15-7.82 (m, 51H). ¹H NMR analysis suggests approx. 10 DTPA/dendrimer; % (w/w) of DTPA=7.7%.

### Example 12

BHALys[Lys]₃₂[(α-CHX-A-DTPA)₃(α-TDA-DTX)₂₆(ε-PEG₂₀₀₀)₃₂]

To a stirred solution of BHALys[Lys(α-NH₂.TFA)(ε-PEG₂₀₀₀)]₃₂ (109 mg, 1.45 μmol) in DMF (2.0 mL) was added DIPEA (33 μL, 189 μmol). After 5-10 min, CHX-A-DTPA (3 mg, 4.26 μmol, 2.9 eq) was added and the ensuing reaction mixture stirred at ambient temperature for 1 h. After this time, the reaction mixture was then added to a stirred solution of TDA-DTX (67 mg, 70.8 μmol), PyBOP (31 mg, 60.2 μmol) in DMF (1.0 mL) and the contents stirred overnight. The ensuing reaction mixture was left to stir overnight then concentrated in vacuo. The crude material was dissolved in MeOH (1.0 mL) and purified by SEC. The product-containing fractions were combined and concentrated in vacuo, and the resulting residue dissolved in MQ water, filtered (0.45 μm acrodisc filter) and lyophilised to give the title compound as a white solid (123 mg). HPLC (hydrophilic, ammonium formate) Rt=6.51 min. ¹H NMR (300 MHz, CD₃OD-d₄) δ (ppm): 0.87-2.55 (m, 1380H), 3.06-3.25 (m, 89H), 3.36 (s, 96H), 3.39-3.42 (m, 49H), 3.51-4.05 (m, 4965H), 5.31-5.64 (m, 120H), 6.03-6.23 (m, 34H), 7.26-7.67 (m, 224H), 8.06-8.18 (m, 57H). ¹H NMR analysis suggests approx. 26 DTX/dendrimer and 3.0 CHX-A-DTPA/dendrimer; % (w/w) of CHX-A-DTPA=1.8%.

### Example 13

BHALys[Lys]₃₂[(α-CHX-A-DTPA)₂(α-NH₂)₃₀(ε-PEG₂₆₀)₃₂]

### Reaction A:

A mixture of BHALys[Lys(α-NH₂.TFA)(ε-PEG₂₆₀₀)]₃₂ (50 mg, 532 nmol) and CHX-A-DTPA (1.0 mg, 1.45 μmol, 2.7 eq) in ammonium formate buffer (100 mM, pH 9, 1.0 mL) was stirred at ambient temperature for 1 h. The reaction mixture was then diluted with MQ water to 5 mL then divided evenly across two PD-10 desalting columns. The collected filtrate was combined and lyophilised to give the title compound as a white solid (47.2 mg). HPLC (hydrophilic, ammonium formate) Rt=6.0 min. ¹H NMR (300 MHz, CD₃OD-d4) δ (ppm): 1.04-2.11 (m, 381H), 3.12-3.28 (m, 79H), 3.36 (s, 96H), 3.38-3.42 (m, 57H), 3.47-4.46 (m, 6823H), 6.17 (broad s, 1H), 7.24-7.64 (m, 21H). ¹H NMR analysis suggests approx. 2.7 CHX-A-DTPA/dendrimer; % (w/w) of CHX-A-DTPA=1.7%.

### Reaction B:

To a stirred solution of BHALys[Lys(α-NH₂.TFA)(ε-PEG₂₆₀₀)]₃₂ (50 mg, 532 nmol) in DMF (0.5 mL) was added DIPEA (13 μL, 74.6 μmol). After 5-10 min, CHX-A-DTPA (1.0 mg, 1.45 μmol, 2.7 eq) was added and the ensuing reaction mixture stirred at ambient temperature for 1 h. The reaction mixture was concentrated in vacuo, diluted with MQ water (5 mL), then divided evenly across two PD-10 desalting columns. The collected filtrate was combined and lyophilised to give the title compound as a white solid (41.9 mg). HPLC (hydrophilic, ammonium formate) Rt=6.0 min. ¹H NMR (300 MHz, CD₃OD-d₄) δ (ppm): 1.02-2.25 (m, 369H), 3.12-3.28 (m, 63H), 3.36 (s, 96H), 3.38-3.42 (m, 53H), 3.51-4.50 (m, 6736H), 6.17 (broad s, 1H), 7.22-7.57 (m, 18H). ¹H NMR analysis suggests approx. 2.0 CHX-A-DTPA/dendrimer; % (w/w) of CHX-A-DTPA=1.3%.

### Example 14

BHALys[Lys]₃₂[(α-CHX-A-DTPA)₂(α-TDA-DTX)₂₁(ε-PEG₂₆₀₀)₃₂]

To a stirred solution of BHALys[Lys]₃₂[(α-CHX-A-DTPA)₂(α-NH₂)₃₀(ε-PEG₂₆₀₀)]₃₂ (69 mg, 723 nmol) in DMF (2.0 mL) was added DIPEA (15 μL, 86.1 μmol). After 5 min, the reaction mixture was added to a stirred solution of TDA-DTX (31 mg, 33.0 μmol), PyBOP (16 mg, 30.7 μmol) in DMF (1.0 mL) and the contents stirred at ambient temperature overnight. The reaction mixture was concentrated in vacuo, dissolved in MeOH (1.0 mL) and purified by SEC. The product-containing fractions were combined, concentrated in vacuo, and the resulting residue dissolved in MQ water, filtered (0.45 μm acrodisc filter) and lyophilised to give the title compound as a white solid (60.4 mg). ¹H NMR (300 MHz, CD₃OD-d₄) δ (ppm): 0.89-2.65 (m, 1074H), 3.05-3.25 (m, 88H), 3.36 (s, 96H), 3.39-3.42 (m, 55H), 3.52-3.81 (m, 6451H), 3.86-3.90 (m, 60H), 3.94-4.06 (m, 56H), 5.29-5.78 (m, 90H), 5.99-6.30 (m, 20H), 7.26-7.70 (m, 188H), 8.11-8.13 (m, 45H). ¹H NMR analysis suggests approx. 21 DTX/dendrimer and 2.0 CHX-A-DTPA/dendrimer; % (w/w) of CHX-A-DTPA=1.1%.

### Example 15

BHALys[Lys]₃₂[(α-CHX-A-DTPA)₂(α-NH₂)₃₀(ε-PEG₂₀₀₀)₃₂]

A mixture of BHALys[Lys(α-NH₂.TFA)(ε-PEG₂₀₀₀)]₃₂ (109 mg, 1.45 μmol) and CHX-A-DTPA (2.0 mg, 2.90 μmol, 2.0 eq) in ammonium formate buffer (100 mM, pH 9, 2.0 mL) was stirred at ambient temperature for 1 h. The reaction mixture was then diluted with MQ water to 10 mL then divided evenly across four PD-10 desalting columns. The collected filtrate was combined and lyophilised to give the title compound as a white solid (112 mg). HPLC (hydrophilic, ammonium formate) Rt=8.55 min. ¹H NMR (300 MHz, CD₃OD-d4) δ (ppm): 1.15-2.12 (m, 419H), 3.17-3.28 (m, 84H), 3.36 (s, 96H), 3.38-3.42 (m, 43H), 3.47-3.80 (m, 5460H), 3.84-3.89 (m, 46H), 4.00-4.07 (m, 67H), 4.24-4.48 (m, 35H), 6.18 (broad s, 1H), 7.21-7.51 (m, 20H), 8.07 (broad s, 2H). ¹H NMR analysis suggests approx. 2.5 CHX-A-DTPA/dendrimer; % (w/w) of CHX-A-DTPA=2.0%.

### Example 16

BHALys[Lys]₃₂[(α-DTPA)₂(α-NH₂)₃₀(ε-PEG₂₆₀₀)₃₂]

To a stirred solution of BHALys[Lys(α-NH₂.TFA)(ε-PEG₂₆₀₀)]₃₂ (50 mg, 532 nmol) in DMF (0.5 mL) was added DIPEA (13 μL, 74.6 μmol). After 5-10 min, p-SCN-Bn-DTPA (1.0 mg, 1.54 μmol, 2.9 eq) was added and the ensuing reaction mixture stirred at ambient temperature for 30 min. The reaction mixture was concentrated in vacuo, diluted with MQ water (5 mL), then divided evenly across two PD-10 desalting columns. The collected filtrate was combined and lyophilised to give the title compound as a white solid (27.0 mg). ¹H NMR (300 MHz, CD₃OD-d₄) δ (ppm): 1.08-2.23 (m, 351H), 3.17-3.28 (m, 72H), 3.36 (s, 96H), 3.38-3.42 (m, 55H), 3.50-3.80 (m, 7032H), 3.85-3.89 (m, 58H), 3.96-4.05 (m, 68H), 4.23-4.52 (m, 35H), 6.19 (broad s, 1H), 7.20-7.57 (m, 16H). ¹H NMR analysis suggests approx. 1.5 DTPA/dendrimer; % (w/w) of DTPA=1.1%.

### Example 17

BHALys[Lys]₃₂[(α-DTPA)₂(α-TDA-DTX)₂₆(ε-PEG₂₆₀)₃₂]

To a stirred solution of BHALys[Lys]₃₂[(α-DTPA)₂(α-NH₂)₃₀(ε-PEG₂₆₀₀)]₃₂ (15.3 mg, 161 nmol) in DMF (0.5 mL) was added DIPEA (4 μL, 20.6 μmol). After 5 min, the reaction mixture was added to a stirred solution of TDA-DTX (1.3 mg, 1.38 μmol), PyBOP (3.5 mg, 6.69 μmol) in DMF (1.0 mL) and the contents stirred at ambient temperature overnight. The reaction mixture was concentrated in vacuo, dissolved in MeOH (1.0 mL) and purified by SEC. The product-containing fractions were combined, concentrated in vacuo, and the resulting residue dissolved in MQ water, filtered (0.45 μm acrodisc filter) and lyophilised to give the title compound as a fluffy white solid (8.3 mg). ¹H NMR (300 MHz, CD₃OD-d₄) δ (ppm): 0.99-2.53 (m, 770H), 3.13-3.26 (m, 50H), 3.36 (s, 96H), 3.38-3.41 (m, 56H), 3.47-3.77 (m, 6440H), 3.84-3.88 (m, 72H), 3.95-4.07 (m, 65H), 4.13-4.49 (m, 102H), 5.22-5.47 (m, 50H), 5.57-5.70 (m, 22H), 6.06-6.21 (m, 19H), 7.27-8.15 (m, 280H). ¹H NMR analysis suggests approx. 26 DTX/dendrimer and 1.5 DTPA/dendrimer; % (w/w) of DTPA=0.85%.

### Example 18

BHA-[Lys]₈[(α-(MeTzPh-PEG₄-PEG₂₄)₁(α-NH₂)₇(ε-NHPEG₁₁₀₀)₈], G3, Compound 18

A stirred solution of BHA[Lys(NH₂.TFA)(NHPEG₁₁₀₀)]₈ (100 mg, 0.00786 mmol, 1.0 eq) in DMF (300 μL) was prepared at RT. To this was added MeTzPh-PEG₄-PEG₂₄-CO₂H (16 mg, 0.01 mmol, 1.3 eq), PyBOP (8 mg, 0.013 mmol, 1.6 eq) and DMF (200 μL). The reaction mixture was stirred for 3 min before addition of NMM (40 mg, 50 μL, 0.38 mmol, 48 eq). The contents were protected from light and stirred overnight at RT. The reaction mixture was diluted with MQ water and lyophilized overnight. The lyophilized material was taken up in MeOH (1 mL) and purified by SEC (400 drops/tube, MeOH sephadex LH20, 35 drops/min). The product-containing fractions were checked by HPLC and collected in 2 different fractions. Each fraction was concentrated under reduced pressure, then the resulting residue taken up in MQ water, filtered (0.45 μm acrodisc filter) and freeze dried to yield the title product as a pink solid (69 mg, 66%).

HPLC (C8 XBridge, 3×100 mm) gradient: 5% ACN/H₂O (0-1 min), 5-80% ACN (1-7 min), 80% ACN (7-12 min), 80-5% ACN (12-13 min), 5% ACN (13-15 min), 214 nm, 0.4 mL/min, Rf (min)=8.4 (broad peak). ¹HNMR (300 MHz, D₂O) δ (ppm): 1.00-2.00 (m, 90H), 2.51 (t, 3H), 2.60 (br s, 3H), 3.00-3.12 (m, 6H), 3.12-3.35 (br s, 27H), 3.35-3.45 (m, 26H), 3.45-4.15 (m, 937H), 4.15-4.45 (m, 12H), 6.12 (s, 1H), 7.15-7.50 (m, 12H), 8.40-8.50 (m, 2H).

### Example 19

BHA-[Lys]₈[(α-MeTzPh-PEG₄PEG₂₄)₁(α-DFO)₂(Glu-VC-PAB-MMAE)₅(ε-NHPEG₁₁₀₀)₈], Compound 19

A stirred solution of p-SCN-Deferoxamine (2.0 mg, 2.66 μmol) in DMSO (100 μL) was prepared at RT. To this was added BHA-[Lys]₈[(α-(MeTzPh-PEG₄-PEG₂₄)₁(α-NH₂)₇(ε-NHPEG₁₁₀₀)₈] (compound 18) (17.0 mg, 1.27 μmol) in DMF (200 μL). The ensuing reaction mixture was stirred for 3 min before addition of NN (10 μL, 91.0 μmol). The resulting solution was protected from light and stirred for 4 h at RT. PyBOP (7.0 mg, 13.5 μmol) was added and after 5 min the reaction mixture was added to neat HO-Glu-VC-PAB-MMAE (9.17 mg, 7.41 μmol). The ensuing reaction mixture was left to stand overnight. The reaction mixture was diluted with PBS buffer (4.5 mL) and divided across 4 Amicon Ultra centrifugal filters (10K MWCO) and the filters centrifuged (14K rcf, 15 min). The retentate was diafiltered against PBS (400 μL, 14K rcf, 15 min×10 times). The retentate was combined to give a pink coloured solution, approximate concentration of 16 mg in 2 mL. HPLC (C8 XBridge, 3×100 mm) gradient: 5% ACN/H₂O (0-1 min), 5-80% ACN (1-7 min), 80% ACN (7-12 min), 80-5% ACN (12-13 min), 5% ACN (13-15 min), 214 nm, 0.4 mL/min, Rt (min)=9.3-9.7 min (broad peak).

### Example 20

MeTzPh-PEG₄PEG₂₄-CO[N(PN)₂][Lys]₈[(α-DFO)₂(α-Glu-VC-PAB-MMAE)₆(ε-NHPEG₁₁₀₀)₈], Compound 20

A stirred solution of p-SCN-Deferoxamine (2.1 mg, 2.79 μmol) in DMSO (100 μL) was prepared at RT. To this was added MeTzPh-PEG₄PEG₂₄-CO[N(PN)₂][Lys(α-NH₂.HCl)(8-NHPEG₁₁₀₀)]₈ (as described in WO 2008/017125) (17.0 mg, 1.35 μmol) in DMF (200 μL). The ensuing reaction mixture was stirred for 3 min before addition of NMM (10 μL, 91.0 μmol). The resulting solution was protected from light and stirred for 4 h at RT. PyBOP (7.0 mg, 13.5 μmol) was added and after 5 min the reaction mixture was added to neat HO-Glu-VC-PAB-MMAE (9.76 mg, 7.89 μmol). The ensuing reaction mixture was left to stand overnight. The reaction mixture was diluted with PBS buffer (4.5 mL) and divided across 4 Amicon Ultra centrifugal filters (10K MWCO) and the filters centrifuged (14K rcf, 15 min). The retentate was diafiltered against PBS (400 μL, 14K rcf, 15 min×10 times). The retentate was combined to give a pink coloured solution, approximate concentration of 16 mg in 2 mL. HPLC (C8 XBridge, 3×100 mm) gradient: 5% ACN/H₂O (0-1 min), 5-80% ACN (1-7 min), 80% ACN (7-12 min), 80-5% ACN (12-13 min), 5% ACN (13-15 min), 214 nm, 0.4 mL/min, Rt (min)=8.7-9.8 min (broad peak).

### Purification Techniques for Dendrimers Prior to Incorporation of Radionuclide

Size Exclusion Chromatography (SEC) was performed using Sephadex LH-20 (column height=370 mm, diameter=25 mm), eluent=MeOH gravity elution, drip rate ˜1 drop per second, 400 drops per fraction. Product-containing fractions stained positive with BaCl₂/I₂ stain.

HPLC (hydrophilic, ammonium formate) method: XBridge C8 (3.5 μm, 3×100 mm) column. Samples were eluted at a flow rate of 0.4 mL/min (buffer 100 mM ammonium formate) as follows: 5 to 80% ACN/water (1-7 min); 80% ACN/water (7-12 min); 80 to 5% ACN/water (12-13 min); 5% ACN/water (13-15 min).

LCMS (hydrophilic, TFA) method: XBridge C18 (3.5 μm, 3×100 mm) column. Samples were eluted at a flow rate of 0.4 mL/min (buffer 0.1% TFA) as follows: 20 to 90% ACN/water (1-10 min); 90% ACN/water (10-11 min); 90 to 20% ACN/water (11-12 min); 20% ACN/water (12-15 min).

General Procedure for complexing Gd³⁺

To a stirred solution of the dendrimer (30 mg) in pH 5.5 ammonium acetate buffer (500 μL) was added a solution of 0.1M GdCl₃ (pH 7, 50 equivalents of Gd³⁺). The reaction mixture was stirred at room temperature for 16 h and then concentrated to a volume of ˜100 μL by centrifugation at room temperature (6.5 min at 14 k rcf) using Amicon Ultra spin columns (MWCO=10 kDa). The concentrate was diluted with water (400 μL) and again concentrated to a volume of 100 μL by centrifugation. This procedure was repeated with water (×2), 50 mM DTPA (×2) and water (×3). The retentate was then transferred to a vial and lyophilized to give the desired product.

List of Synthesized Dendrimer Conjugates

| Compound | MW | Complexation | Radioactiv | | |
|---|---|---|---|---|---|
| # | (kDa) | Group | e metal | Drug | Compound Details |
| 1a | 74.3 | DFO | | N/A | BHALys[Lys]₃₂[(α- |
| | | | | | NH₂)₃₀(α-DFO)₂(ε- |
| | | | | | PEG₂₀₀₀)₃₂] |
| 1b | 101.6 | DFO | ⁸⁹Zr | DTX | BHALys[Lys]₃₂[(α-TDA- |
| | | | | | DTX)₃₀(α-DFO)₂(ε- |
| | | | | | PEG₂₀₀₀)₃₂] |
| 2 | 76.4 | DFO | | N/A | BHALys[Lys]₃₂[(α- |
| | | | | | TDA)₃₁(α-DFO)₁(ε- |
| | | | | | PEG₂₀₀₀)₃₂] |
| 3 | 102.2 | DFO | ⁸⁹Zr | CTX | BHALys[Lys]₃₂[(α-DGA- |
| | | | | | CTX)₃₁(α-DFO)₁(ε- |
| | | | | | PEG₂₀₀₀)₃₂] |
| 4a | 73.5 | DOTA | | N/A | BHALys[Lys]₃₂[(α- |
| | | | | | NH₂)₃₀(α-DOTA)₂(ε- |
| | | | | | PEG₂₀₀₀)₃₂] |
| 4b | 98.3 | DOTA | ¹⁷⁷Lu, Gd³⁺ | CTX | BHALys[Lys]₃₂[(α-DGA- |
| | | | | | CTX)₂₇(α-DOTA)₂(ε- |
| | | | | | PEG₂₀₀₀)₃₂] |
| 5 | 74.7 | DOTA | ¹⁷⁷Lu, Gd³⁺ | N/A | BHALys[Lys]₃₂[(α- |
| | | | | | NHAc)₃₀(α-DOTA)₂(ε- |
| | | | | | PEG₂₀₀₀)₃₂] |
| 6 | 86.9 | DFO | | SN38 | BHALys[Lys]₃₂[α-DGA- |
| | | | | | C20—SN38]₂₈[α-DFO]₂[ε- |
| | | | | | PEG₂₀₀₀]₃₂ |
| 7a | 73.8 | DOTA | | N/A | BHALys[Lys]₃₂[α- |
| | | | | | NH₂]₃₀[α-DOTA]₂[ε- |
| | | | | | PEG₂₀₀₀]₃₂ |
| 7b | 91.1 | DOTA | Gd³⁺ | SN38 | BHALys[Lys]₃₂[α-DGA- |
| | | | | | C20—SN38]₃₀[α- |
| | | | | | DOTA]₂[ε-PEG₂₀₀₀]₃₂ |
| 8a | 72.1 | NOTA | | N/A | [BHALys][Lys]₃₂[(α- |
| | | | | | NOTA)₂(α-NH₂)₃₀(ε- |
| | | | | | PEG₁₁₀₀)₃₂] |
| 8b | 73.0 | NOTA | | N/A | [BHALys][Lys]₃₂[(α- |
| | | | | | NOTA)₂(α-NHAc)₃₀(ε- |
| | | | | | PEG₁₁₀₀)₃₂] |
| 9 | 98.9 | NOTA | | CTX | [BHALys][Lys]₃₂[(α- |
| | | | | | NOTA)₃(α-TDA- |
| | | | | | CTX)₂₈(ε-PEG₂₀₀₀)₃₂] |
| 10a | 27.8 | NOTA | | N/A | [BHALys][Lys]₃₂[(α- |
| | | | | | NOTA)₂(α-NH₂)₃₀(ε- |
| | | | | | PEG₅₇₀)₃₂] |
| 10b | 29.1 | NOTA | | N/A | [BHALys][Lys]₃₂[(α- |
| | | | | | NOTA)₂(α-NHAc)₃₀(ε- |
| | | | | | PEG₅₇₀)₃₂] |
| 11 | 77.6 | CHX-A-DTPA | | N/A | [BHALys][Lys]₃₂[(α- |
| | | | | | CHX-A-DTPA)₁₀(ε- |
| | | | | | PEG₂₀₀₀)₃₂] |
| 12 | 97.4 | CHX-A-DTPA | | DTX | BHALys[Lys]₃₂[(α-CHX- |
| | | | | | A-DTPA)₃(α-TDA- |
| | | | | | DTX)₂₆(ε-PEG₂₀₀₀)₃₂] |
| 13 | 92.0 | CHX-A-DTPA | | N/A | BHALys[Lys]₃₂[(α-CHX- |
| | | | | | A-DTPA)₂(α-NH₂)₃₀(ε- |
| | | | | | PEG₂₆₀₀)₃₂] |
| 14 | 110.9 | CHX-A-DTPA | | DTX | [BHALys][Lys]₃₂[(α- |
| | | | | | (CHX-A-DTPA)₂(α- |
| | | | | | TDA-DTX)₂₁(ε- |
| | | | | | PEG₂₆₀₀)₃₂] |
| 15 | 73.2 | CHX-A-DTPA | | N/A | BHALys[Lys]₃₂[(α-CHX- |
| | | | | | A-DTPA)₂(α-NH₂)₃₀(ε- |
| | | | | | PEG₂₀₀₀)₃₂] |
| 16 | 91.3 | DTPA | | N/A | [BHALys][Lys]₃₂[(α- |
| | | | | | DTPA)₂(α-NH₂)₃₀(ε- |
| | | | | | PEG₂₆₀₀)₃₂] |
| 17 | 115.3 | DTPA | | DTX | [BHALys][Lys]₃₂[(α- |
| | | | | | DTPA)₂(α-TDA-DTX)₂₆ |
| | | | | | (ε-PEG₂₆₀₀)₃₂] |
| 18 | | | | N/a | BHA-[Lys]₈[(α- |
| | | | | | (MeTzPh-PEG₄- |
| | | | | | PEG₂₄)₁(α-NH₂)₇(ε- |
| | | | | | NHPEG₁₁₀₀)₈] |
| 19 | | DFO | | MMAE | BHA-[Lys]₈ [(α- |
| | | | | | MeTzPh- |
| | | | | | PEG₄PEG₂₄)₁(α- |
| | | | | | DFO)₂(α-Glu-VC- |
| | | | | | PAB-MMAE)₅ (ε- |
| | | | | | NHPEG₁₁₀₀)₈] |
| 20 | | DFO | | MMAE | MeTzPh-PEG₄PEG₂₄- |
| | | | | | CO[N(PN)₂][Lys]₈ [(α- |
| | | | | | DFO)₂(α-Glu-VC- |
| | | | | | PAB-MMAE)₆(ε- |
| | | | | | NHPEG₁₁₀₀)₈] |

General Procedure for Radiolabeling with Cu-64 and RadioTLC Analysis of Dendrimers

To a solution of a NOTA-containing dendrimer sample in 0.1M ammonium acetate buffer (pH 5.5), was added a solution of a solution of ⁶⁴Cu(OAc)₂ (50 μL, 70 MBq) and sample was stirred at room temperature for 1 h. Samples were then buffer exchanged into phosphate-buffered saline using Zeba Spin Desalting Columns (7 kDa MWCO, Thermo Fisher Scientific). An aliquot of the reaction mixture was removed, added to a large excess of EDTA (1000:1 molar excess) and incubated for 15 min. 1 μL samples of each solution were taken and spotted on thin layer chromatography paper (Agilent iTLC-SG Glass microfiber chromatography paper impregnated with silica gel) and run with 50 mM diethylenetriaminepentaacetic acid (DTPA) as the eluent. Control experiments were conducted to monitor the elution behaviour of unbound ⁶⁴Cu for quality control. Plates were then imaged on a Bruker In Vivo MS FX Pro imaging system using a radioisotopic phosphor screen. Samples with radiochemical purity (RCP) greater than 95% were used for imaging experiments.

### Example 19

Tumour Imaging Study with Radionuclide-Containing Dendrimers—Prostate Cancer

The accumulation of two different dendrimer constructs in two different murine xenograft models of prostate cancer (DU145 and PC3 cell lines) was investigated. The two different constructs were compound 1b and 3 which are pre-conjugated with DFO, which were labelled with 89Zr for subsequent imaging studies. The biodistribution was measured by PET-CT out to 9 days in two different tumour xenografts and then validated by ex vivo gamma scintillation of excised organs at day 9.

Dendrimers were labelled and purified, validated by radioTLC prior to injection into the animals. Imaging was conducted in a cohort of n=4 mice for each cell line and each dendrimer. Standard health of the mice was monitored by score sheet and mouse weight over the complete timeframe of the study.

Radiolabeling with Zr-89 and RadioTLC Analysis of Dendrimers

91 μL of Zr-89 oxalate in 1 M oxalic acid (Perkin Elmer) was diluted with 78 μL 1 M Na₂CO₃ to neutralise pH. Dendrimers 1b and 3 were prepared in 0.5 M HEPES (pH 7.5). 33 μL neutralised ⁸⁹Zr stock (approx. 15 MBq) was added to aliquots of each dendrimer (146 μg) to give 100-fold excess of the dendrimer to Zr-89 and labelling was allowed to proceed at ambient temperature for 1 h. Samples were then buffer exchanged into phosphate-buffered saline using Zeba Spin Desalting Columns (7 kDa MWCO, Thermo Fisher Scientific). 1 μL samples of each solution were taken and spotted on thin layer chromatography paper (Agilent iTLC-SG Glass microfiber chromatography paper impregnated with silica gel) and run with 50 mM diethylenetriaminepentaacetic acid (DTPA) as the eluent. Control experiments were conducted to monitor the elution behaviour of unbound Zr-89 for quality control. Plates were then imaged on a Bruker In Vivo MA FX Pro imaging system using a radioisotopic phosphor screen.

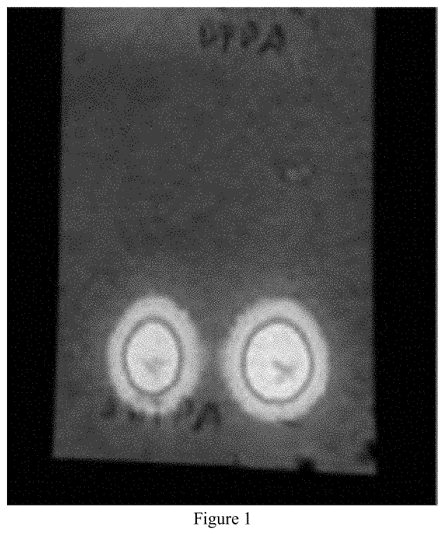

After incubation at 500-fold excess of dendrimer, dendrimer 1b was allowed to label for 1 h and then washed with 1000-fold excess of DTPA. After spin purification, a maximum purity of approx. 90% was achieved (TLC shown in FIG. 1).

### Animals

Healthy male Balb/C nude mice (˜20 g) from 8 weeks old were obtained from the ARC and used for this study. Mice were imported into the animal holding facility and monitored for 1 week prior to the study in order to acclimatise to the environment prior to injection of cells. All animals were provided with free access to food and water before and during the imaging experiments which were approved by the Animal Ethics Committee.

### Tumour Initiation and Growth

All mice were acquired at 8 weeks of age but were injected at slightly different times to give comparable tumours at the time of imaging. This was based on previous experience with these models and growth rates.

5×10⁶ DU-145 cells (in 50 μL saline) were injected (27 G needle) into the left flank of 9 week old male balb/c nude mice. Tumours were allowed to grow for 4 weeks prior to injection of imaging compounds.

1×10⁶ PC3 cells (in 50 μL saline) were injected (27 G needle) into the left flank of 11 week old male balb/c nude mice. Tumours were allowed to grow for 2 weeks prior to injection of imaging compounds. All tumours were palpable at the time of imaging, with sizes ˜3-5 mm at the time of the imaging experiment.

### Study Details

The following table describes the injection details for all mice used in the study.

| | | Injection | Injected |
|---|---|---|---|
| | | Volume | Dose |
| Mouse ID | Compound | (μL) | (MBq) |
| M566 | DU145-Compound 1b | 100 | 2.35 |
| M567 | DU145-Compound 1b | 100 | 2.54 |
| M568 | DU145-Compound 1b | 100 | 2.28 |
| M569 | DU145-Compound 1b | 100 | 1.51 |
| M570 | PC3-Compound 1b | 100 | 2.52 |
| M571 | PC3-Compound 1b | 100 | 2.56 |
| M572 | PC3-Compound 1b | 100 | 2.58 |
| M573 | PC3-Compound 1b | 100 | 2.58 |
| M574 | DU145-Compound 3 | 100 | 2.81 |
| M575 | DU145- Compound 3 | 100 | 2.96 |
| M576 | DU145- Compound 3 | 100 | 2.95 |
| M577 | DU145- Compound 3 | 100 | 2.94 |
| M578 | PC3-Compound 3 | 100 | 3.33 |
| M579 | PC3- Compound 3 | 100 | 3.09 |
| M580 | PC3-Compound 3 | 100 | 2.97 |
| M581 | PC3- Compound 3 | 100 | 2.88 |

### Results

Under optimised conditions, two dendrimers (compounds 1b and 3) were labelled with Zr-89 and used for biodistribution analyses. Apart from the obvious growth of the tumour lesion, no adverse health effects were recorded for any of the animals during this study.

### PET-CT Imaging

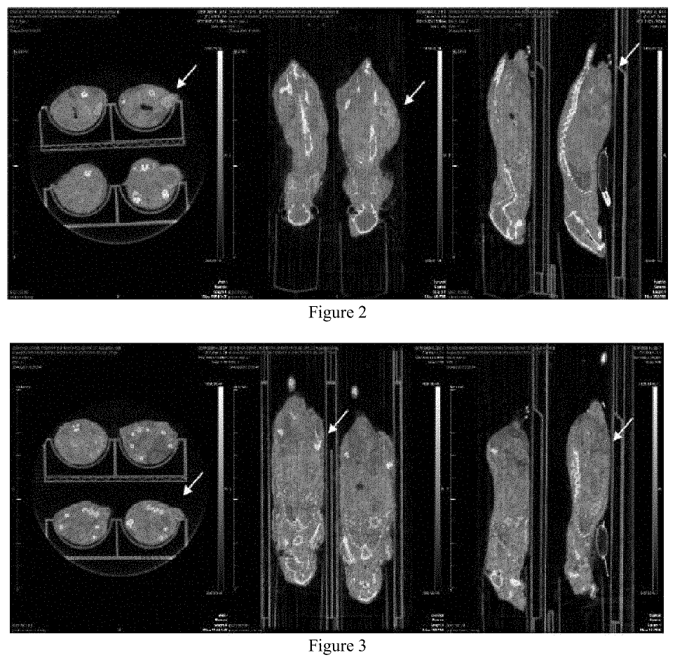

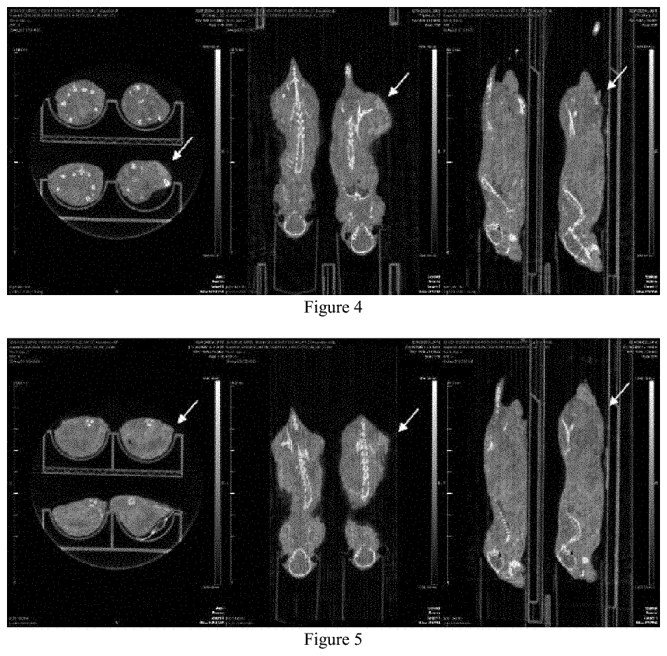

Mice (n=4 per group) bearing DU-145 or PC3 xenografts were injected with Zr-89 labelled dendrimers (compounds 1b and 3) via tail-vein. Images were taken at 8 hrs, 24 hrs, 48 hrs, 72 hrs, 144 hrs and 216 hrs post-injection. At 216 hrs post-injection, the organs were removed and signal intensity quantified by ex vivo gamma analysis. Faecal pellets were also measured for activity at this timepoint. FIGS. 2 and 3 show representative images of compound 1b for the DU-145 and PC3 xenografts, respectively, 6 days post-injection of the dendrimer. FIGS. 4 and 5 show representative images of compound 3 for the DU-145 and PC3 xenografts, respectively, 6 days post-injection of the dendrimer.

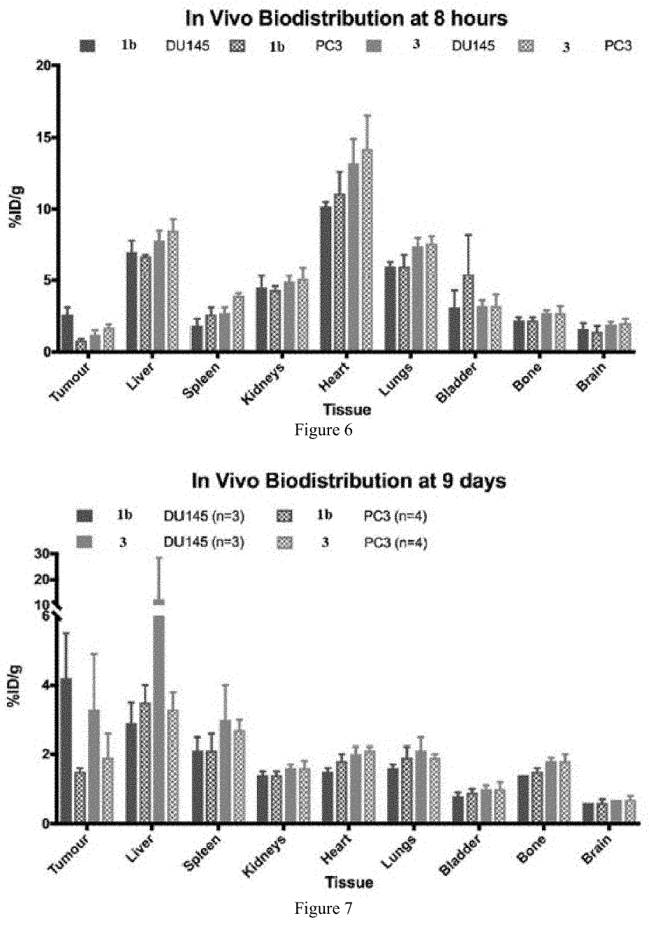

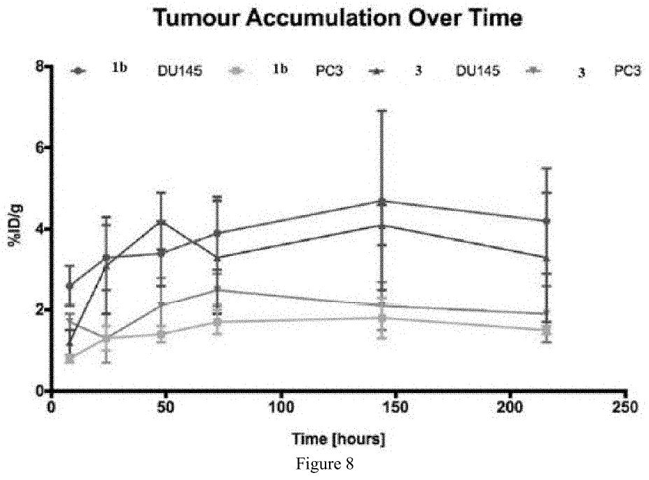

In order to better understand the biodistribution profiles of the different cohorts, accumulation plots for the organs as determined in vivo and ex vivo are provided (see FIGS. 6-7 and 19). To further evaluate trends in the data, comparisons between the tumour uptake at different time points was plotted to show the temporal effect of accumulation as a function of tumour type. This data was extracted from the in vivo images by drawing a region of interest around the tumour mass at the different timepoints (FIG. 8).

### Conclusion

All mice showed good tumour growth and tumour accumulation was shown to reach approximately 4% ID/g for the DU-145 tumours and 2% ID/g for the PC3 tumours. There was no observable difference between the two different dendrimers. The difference accumulation is likely due to level of vasculature and heterogeneity between tumour types, however this would require further investigation including tissue analysis.

In terms of rate of accumulation, FIG. 8 shows that all dendrimers show slow accumulation up to 6 days at which time maximum uptake is observed. This is indicative of an EPR mechanism contributing to the accumulation owing to long circulation of the dendrimers.

No unusual accumulation in clearance organs was observed, with the liver and spleen signal showing expected concentration ranges as typically observed for similar systems. The presence of significant signal in the faeces of all animal cohorts at 9 days suggests that the dendrimer is still being cleared through this route. Likewise, in vivo images show that there is statistically significant signal intensity in the bladder at 9 days for all animals, suggesting probable clearance of metabolic products through renal mechanisms. The signal intensity measured in the bone sample at 9 days post-injection were around or just slightly higher than background, suggesting that there was minimal accumulation in this tissue.

### Example 20

### Tumour Imaging Study With Radionuclide-containing Dendrimers—Pancreatic and Breast Cancer

The accumulation of two different dendrimer constructs in two different murine xenograft models of pancreatic and breast cancer (PANC-1 and MDA-MB-468 cell lines, respectively) was investigated. The two different constructs were compounds 1b and 3 which were already pre-conjugated with DFO, and ready for labelling with 89Zr for subsequent imaging studies. The biodistribution was measured by PET-CT out to 9 days in two different tumour xenografts and then validated by ex vivo gamma scintillation of excised organs at day 9.

Dendrimers were labelled and purified, validated by radioTLC prior to injection into the animals. Both dendrimers labelled well and were purified to high purity suitable for imaging with a single purification step. Standard health of the mice was monitored by score sheet and mouse weight over the complete timeframe of the study.

Radiolabeling with Zr-89 and RadioTLC Analysis of Dendrimers

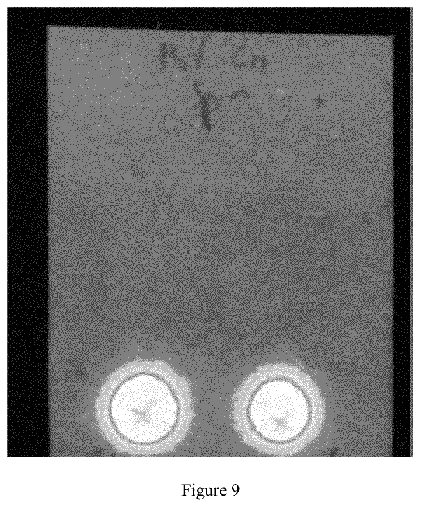

91 μL 89Zr oxalate in 1 M oxalic acid (Perkin Elmer) was diluted with 78 μL 1 M Na2CO3 to neutralize pH. Dendrimers 1b and 3 were prepared in 0.5 M HEPES (pH 7.5). 33 L neutralized 89Zr stock (approximately 15 MBq) was added to aliquots of each dendrimer (146 μg) to give 100-fold excess of the dendrimer to 89Zr and labelling was allowed to proceed at room temperature for 1 hour. Samples were then buffer exchanged into phosphate-buffered saline using Zeba Spin Desalting Columns (7 kDa MWCO, Thermo Fisher Scientific). 1 μL samples of each solution were taken and spotted on thin layer chromatography paper (Agilent iTLC-SG Glass microfiber chromatography paper impregnated with silica gel) and run with 50 mM diethylenetriaminepentaacetic acid (DTPA) as the eluent. Control experiments were conducted to monitor the elution behaviour of unbound 89Zr for quality control. Plates were then imaged on a Bruker In Vivo MS FX Pro imaging system using a radioisotopic phosphor screen (TLC shown in FIG. 9).

### Animals

Healthy female Balb/C nude mice (˜20 g) from 8 weeks old were obtained from the ARC and used for this study. Mice were imported into the animal holding facility and monitored for 1 week prior to the study in order to acclimatise to the environment prior to injection of cells. All animals were provided with free access to food and water before and during the imaging experiments which were approved by the Animal Ethics Committee

### Tumour Initiation and Growth

All mice were acquired at 8 weeks of age, but were injected at slightly different times to give comparable tumours at the time of imaging. This was based on previous experience with these models and growth rates.

5×10⁶ PANC-1 cells (in 50 uL saline) were injected (27 G needle) into the left flank of 9 week old male balb/c nude mice. Tumours were allowed to grow for 4 weeks prior to injection of imaging compounds.

5×10⁶ MDA-MB-468 cells (in 50 uL saline) were injected (27 G needle) into the left flank of 11 week old male balb/c nude mice. Tumours were allowed to grow for 2 weeks prior to injection of imaging compounds.

All tumours were palpable at the time of imaging, with sizes ˜3-5 mm at the time of the imaging experiment. It should be noted that these tumours had vastly different growth rates (MDA-MB-468 more aggressive in growth than PANC-1), and this can lead to observable differences in images at longer time-points (c.f. % ID/g). The PANC-1 tumours were very slow to grow and had a much lower take-rate than MDA-MB-468.

### Study Details

The following table describes the injection details for all mice used in the study.

| | | Injection | Injected |
|---|---|---|---|
| | | Volume | Dose |
| Mouse ID | Compound | (μL) | (MBq) |
| F656 | MDA-MB-468- | 100 | 2.87 |
| | Compound 1b | | |
| F657 | MDA-MB-468- | 100 | 2.84 |
| | Compound 1b | | |
| F658 | MDA-MB-468- | 100 | 2.75 |
| | Compound 1b | | |
| F659 | MDA-MB-468- | 100 | 2.89 |
| | Compound 1b | | |
| F660 | MDA-MB-468- | 100 | 3.05 |
| | Compound 3 | | |
| F661 | MDA-MB-468- | 100 | 3.01 |
| | Compound 3 | | |
| F662 | MDA-MB-468- | 100 | 3.02 |
| | Compound 3 | | |
| F663 | MDA-MB-468- | 100 | 3.21 |
| | Compound 3 | | |
| F664 | PANC-1- | 100 | 2.77 |
| | Compound 1b | | |
| F665 | PANC-1- | 100 | 2.68 |
| | Compound 1b | | |
| F666 | PANC-1- | 100 | 2.78 |
| | Compound 1b | | |
| F667 | PANC-1- | 100 | No tumour |
| | Compound 1b | | |
| F668 | PANC-1- | 100 | 2.84 |
| | Compound 3 | | |
| F669 | PANC-1- | 100 | 3.03 |
| | Compound 3 | | |
| F670 | PANC-1- | 100 | No tumour |
| | Compound 3 | | |
| F671 | PANC-1- | 100 | No tumour |
| | Compound 3 | | |

### Results

Under optimised conditions, the two dendrimers were labelled with 89Zr and used for biodistribution analyses. Apart from the obvious growth of the tumour lesion, no adverse health effects were recorded for any of the animals during this study.

### PET-CT Imaging

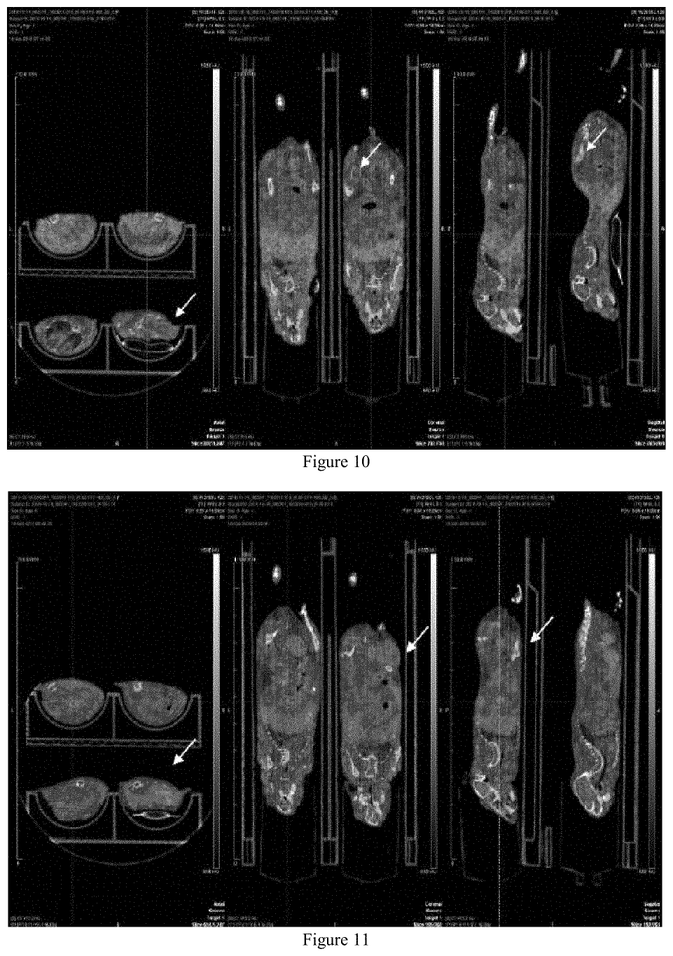

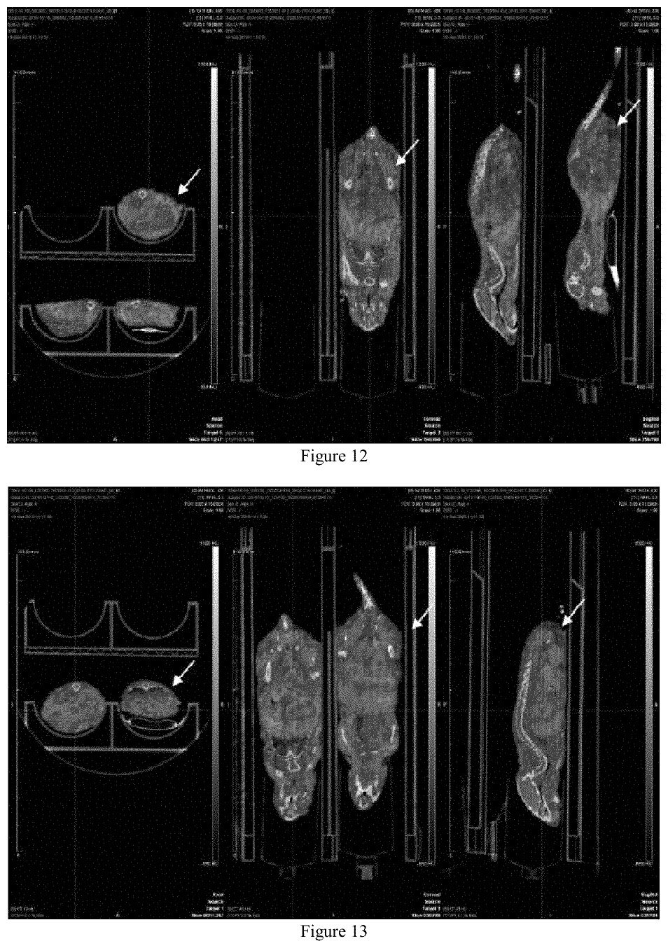

Mice (n=4 per group for MDA-MB-468 and n=3 or 2 for PANC-1) bearing breast or pancreatic xenografts were injected with 89Zr labelled dendrimers via tail-vein. Images were taken at 8 hrs, 24 hrs, 48 hrs, 72 hrs, 144 hrs and 216 hrs post-injection. At 216 hrs post-injection, the organs were removed and signal intensity quantified by ex vivo gamma analysis. Faecal pellets were also measured for activity at this timepoint. FIGS. 10 to 13 show representative images for each dendrimer and xenograft 9 days post-injection of the dendrimer.

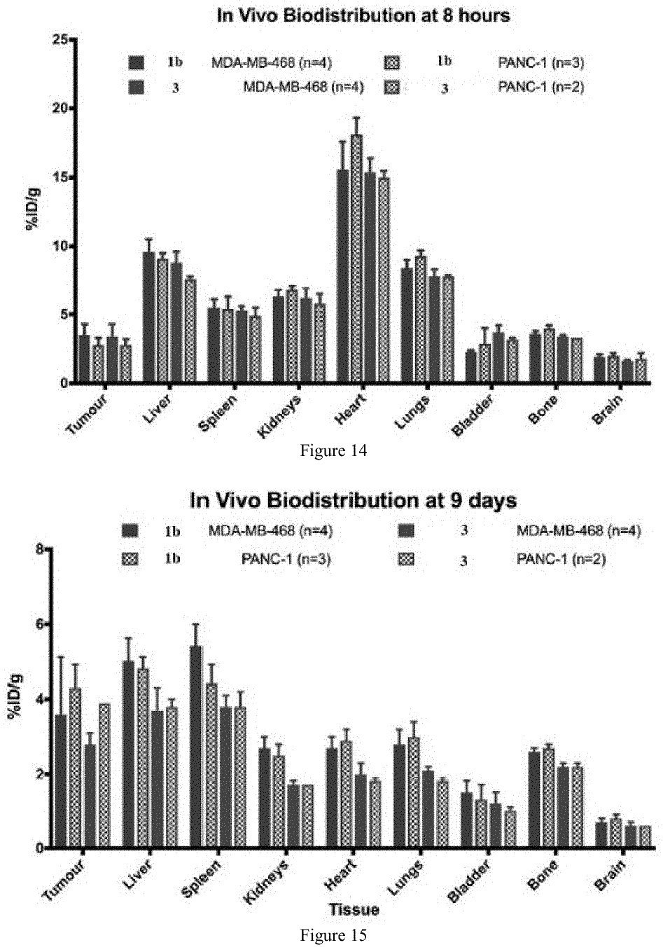

Accumulation plots for the organs as determined in vivo and ex vivo are provided in FIGS. 14, 15, and 19 to highlight trends in biodistribution and clearance.

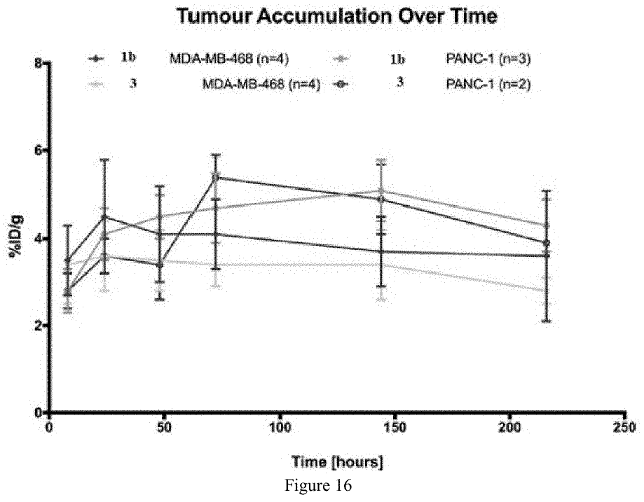

To further evaluate trends in the data, comparisons between the tumour uptake at different timepoints was plotted to show the temporal effect of accumulation as a function of tumour type. This data was extracted from the in vivo images by drawing a region of interest around the tumour mass at the different timepoints, as well as from the ex vivo analyses for comparison. The details are shown in FIG. 16.

### Conclusion

RadioTLC showed that both compound 1b and 3 labelled to high efficiency using standard protocols, and a single purification step was required to achieve >99% purity.

All mice showed good tumour growth and tumour accumulation was shown to reach approximately 4% ID/g for both MDA-MB-468 and PANC-1 tumours using in vivo imaging data. There was no significant difference in the tumour accumulation for the two different dendrimers. Variability did arise between the tumour type (across all four tumour models) and this is likely due to level of vasculature and heterogeneity between tumour types, however this would require further investigation including tissue analysis.

In terms of rate of accumulation, all dendrimers show slow accumulation up to 3-6 days at which time maximum uptake is observed. This is indicative of an EPR mechanism contributing to the accumulation owing to long circulation of the dendrimers. At longer times, the signal starts to decrease and this could be indicative of both processing of the dendrimer by cells in the tumour tissue (either tumour cells or immune cells) and/or slow loss of imaging probe (either through decomplexation or degradation).

No unusual accumulation in clearance organs was observed, with the liver and spleen signal showing expected concentration ranges as typically observed for similar systems. The presence of significant signal in the faeces of all animal cohorts at 6 days suggests that both dendrimers are still being cleared through this route. Likewise, in vivo images show that there is statistically significant signal intensity in the blood at 9 days for all animals, indicating that the dendrimers also have a proportion that is still circulating at this point. The signal intensity measured in the bone sample at 9 days post-injection across all tumour models showed signals that were around or just slightly higher than background, suggesting that there was minimal accumulation in this tissue.

### Example 21

### Tumour Imaging Study With Radionuclide-containing Dendrimers—Glioblastoma

The aim of this study was to assess the level of accumulation of example radionuclide-containing dendrimers in mice bearing spontaneous gliomas. This model provides a route to effectively assess the ability to both cross the blood-brain-barrier (BBB) as well as accumulate in tumour tissue.

### Mouse Model

All breeding and experiments were performed in accordance with the Australian Code of Practice for the Care and Use of Animals for Scientific Purposes and with approval from the Animal Ethics Committee.

Gt(ROSA)26So^{rtm14(CAG-tdTomato)Hze} 20023653 was crossed with Pten^{tm2MAK}; Rb1^{tm2Brn}; Trp53^{tm1Bm}; Tg(GFAP-cre/Esr1*,−lacZ)BSbk^{31,44-47} (alleles) and backcrossed six generations to latter mice to generate Gt(ROSA)26Sor^{tm14(AG-tdTomato)Hze}; Pten^{tm2MAK}; Rb1^{tm2Brn}. Trp53^{tm1Bm}; Tg(GFAP-cre/Esr1*,−lacZ)BSbk (high grade glioma mouse model; HGG). Mice were maintained on a predominantly FVB/NJ background with contributions from 129/SV and C57Bl6. To induce Cre recombinase and thereby tumor formations, 20 mg/ml Tamoxifen (Sigma-Aldrich) dissolved in corn oil (Sigma-Aldrich) was injected intraperitoneally. Up to 200 mg/kg body weight was administered weekly for 3 consecutive weeks after postnatal day (P) 30 (range P30-44). Animal's health and welfare was monitored up to twice daily and animals were euthanized based on morbidity requirement.

### Radiolabelling and TLC Analysis of Dendrimers.

91 μL Zr-89 oxalate in 1 M oxalic acid (Perkin Elmer) was diluted with 78 μL 1 M Na₂CO₃ to neutralize pH. Dendrimer 1b was dissolved in 0.5 M HEPES (pH 7.5). 33 μL neutralized Zr-89 stock (approximately 15 MBq) was added to the dendrimer (146 μg) to give 100-fold excess of the dendrimer to Zr-89 and labelling was allowed to proceed at room temperature for 1 hour. The sample was then buffer exchanged into phosphate-buffered saline using Zeba Spin Desalting Columns (7 kDa MWCO, Thermo Fisher Scientific). 1 μL sample of the solution was taken and spotted on thin layer chromatography paper (Agilent iTLC-SG Glass microfiber chromatography paper impregnated with silica gel) and run with 50 mM diethylenetriaminepentaacetic acid (DTPA) as the eluent. Control experiments were conducted to monitor the elution behaviour of unbound Zr-89 for quality control. Plates were then imaged on a Bruker In Vivo MS FX Pro imaging system using a radioisotopic phosphor screen. ˜100% chelation of the Zr-89 was observed and so the dendrimer was used directly for the imaging experiments.

### PET-MRI Imaging

Anaesthetized mice, with a cannulated tail vein, were placed in a combined MRI/PET system, comprising a 300 mm bore 7T ClinScan, running Siemens VB17, and removable PET insert containing 3 rings of 16 detector blocks with 15×15 LSO crystals (1.6×1.6×10 mm) per block, at the centre of the magnet bore operating under Siemens Inveon Acquisition Workplace (IAW) (Bruker, Germany). A 23 mm ID mouse head MRI RF coil inside the PET ring was used to acquire mouse head images simultaneously with the PET acquisition.

Mice were injected with approximately 5 MBq of Zr-89 labelled dendrimer 1b and imaged 40 hours and 5 days post-injection. At each imaging point a dose of Gadovist® contrast agent was injected to obtain pre- and post-contrast T1, T2, and dynamic image data. The injection dose at each timepoint was comprised of 50 μl Gadovist® diluted with PBS (1×) to give a total volume of 200 μl. This volume was injected via a catheter inserted into the tail vein in a slow bolus injection. Where collected, dynamic PET data acquisition was performed for 60 min. Prior to injection, fast localizer images and a 3D T1 weighted volumetric interpolated breath-hold examination VIBE sequence was acquired. Dynamic MRI images were acquired with a Gradient echo FLASH sequence, with 3 slices acquired each 2 seconds interval. The PET acquisition and dynamic MRI imaging was started simultaneously, a 2-3 min baseline period acquired and then the solution was injected. Following 15 min of dynamic MRI scanning, the T1 weighted VIBE was repeated, structural T2 weighted spin echo images acquired and a 3T T1 weighted VIBE_DIXON sequence acquired to generate a 3D T1 map.

The PET data was reconstructed using dedicated PET reconstructed software developed by the University of Tubingen for the PET insert. PET images with a matrix of 128×128×89 were reconstructed using the ordered-subset expectation maximization (OSEM2D) algorithm. MRI and PET datasets were aligned using IRW software (Siemens) using a transformation matrix generated using a phantom with known features.

### Image Processing

All MRI images were acquired using the ClinScan software mentioned above, and subtraction images were calculated using the built-in function. All images were exported as DICOMS from the ClinScan software and further processed and analysed with Osirix MD for the dynamic uptake, T1- and T2-weighted images as MRI alone (v 9.0.1). PET data and resulting generated PET-MRI fusion maximum intensity projection images were prepared using Siemens Inveon Research Workplace software.

### Data Analysis

Data was aggregated in Microsoft Excel (Mac 2016, v 16,9) and basic mathematic calculations were done were done with in the worksheets. All plots were made with GraphPad Prism 7 and all statistical analyses and area under curve measurements were done using the built-in functions. Calculations for radiotracer uptake are presented as percent injected dose per gram (% ID/g) and were calculated from the in vivo images using Siemens Inveon Research Workplace.

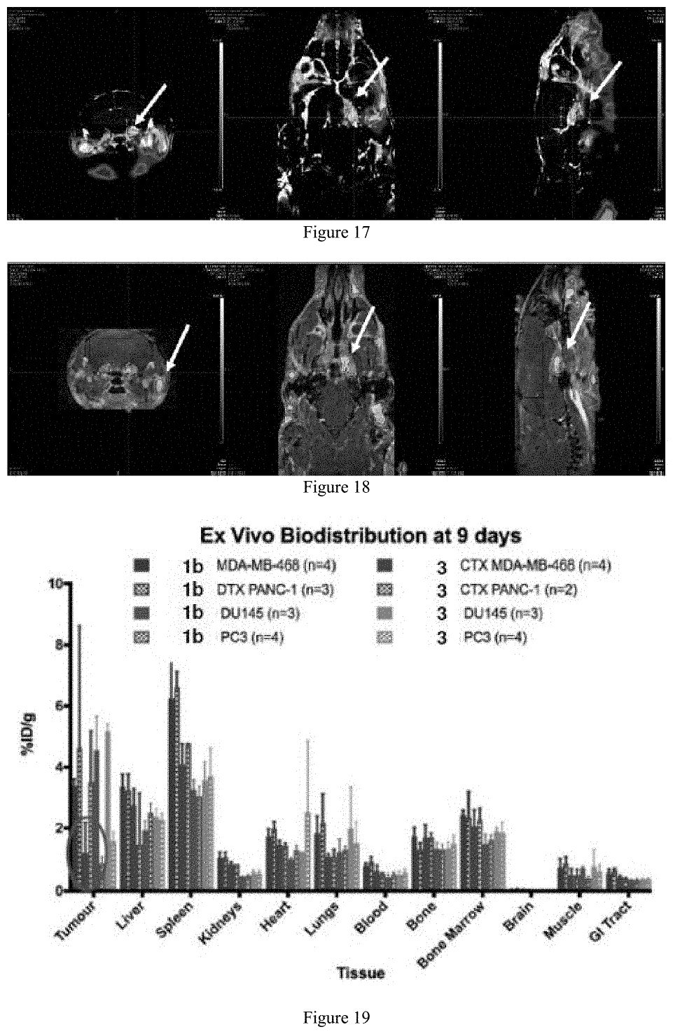

PET-MR images were acquired at 40 hours and 5 days postinjection of SPL 9149, and are shown in FIGS. 17 and 18. The region of the tumour is shown with white arrows. Other signal intensity is from blood flow around skull of mouse in highly vascularised areas.

The relative uptake and accumulation of compound 1b radiolabelled with Zr-89 compared to brain and vascalature (brain accumulation is determined by measuring the signal intensity in a region of the brain distant to the tumour) at different timepoints is shown in the table below:

| Organ | % ID/g at 40 hrs | % ID/g at 5 days |
|---|---|---|
| Brain (minus tumour) | 2.0 | 1.5 |
| Vascalature | 2.3 | 2.0 |
| Tumour | 4.3 | 4.0 |

### Conclusions

The accumulation of this dendrimer in the brain tumour was found to be high. The images indicate that the dendrimer crosses the BBB and accumulates in tumour tissue to a much higher extent than other regions, indicating therapeutic potential for brain tumours.

### Example 22

Therapeutic Study with Radionuclide-Containing Dendrimers

### Animal Model

Healthy male Balb/C nude mice (˜20 g) from 8 weeks old were obtained from the ARC and used for this study. Mice were imported into the animal holding facility and monitored for 1 week prior to the study in order to acclimatise to the environment prior to injection of cells. All animals were provided with free access to food and water before and during the imaging experiments which were approved by the Animal Ethics Committee.

### Dendrimers

The following compounds were used in the study:
Example 4b
Example 5
Jevtana® (cabazitaxel)
Comparative example A: a non-radionuclide-containing dendrimer, which is BHALys[Lys]₃₂[α-DGA-Cabazitaxel]_{32†}[ε-PEG_{˜2100}]_{32‡}.
   Note: 32‡ relates to the theoretical number of ε surface amino groups on the dendrimer available for substitution with PEG_{˜2100}. The actual mean number of PEG_{˜2100} groups attached to the BHALys[Lys]₃₂ was determined experimentally by ¹H NMR.

### Radiolabelling and TLC Analysis of Dendrimers.

All dendrimers were incubated with Lu-177 at a 100-fold excess of polymer in 0.1 M pH 5.5 ammonium acetate buffer for 60 minutes at 37° C. Samples of each solution were taken and mixed 2:1 with 50 mM DTPA. 5 μL of each solution was spotted on TLC paper (Agilent iTLC-SG Glass microfiber chromatography paper impregnated with silica gel) and run with 50:50 water:ethanol (v/v). Plates were then imaged on a Carestream MSFX imaging system using a radioisotopic phosphor screen. Where necessary, unbound copper was removed by purification using 7 K MWCO Zeba Spin Columns (Thermo Scientific) as per manufacturers protocols. Dendrimers exhibited >95% labelling and were used for the subsequent regression study. For each of the analyses discussed, radioisotopic TLCs were obtained by mixing samples with an excess of DTPA (50 mM) to scavenge any unbound Lu-177. In this TLC system, dendrimers will remain at the baseline (R_{f}=0) while DTPA-complexed Lu-177 will move with the solvent front to the top of the paper (R_{f}=1).

### Tumour Initiation and Growth

78 mice were injected with 4×10⁶ DU-145 cells in Matrigel into the right flank to induce subcutaneous tumours. Tumour volume and body mass was monitored twice per week before mice with evident tumour growth (approximately 100 mm² in volume, tumour volume=½ (length×width²)) were randomly assigned to groups and injected with compounds according to the schedule outlined in the table below, at day 0, 7 and 14. Following injections, the mice were monitored three times per week for tumour volume and body mass. Mice were culled if the tumours reached significant size (>1 cm³), or in accordance with ethical requirements. No mice were culled due to due to treatment regimen.

| | | Day 0 | | |
|---|---|---|---|---|
| | | Drug | Day 7 | Day 14 |
| | | equivalent | Drug | Drug |
| | | dose | equivalent | equivalent |
| Group | Compound | (LU-177 dose) | dose | dose |
| 1 | Control (saline) | 0 | 0 | 0 |
| 2 | Jevtana ® | 8 mg/kg | 8 mg/kg | 8 mg/kg |
| 3 | Comparative Example A | 8 mg/kg | 8 mg/kg | 8 mg/kg |
| 4 | 4b (CTX/DOTA) | 8 mg/kg (6 MBq) | 0 | 0 |
| | Comparative Example A | 0 | 8 mg/kg | 8 mg/kg |
| 5 | Jevtana ® | 5 mg/kg | 5 mg/kg | 5 mg/kg |
| 6 | Comparative Example A | 5 mg/kg | 5 mg/kg | 5 mg/kg |
| 7 | 4b (CTX/DOTA) | 5 mg/kg (6 MBq) | 0 | 0 |
| | Comparative Example A | 0 | 5 mg/kg | 5 mg/kg |
| 8 | 5 (DOTA) | (6 MBq) | 0 | 0 |

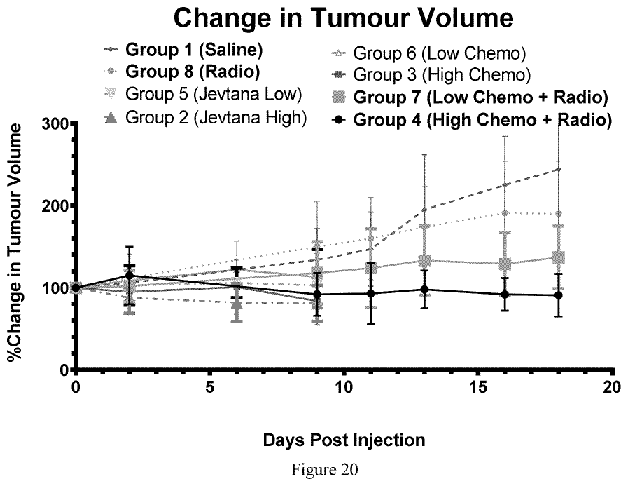

As shown in FIG. 20, radionuclide dendrimer (group 8) and the dendrimer with both cabazitaxel and radionuclide (groups 4 and 7) were all effective in suppressing tumour growth, with the higher dose of compound 4b (group 4) being most effective.

1. A dendrimer comprising:
i) a core unit (C); and
ii) building units (BU),
wherein the core unit is covalently attached to at least two building units;
the dendrimer having from two to six generations of building units; wherein building units of different generations are covalently attached to one another; and
the dendrimer further comprising:
iii) one or more first terminal groups attached to an outermost building unit, wherein each first terminal group comprises a radionuclide-containing moiety; and
iv) one or more second terminal groups attached to an outermost building unit, wherein each second terminal group comprises a pharmacokinetic-modifying moiety;
or a salt thereof.

2. A dendrimer as claimed in claim 1, wherein the first terminal group comprises a complexation group and a radionuclide.

3. A dendrimer as claimed in claim 2, wherein the complexation group is a DOTA, benzyl-DOTA, NOTA, DTPA, sarcophagine or DFO group.

4. A dendrimer as claimed in claim 3, wherein the complexation group is a DOTA, benzyl-DOTA, NOTA, DTPA or DFO group.

5. A dendrimer as claimed in any of claims 1 to 4, wherein the radionuclide in the radionuclide-containing moiety is a lutetium, gadolinium, gallium, zirconium, actinium, bismuth, astatine, technetium or copper radionuclide.

6. A dendrimer as claimed in claim 5, wherein the radionuclide is a gadolinium, zirconium or lutetium radionuclide.

7. A dendrimer as claimed in any of claims 1 to 5, wherein the radionuclide is a copper, zirconium, lutetium, actinium or astatine radionuclide.

8. A dendrimer as claimed in claim 7, wherein the radionuclide is a copper-64, copper-67, zirconium-89, lutetium-177, actinium-225 or an astatine-211 radionuclide.

9. A dendrimer as claimed in any of claims 1 to 8, wherein the radionuclide is an α-emitter.

10. A dendrimer as claimed in any of claims 1 to 9, wherein the radionuclide is a β-emitter.

11. A dendrimer as claimed in any of claims 1 to 10, wherein the pharmacokinetic-modifying moiety is a polyethylene glycol (PEG) group or a polyethyloxazoline (PEOX) group.

12. A dendrimer as claimed in claim 11, wherein the pharmacokinetic-modifying moiety is a PEG group having an average molecular weight of at least 500 Daltons.

13. A dendrimer as claimed in claim 12, wherein the pharmacokinetic-modifying moiety is a PEG group having an average molecular weight in the range of from 500 to 3000 Daltons.

14. A dendrimer as claimed in any of claims 11 to 13, wherein the PEG group is a methoxy-terminated PEG.

15. A dendrimer as claimed in any of claims 1 to 14, wherein the dendrimer comprises a third terminal group attached to an outermost building unit, the third terminal group comprising a residue of a pharmaceutically active agent.

16. A dendrimer as claimed in claim 15, wherein the pharmaceutically active agent is an anti-cancer agent or radiosensitiser.

17. A dendrimer as claimed in claim 16, wherein the anticancer agent is selected from the group consisting of an auristatin, a maytansinoid, a taxane, a topoisomerase inhibitor and a nucleoside analogue.

18. A dendrimer as claimed in claim 17, wherein the anticancer agent is selected from the group consisting of auristatin A, monomethyl auristatin F, cabazitaxel, docetaxel, SN-38 and gemcitabine.

19. A dendrimer as claimed in claim 18, wherein the anticancer agent is selected from the group consisting of cabazitaxel, docetaxel, and SN-38.

20. A dendrimer as claimed in any of claims 15 to 19, wherein the residue of a pharmaceutically active agent is covalently attached to an outermost building unit via a linker.

21. A dendrimer as claimed in claim 20, wherein the residue of a pharmaceutically active agent is covalently attached to an outermost building unit via a cleavable linker.

22. A dendrimer as claimed in claim 21, wherein the linker is

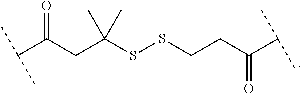

23. A dendrimer as claimed in any of claims 1 to 22, wherein the core unit does not provide an attachment point for a terminal group other than via the building units.

24. A dendrimer as claimed in any of claims 1 to 23, wherein the generations of building units are complete generations.

25. A dendrimer as claimed in any of claims 1 to 24, wherein the core unit is covalently attached to at least two building units via amide linkages, each amide linkage being formed between a nitrogen atom present in the core unit and the carbon atom of an acyl group present in a building unit.

26. A dendrimer as claimed in any of claim 25, wherein the core unit of the dendrimer is formed from a core unit precursor comprising two amino groups.

27. A dendrimer as claimed in claim 26, wherein the core unit is:

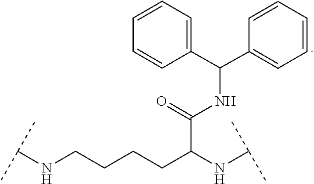

28. A dendrimer as claimed in any of claims 1 to 27, wherein building units of different generations are covalently attached to one another via amide linkages formed between a nitrogen atom present in one building unit and the carbon atom of an acyl group present in another building unit.

29. A dendrimer as claimed in claim 28, wherein the building units are lysine residues or analogues thereof.

30. A dendrimer as claimed in claim 29, wherein the building units are each:

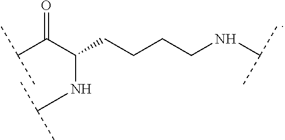

31. A dendrimer as claimed in any of claims 28 to 30, wherein the first terminal group is attached to the nitrogen atom of an outermost building unit, and the second terminal group is attached to the nitrogen atom of an outermost building unit.

32. A dendrimer as claimed in claim 31, wherein from 1 to 3 of the nitrogen atoms present in the outermost building units are attached to a first terminal group.

33. A dendrimer as claimed in claim 31 or 32, wherein at least 40% of the nitrogen atoms present in the outermost building units are attached to a second terminal group.

34. A dendrimer as claimed in any of claims 28 to 33, wherein the dendrimer comprises a third terminal group attached to the nitrogen atom of an outermost building unit, the third terminal group comprising a residue of a pharmaceutically active agent.

35. A dendrimer as claimed in claim 34, wherein the pharmaceutically active agent comprises a hydroxyl group, wherein the residue of a pharmaceutically active agent is covalently attached via the oxygen atom of the hydroxyl group through a cleavable linker to an outermost building unit, and wherein the cleavable linker is a diacyl linker group of formula 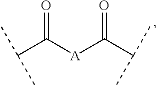
wherein A is a C₂-C₁₀ alkylene group which is optionally interrupted by O, S, S—S, NH, or N(Me), or in which A is a heterocycle selected from the group consisting of tetrahydrofuran, tetrahydrothiophene, pyrrolidine and N-methylpyrrolidine.

36. A dendrimer as claimed in claim 35, wherein the diacyl linker is

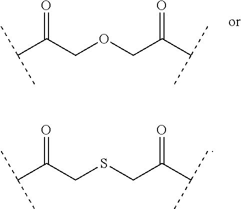

39. A dendrimer as claimed in any of claims 34 to 36, wherein at least one third of the nitrogen atoms present in the outermost building units are attached to a third terminal group.

40. A dendrimer as claimed in any of claims 28 to 39, wherein the dendrimer comprises outermost building units which contain —NH₂ groups and/or which contain a nitrogen atom which is capped with an acetyl group.

41. A dendrimer as claimed in any of claims 28 to 40, wherein at least 80% of the nitrogen atoms present in the outermost generation of building units are substituted.

42. A dendrimer as claimed in any of claims 1 to 41, wherein the dendrimer comprises surface units comprising an outer building unit and a second terminal group of the formula. 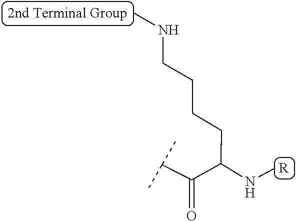
wherein R represents a first terminal group or a third terminal group.

43. A dendrimer as claimed in any of claims 1 to 42, wherein the dendrimer is any of the Example dendrimers.

44. A composition comprising a plurality of dendrimers or salts thereof, wherein at least some of the dendrimers are as defined in any of claims 1 to 43, and wherein the mean number of first terminal groups per dendrimer in the composition is in the range of from 0.2 to 8, and the mean number of second terminal groups per dendrimer in the composition is in the range of from 10 to 32.

45. A composition as claimed in claim 44, wherein the mean number of third terminal group per dendrimer in the composition is in the range of from 10 to 31.

46. A composition as claimed in claim 44 or 45, wherein the composition is a pharmaceutical composition comprising a pharmaceutically acceptable excipient.

47. A method of determining whether a subject has a cancer, comprising:
administering to a subject a dendrimer as claimed in any of claims 1 to 43 or a pharmaceutical composition as claimed in claim 46;
carrying out imaging on the subject's body or a part thereof; and
determining whether the subject has a cancer based on the imaging results.

48. A method of imaging a cancer in a subject, comprising:
administering to a subject having a cancer a dendrimer as claimed in any of claims 1 to 43 or a pharmaceutical composition as claimed in claim 46;
carrying out imaging on the subject's body or a part thereof.

49. A method of determining the progression of a cancer in a subject, comprising:
administering to a subject having a cancer a first amount of a dendrimer as claimed in any of claims 1 to 43 or a pharmaceutical composition as claimed in claim 46;
carrying out a first imaging step on the subject's body or a part thereof;
subsequently administering to the subject a second amount of a dendrimer as claimed in any of claims 1 to 43 or a pharmaceutical composition as claimed in claim 46;
carrying out a second imaging step on the subject's body or a part thereof; and
determining whether the cancer has progressed based on the first and second imaging results.

50. A method of determining an appropriate therapy for a subject having a cancer, comprising:
administering to the subject a dendrimer as claimed in any of claims 1 to 43 or a pharmaceutical composition as claimed in claim 46;
carrying out imaging on the subject's body or a part thereof; and
if the imaging results indicate susceptibility of the cancer to treatment with a therapy, administering the therapy to the subject.

51. A method of determining the effectiveness of a cancer therapy administered to a subject having a cancer, comprising:
administering to the subject a first amount of a dendrimer as claimed in any of claims 1 to 43 or a pharmaceutical composition as claimed in claim 46;
carrying out a first imaging step on the subject's body or a part thereof;
administering to the subject a cancer therapy;
subsequently administering to the subject a second amount of a dendrimer as claimed in any of claims 1 to 43 or a pharmaceutical composition as claimed in claim 46;
carrying out a second imaging step on the subject's body or a part thereof; and
determining the effectiveness of the cancer therapy based on the first and second imaging results.

52. A method as claimed in claim 50 or 51, wherein the therapy is a dendrimer as claimed in any of claims 1 to 43 or a pharmaceutical composition as claimed in claim 46.

53. A method of treating cancer comprising administering to a subject in need thereof a therapeutically effective amount of a dendrimer as claimed in any of claims 1 to 43 or a pharmaceutical composition as claimed in claim 46.

54. A dendrimer as claimed in any of claims 1 to 43, or a pharmaceutical composition as claimed in claim 46, for use in the diagnosis of cancer in a subject, for use in determining an appropriate therapy for a subject having a cancer, for use in determining the effectiveness of a cancer therapy administered to a subject, or for use in determining the progression of a cancer in a subject.

55. A dendrimer as claimed in any of claims 1 to 43, or a pharmaceutical composition as claimed in claim 46, for use in the treatment of cancer.

56. Use of a dendrimer as claimed in any of claims 1 to 43, or of a pharmaceutical composition as claimed in claim 46, in the manufacture of a medicament for the diagnosis of cancer, for determining an appropriate therapy for a subject having a cancer, for determining the effectiveness of a cancer therapy administered to a subject, or for determining the progression of a cancer in a subject.

57. Use of a dendrimer as claimed in any of claims 1 to 43, or of a pharmaceutical composition as claimed in claim 46, in the manufacture of a medicament for the treatment of cancer.

58. A method, use, or dendrimer or composition for use as claimed in any of claims 47 to 57, wherein the cancer is prostate cancer, pancreatic cancer, gastrointestinal cancer, stomach cancer, lung cancer, uterine cancer, breast cancer, brain cancer or ovarian cancer.

59. A method, use, or dendrimer or composition for use as claimed in claim 58, wherein the cancer is prostate cancer, pancreatic cancer, breast cancer or brain cancer.

60. A method, use, or dendrimer or composition for use as claimed in any of claims 58 to 59, wherein the cancer is a brain cancer of a glioblastoma, meningioma, pituitary, nerve sheath, astrocytoma, oligodendroglioma, ependymoma, medulloblastoma, or craniopharyngioma.

61. A method, use, or dendrimer or composition for use as claimed in any of claims 58 to 60, wherein the dendrimer is administered in combination with a further anti-cancer drug.

62. An intermediate for producing a radionuclide-containing dendrimer which comprises:
i) a core unit (C); and
ii) building units (BU);
wherein the core unit is covalently attached to at least two building units;
the dendrimer having from two to six generations of building units; wherein building units of different generations are covalently attached to one another; and
the dendrimer further comprising:
iii) one or more first terminal groups attached to an outermost building unit, wherein each first terminal group comprises a complexation group for complexing a radionuclide; and
iv) one or more second terminal groups attached to an outermost building unit, wherein each second terminal group comprises a pharmacokinetic-modifying moiety.

63. A kit for producing a dendrimer as defined in any of claims 1 to 43, comprising:
a) an intermediate as defined in claim 62; and
b) a radionuclide.

64. A process for producing a dendrimer as claimed in any of claims 1 to 43, comprising:
contacting an intermediate as defined in claim 62 with a radionuclide, thereby producing the dendrimer.
